SEQUENCE LISTING (66 sequences)

SEQ ID NO: 1 - protein, 124 aa, synthetic construct
EVQLVESGGGLVQPGGSLRLSCAASGFTFSSYAMSWVRQAPGKGLEWVSSVSGSGGNTYYADSVKGRFTISRDNSKNTLYLQMSGLRAEDTAVYYCAKMEYNNKGYAYFFMEVWGKGTTVTVSS

SEQ ID NO: 2 - protein, 8 aa, synthetic construct
GFTFSSYA

SEQ ID NO: 3 - protein, 8 aa, synthetic construct
VSGSGGNT

SEQ ID NO: 4 - protein, 17 aa, synthetic construct
AKMEYNNKGYAYFFMEV

SEQ ID NO: 5 - protein, 5 aa, synthetic construct
SYAMS

SEQ ID NO: 6 - protein, 17 aa, synthetic construct
SVSGSGGNTYYADSVKG

SEQ ID NO: 7 - protein, 15 aa, synthetic construct
MEYNNKGYAYFFMEV

SEQ ID NO: 8 - protein, 124 aa, synthetic construct
EVQLVESGGGLVQPGGSLRLSCAASGFTFSSYAMSWVRQAPGKGLEWVSSVSGSGGNTYYADSVKGRFTISRDNSKNTLYLQMNSLRAEDTAVYYCAKMEYNNKGYAYFFMEVWGKGTTVTVSS

SEQ ID NO: 9 - protein, 124 aa, synthetic construct
EVQLVESGGGLVQPGGSLRLSCAASGFTFSNYAMSWVRQAPGQGLEWASSISDSGGNTYSADSMKGRFTISRDNSKNTLYLQMNSLRAEDTAVYYCARFIRSAKGYSYYYMDVWGKGTMVTVSS

SEQ ID NO: 10 - protein, 8 aa, synthetic construct
GFTFSNYA

SEQ ID NO: 11 - protein, 8 aa, synthetic construct
ISDSGGNT

SEQ ID NO: 12 - protein, 17 aa, synthetic construct
ARFIRSAKGYSYYYMDV

SEQ ID NO: 13 - protein, 5 aa, synthetic construct
NYAMS

SEQ ID NO: 14 - protein, 17 aa, synthetic construct
SISDSGGNTYSADSMKG

SEQ ID NO: 15 - protein, 15 aa, synthetic construct
FIRSAKGYSYYYMDV

SEQ ID NO: 16 - protein, 118 aa, synthetic construct
EVQLVQSGAEVKKPGSSVKVSCKASGGTFRSFGISWVRQAPGQGLEWMGGFIPVLGTANYAQKFQGRVTIIADKSTNTAYMELSSLRSEDTAVYYCARRGNWNPFDPWGQGTLVTVSS

SEQ ID NO: 17 - protein, 8 aa, synthetic construct
GGTFRSFG

SEQ ID NO: 18 - protein, 8 aa, synthetic construct
FIPVLGTA

SEQ ID NO: 19 - protein, 11 aa, synthetic construct
ARRGNWNPFDP

SEQ ID NO: 20 - protein, 5 aa, synthetic construct
SFGIS

SEQ ID NO: 21 - protein, 17 aa, synthetic construct
GFIPVLGTANYAQKFQG

SEQ ID NO: 22 - protein, 9 aa, synthetic construct
RGNWNPFDP

SEQ ID NO: 23 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 24 - protein, 6 aa, synthetic construct
QSISSY

SEQ ID NO: 25 - skipped

SEQ ID NO: 26 - protein, 9 aa, synthetic construct
QQSYSTPPT

SEQ ID NO: 27 - protein, 11 aa, synthetic construct
RASQSISSYLN

SEQ ID NO: 28 - protein, 7 aa, synthetic construct
AASSLQS

SEQ ID NO: 29 - protein, 9 aa, synthetic construct
QQSYSTPPT

SEQ ID NO: 30 - protein, 107 aa, synthetic construct [sequence omitted]

SEQ ID NO: 31 - protein, 95 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 32 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 33 - protein, 10 aa, synthetic construct
QQSYSTPPIT

SEQ ID NO: 34 - protein, 95 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWP

SEQ ID NO: 35 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIK

SEQ ID NO: 36 - protein, 6 aa, synthetic construct
QSVSSN

SEQ ID NO: 37 - skipped

SEQ ID NO: 38 - protein, 9 aa, synthetic construct
QQYNNWPWT

SEQ ID NO: 39 - protein, 96 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 40 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 41 - protein, 7 aa, synthetic construct
QSVSSSY

SEQ ID NO: 42 - skipped

SEQ ID NO: 43 - protein, 9 aa, synthetic construct
QQYGSSPWT

SEQ ID NO: 44 - protein, 96 aa, synthetic construct
SYVLTQPPSVSVAPGETARITCGGDNIGRKSVYWYQQKSGQAPVLVIYYDSDRPSGIPERFSGSNSGNTATLTISRVEAGDEADYYCQVWDGSSDH

SEQ ID NO: 45 - protein, 108 aa, synthetic construct
SYVLTQPPSVSVAPGETARITCGGDNIGRKSVYWYQQKSGQAPVLVIYYDSDRPSGIPERFSGSNSGNTATLTISRVEAGDEADYYCQVWDGSSDHWVFGGGTKLTVL

SEQ ID NO: 46 - protein, 6 aa, synthetic construct
NIGRKS

SEQ ID NO: 47 - skipped

SEQ ID NO: 48 - protein, 11 aa, synthetic construct
QVWDGSSDHWV

SEQ ID NO: 49 - protein, 15 aa, synthetic construct
EPKSCDKTHTCPPCP

SEQ ID NO: 50 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 51 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 52 - protein, 110 aa, synthetic construct
APELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 53 - protein, 107 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 107 aa, synthetic construct
GQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 107 aa, synthetic construct
GQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 417 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 57 - protein, 411 aa, synthetic construct
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRTRRMMRTKMRMRRMRRTRRKMRRKMSPARPRTSCREACLQGWTEA

SEQ ID NO: 58 - protein, 411 aa, synthetic construct
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNDPSIYAYDNFGVLGLDLWQVKSGTIFDNFLITNDEAYAEEFGNETWGVTKAAEKQMKDKQDEEQRLKEEEEDKKRKEEEEAEDNCRRMMRTKMRMRRMRRTRRKMRRKMSPARPRTSCREACLQGWTEA

SEQ ID NO: 59 - protein, 454 aa, synthetic construct
EVQLVESGGGVVQPGRSLRLSCAASGFTFSNYGMHWVRQAPGKGLEWVAVISYDGSTKYSADSLKGRFTISRDNSKNTLYLQMNSLRADDTAVYYCAKEGWSFDSSGYRSWFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 456 aa, synthetic construct
EVQLVETGAEVKKPGASVKVSCKASDYIFTKYDINWVRQAPGQGLEWMGWMSANTGNTGYAQKFQGRVTMTRDTSINTAYMELSSLTSGDTAVYFCARSSLFKTETAPYYHFALDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 197 aa, synthetic construct
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLE

SEQ ID NO: 63 - protein, 453 aa, synthetic construct
EVQLVESGGGLVQPGGSLRLSCAASGFTFSSYAMSWVRQAPGKGLEWVSSVSGSGGNTYYADSVKGRFTISRDNSKNTLYLQMSGLRAEDTAVYYCAKMEYNNKGYAYFFMEVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPG

SEQ ID NO: 64 - protein, 453 aa, synthetic construct
EVQLVESGGGLVQPGGSLRLSCAASGFTFSNYAMSWVRQAPGQGLEWASSISDSGGNTYSADSMKGRFTISRDNSKNTLYLQMNSLRAEDTAVYYCARFIRSAKGYSYYYMDVWGKGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPG

SEQ ID NO: 65 - protein, 453 aa, synthetic construct
EVQLVESGGGLVQPGGSLRLSCAASGFTFSSYAMSWVRQAPGKGLEWVSSVSGSGGNTYYADSVKGRFTISRDNSKNTLYLQMNSLRAEDTAVYYCAKMEYNNKGYAYFFMEVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPG

SEQ ID NO: 66 - protein, 447 aa, synthetic construct
EVQLVQSGAEVKKPGSSVKVSCKASGGTFRSFGISWVRQAPGQGLEWMGGFIPVLGTANYAQKFQGRVTIIADKSTNTAYMELSSLRSEDTAVYYCARRGNWNPFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2026/0209343 A1
(43) Pub. Date: Jul. 23, 2026

(54) MULTISPECIFIC BINDING MOIETIES COMPRISING CALR AND CD3 BINDING DOMAINS

(71) Applicants: Merus N.V., Utrecht (NL); Incyte Corporation, Wilmington, DE (US)

(72) Inventors: David Andre Baptiste MAUSSANG-DETAILLE, Utrecht (NL); Sanne Laura Jacqueline DEMANDT, Utrecht (NL); Patrick MAYES, Wilmington, DE (US); Liang-Chuan WANG, Wilmington, DE (US); Horacio G. NASTRI, Wilmington, DE (US); Stephen RUDNICK, Wilmington, DE (US)

(73) Assignees: Merus N.V., Utrecht (NL); Incyte Corporation, Wilmington, DE (US)

(21) Appl. No.: 19/262,599

(22) Filed: Jul. 8, 2025

(30) Foreign Application Priority Data
Jul. 9, 2024 (NL) 2038169
Apr. 11, 2025 (NL) 2040168

(51) Int. Cl. C07K 16/28; A61K 39/00; A61P 35/00; C12N 15/63

(52) U.S. Cl. CPC C07K 16/28; A61P 35/00; C07K 16/2809; C12N 15/63; A61K 2039/505; C07K 2317/31; C07K 2317/35; C07K 2317/522; C07K 2317/524; C07K 2317/526; C07K 2317/53; C07K 2317/565; C07K 2317/77

(57) ABSTRACT

The present disclosure relates to binding domains that bind Calreticulin (CALR) and binding domain that binds to CD3, as well as binding moieties comprising such binding domains. The present disclosure further relates to the use of such binding domains or binding moieties in the treatment of cancer. The present disclosure also relates to nucleic acid encoding such binding domains or binding moieties, and a vector and cell comprising such nucleic acid.

### INCORPORATION OF SEQUENCE LISTING

A sequence listing in electronic (XML file) format is filed with this application and incorporated herein by reference. The name of the file is AttachD_SequenceListing; the file was created on Jul. 3, 2025; and the size of the file is 61,559 bytes.

### TECHNICAL FIELD

The present disclosure relates to the field of antibodies. In particular it relates to the field of therapeutic antibodies for the treatment of disease, in particular for the treatment of cancer. More particularly it relates to bispecific binding moieties comprising a binding domain that binds to Calreticulin (CALR) and a binding domain that binds to CD3.

### BACKGROUND

Two major subgroups of myeloproliferative neoplasms (MPN) are essential thrombocythemia (ET) and primary myelofibrosis (MF), which lead to overproduction of platelets and gradual replacement of normal bone marrow tissue with a fibrous scar-like material, respectively. The exact cause of MPN remains unknown, however, mutations in the Calreticulin (CALR) gene have been described in patients with ET and MF (Klampfl et al. Somatic Mutations of Calreticulin in Myeloproliferative Neoplasms. N Engl J Med. 2013; 369:2379-2390 and Nagalia et al. Somatic CALR Mutations in Myeloproliferative Neoplasms with Nonmutated JAK2. N Engl J med. 2013; 369:2391-2405).

CALR is an endoplasmic reticulum (ER) chaperone protein that plays a role in calcium homeostasis and glycoprotein quality control. It contains three domains: an N-terminal lectin domain (N), a proline-rich domain (P), and an acidic carboxyl domain (C) which contains KDEL, an ER-retention motif (Smith et al. Multiple zones in the sequence of Calreticulin (CRP55, Calregulin, HACBP), a major calcium binding ER/SR protein. EMBO J. 1989; 8:3581-3586). CALR is also known under a number of different aliases such as Calregulin; CC1qR; CRT; SSA; RO; Sicca Syndrome Antigen A; Endoplasmic Reticulum Resident Protein 60; FLJ26680; CALR1; CRP55; ERp60; HACBP; Grp60. External Ids for CALR are HGNC:1455; NCBI Gene:811; Ensembl: ENSG00000179218; OMIM®: 109091; UniProtKB/Swiss-Prot: P27797.

CALR mutations described so far include insertions or deletions causing a +1 frameshift within exon 9 which could generate a novel C-terminal sequence and the loss of CALR's ER retention signal (Prins et al. Mutant Calreticulin in the Myeloproliferative Neoplasms. Hemasphere. 2020 Jan. 15; 4(1):e333). The two most frequently occurring CALR mutations are type I and II, a 52 base pair deletion (del52) and a 5 base pair insertion (ins5), respectively. These CALR mutants (CALRmut) loose the ER retention signal KDEL and acquire the ability to bind to and activate the thrombopoietin receptor (TPO-R) on the cell surface, leading to activation of the JAK2 pathway and dysregulated megakaryopoiesis as reported for ET and MF. In contrast, wild-type CALR (CALRwt) is retained in the ER.

CALRmut is expressed at the cell surface through a stable interaction with the extracellular domain of myeloproliferative leukemia protein (MPL), and as such can be targeted specifically in MPN. The use of T cell engagers, such as CD3 T cell engagers, may redirect cytotoxic T cells to specifically target CALRmut expressing cells. Both CALR mutants del52 and ins5 contain a cleavage site within the novel mutant end tail (Kihara, Y.; Araki, M.; Imai, M.; Mori, Y.; Horino, M.; Ogata, S.; Yoshikawa, S.; Taguchi, T.; Masubuchi, N.; Mabuchi, Y.; et al. Therapeutic Potential of an Antibody Targeting the Cleaved Form of Mutant Calreticulin in Myeloproliferative Neoplasms. Blood 2020, 136(Suppl 1), 9-10. https://doi.org/10.1182/blood-2020-141159). CD3 T cell engagers specifically targeting mutant CALR have been generated that bind mutant CALR within the domain not affected by cleavage of the protein.

The term “CD3” (cluster of differentiation 3) refers to a protein complex, which is composed of a CD3γ chain (SwissProt P09693), a CD3δ chain (SwissProt P04234), CD3ε chains (SwissProt P07766), and a CD3 zeta chain homodimer (SwissProt P20963). CD3ε is known under various aliases some of which are: “CD3e Molecule, Epsilon (CD3-TCR Complex)”; “CD3e Antigen, Epsilon Polypeptide (TiT3 Complex)”; T-Cell Surface Antigen T3/Leu-4 Epsilon Chain; T3E; T-Cell Antigen Receptor Complex, Epsilon Subunit Of T3; CD3e Antigen; CD3-Epsilon 3; IMD18; TCRE. Ids for CD3E Gene are HGNC: 1674; Entrez Gene: 916; Ensembl: ENSG00000198851; OMIM: 186830 and UniProtKB: P07766. These chains associate with the T-cell receptor (TCR) and the ζ-chain to form a TCR complex that can upon mitogenic signaling generate an activation signal in T lymphocytes. CD3 is expressed on T cells and NK T cells.

Therapies for ET and MF are mainly focusing on controlling the symptoms and extending the lifespan of patients by reducing their thrombohemorrhagic risk. Therefore, there remains a need for novel therapeutic interventions that target CALRmut/MPL-expressing cells.

### SUMMARY

One of the objects of the present disclosure is to provide a new pharmaceutical agent for the treatment of human disease, in particular for the treatment of cancer. This object is met by the provision of Calreticulin (CALR) binding domains. This object is further met by the provision of bispecific binding moieties that bind CALR and CD3.

In certain embodiments, the present disclosure provides a polypeptide comprising an amino acid sequence as described herein, or a variant thereof, as well as methods for producing such variant.

In certain embodiments, the present disclosure provides a CALR binding domain comprising a polypeptide as described herein.

In certain embodiments, the present disclosure provides a binding moiety comprising a CALR binding domain as described herein.

In certain embodiments, the present disclosure provides a binding moiety comprising a CALR binding domain which binds the N-domain of CALR.

In certain embodiments, the present disclosure provides a bispecific binding moiety comprising a CALR binding domain and a CD3 binding domain.

In certain embodiments, the present disclosure provides a bispecific binding moiety comprising a CALR binding domain and a CD3 binding domain, wherein the bispecific binding moiety has one or more properties selected from:
high T cell-mediated tumor cell killing;
no or low internalization by human T cells;
low potency to induce cytokine release syndrome;
low clearance in a mouse pharmacokinetics (PK) study.

In certain embodiments, the present disclosure provides a bispecific binding moiety comprising a CALR binding domain and a CD3 binding domain, wherein the CALR binding domain comprises a heavy chain variable region comprising CDR1, CDR2, and CDR3 sequences as described herein.

In certain embodiments, the present disclosure provides a bispecific binding moiety comprising a CALR binding domain and a CD3 binding domain, wherein the CD3 binding domain comprises a heavy chain variable region comprising CDR1, CDR2, and CDR3 sequences as described herein.

In certain embodiments, the present disclosure provides a bispecific binding moiety that binds CALR and CD3, wherein the bispecific binding moiety competes with a bispecific binding moiety as described herein for binding to CALR and/or CD3.

In certain embodiments, the present disclosure provides a pharmaceutical composition comprising an effective amount of the bispecific binding moiety as described herein.

In certain embodiments, the present disclosure provides a bispecific binding moiety as described herein, or a pharmaceutical composition as described herein, for use in therapy.

In certain embodiments, the present disclosure provides a bispecific binding moiety as described herein, or a pharmaceutical composition as described herein, for use in the treatment of cancer.

In certain embodiments, the present disclosure provides a bispecific binding moiety as described herein, or a pharmaceutical composition as described herein, for use in the treatment of myeloproliferative neoplasms (MPN).

In certain embodiments, the present disclosure provides a bispecific binding moiety as described herein, or a pharmaceutical composition as described herein, for use in the treatment of essential thrombocythemia (ET).

In certain embodiments, the present disclosure provides a bispecific binding moiety as described herein, or a pharmaceutical composition as described herein, for use in the treatment of myelofibrosis (MF).

In certain embodiments, the present disclosure provides a method for treating a disease, comprising administering an effective amount of a bispecific binding moiety as described herein, or a pharmaceutical composition as described herein, to an individual in need thereof.

In certain embodiments, the present disclosure provides a method for treating cancer, comprising administering an effective amount of a bispecific binding moiety as described herein, or a pharmaceutical composition as described herein, to an individual in need thereof.

In certain embodiments, the present disclosure provides a method for treating myeloproliferative neoplasms (MPN), comprising administering an effective amount of a bispecific binding moiety as described herein, or a pharmaceutical composition as described herein, to an individual in need thereof.

In certain embodiments, the present disclosure provides a method for treating essential thrombocythemia (ET), comprising administering an effective amount of a bispecific binding moiety as described herein, or a pharmaceutical composition as described herein, to an individual in need thereof.

In certain embodiments, the present disclosure provides a method for treating myelofibrosis (MF), comprising administering an effective amount of a bispecific binding moiety as described herein, or a pharmaceutical composition as described herein, to an individual in need thereof.

In certain embodiments, the present disclosure provides a nucleic acid sequence encoding the heavy chain variable region of a CALR binding domain as described herein and/or a nucleic acid sequence encoding the heavy chain variable region of a CD3 binding domain as described herein.

In certain embodiments, the present disclosure provides a vector comprising a nucleic acid sequence as described herein.

In certain embodiments, the present disclosure provides a cell comprising a nucleic acid sequence encoding the heavy chain variable region of a CALR binding domain as described herein and/or a nucleic acid sequence encoding the heavy chain variable region of a CD3 binding domain as described herein.

In certain embodiments, the present disclosure provides a cell producing a CALR binding domain or a bispecific binding moiety as described herein.

### DETAILED DESCRIPTION

One of the objects of the present disclosure is to provide a new pharmaceutical agent for the diagnosis and treatment of disease, in particular in humans and in particular for the diagnosis and treatment of cancer. This object is met by the provision of Calreticulin (CALR) binding domains and the provision of bispecific binding moieties that bind CALR and CD3.

In certain embodiments, the present disclosure provides a polypeptide comprising the amino acid sequence as set forth in SEQ ID NO: 1, 8, or 9, or a variant thereof. In certain embodiments, the polypeptide is an immunoglobulin heavy chain, or part thereof, that, when combined with a suitable light chain, or part thereof, binds to CALR. For example, the part of an immunoglobulin heavy chain can be a heavy chain variable region with a CH1 region, or a heavy chain variable region. The part of a light chain can, for example, be a light chain variable region.

In certain embodiments, the present disclosure provides a polypeptide comprising the amino acid sequence as set forth in SEQ ID NO: 63, 64 or 65. In certain embodiments, the polypeptide is an immunoglobulin heavy chain, or part thereof, that, when combined with a suitable light chain, or part thereof, binds to CALR.

In certain embodiments, the polypeptide when combined with a suitable light chain, or part thereof, binds to human CALR, in particular to wildtype human CALR and mutant human CALR. The amino acid sequence of human wildtype CALR is provided as SEQ ID NO: 56. Mutant human CALR includes human CALRmut del52 and human CALRmut ins5. The amino acid sequence of human CALRmut del52 is provided as SEQ ID NO: 57 and the amino acid sequence of human CALRmut ins5 is provided as SEQ ID NO: 58.

In certain embodiments, the polypeptide when combined with a suitable light chain, or part thereof, binds to isolated human CALR, in particular to isolated wildtype human CALR.

In certain embodiments, the polypeptide when combined with a suitable light chain, or part thereof, does not bind to cell surface expressed wildtype human CALR. In certain embodiments, the polypeptide when combined with a suitable light chain, or part thereof, binds to cell surface expressed mutant human CALR. In certain embodiments, the polypeptide when combined with a suitable light chain, or part thereof, binds to cell surface expressed human CALR with a mutated C-terminus, for example cell surface expressed human CALRmut del52 and cell surface expressed human CALRmut ins5.

In certain embodiments, the polypeptide when combined with a suitable light chain, or part thereof, binds to the N-domain, also referred to as the N-terminal domain, of human CALR. The amino acid sequence of the N-domain of human CALR is provided as SEQ ID NO: 62.

In certain embodiments, the present disclosure provides variants of the polypeptides as described herein. Such variants can be produced by making a modification in the amino acid sequence of any one of SEQ ID Nos: 1, 8, or 9. A modification can be one or more modifications, such as for instance one or more amino acid substitutions, insertions, deletions, or a combination thereof. In certain embodiments, the modification is limited to at most one, two, three, four, five, six, seven, eight, nine, or ten modifications. In certain embodiments, the modification is limited to at most one, two, three, four or five amino acid substitutions. In certain embodiments, the modification is limited to one, two, or three amino acid substitutions.

In certain embodiments, the present disclosure provides a CALR binding domain that comprises a polypeptide comprising the amino acid sequence as set forth in SEQ ID NO: 1, 8, or 9.

In certain embodiments, the present disclosure provides a CALR binding domain that comprises a polypeptide comprising the amino acid sequence as set forth in SEQ ID NO: 63, 64 or 65.

In general, as described herein, antigen binding can be expressed in terms of specificity and affinity. The specificity determines which antigen or epitope thereof is specifically bound by the binding domain or binding moiety. The affinity is a measure for the strength of binding to a particular antigen or epitope.

In certain embodiments, the CALR binding domain further comprises a polypeptide comprising the amino acid sequence as set forth in SEQ ID NO: 23.

In certain embodiments, the present disclosure provides a CALR binding domain comprising a heavy chain variable region comprising the heavy chain CDR1 (HCDR1), heavy chain CDR2 (HCDR2), and heavy chain CDR3 (HCDR3) of a heavy chain variable region having the amino acid sequence selected from SEQ ID NOs: 1, 8, and 9. In certain embodiments, each of the HCDRs may comprise at most three, two, or one amino acid variations. In certain embodiments, only one of the HCDR1 or HCDR2 may comprise at most three, two, or one amino acid variations. In certain embodiments, HCDR3 does not comprise any amino acid variations. In certain embodiments, the amino acid variation is a conservative amino acid substitution.

In general, as described herein, typically, a conservative amino acid substitution involves a variation of an amino acid with a homologous amino acid residue, which is a residue that shares similar characteristics or properties. Homologous amino acids are known in the art, as are routine methods for making amino acid substitutions in antibody binding domains without significantly impacting binding or function of the antibody, see for instance handbooks like Lehninger (Nelson, David L., and Michael M. Cox. 2017. Lehninger Principles of Biochemistry. 7th ed. New York, NY: W.H. Freeman) or Stryer (Berg, J., Tymoczko, J., Stryer, L. and Stryer. L., 2007. Biochemistry. New York: W.H. Freeman), incorporated herein in its entirety. In determining whether an amino acid can be replaced with a conserved amino acid, an assessment may typically be made of factors such as, but not limited to, (a) the structure of the polypeptide backbone in the area of the substitution, for example, a sheet or helical conformation, (b) the charge or hydrophobicity of the molecule at the target site, and/or (c) the bulk of the side chain(s). If a residue can be substituted with a residue which has common characteristics, such as a similar side chain or similar charge or hydrophobicity, then such a residue is preferred as a substitute. For example, the following groups can be determined: (1) non-polar: Ala (A), Gly (G), Val (V), Leu (L), Ile (I), Pro (P), Phe (F), Trp (W), Met (M); (2) uncharged polar: Ser (S), Thr (T), Cys (C), Tyr (Y), Asn (N), Gln (Q); (3) acidic: Asp (D), Glu (E); and (4) basic: Lys (K), Arg (R), His (H). Alternatively, the amino acids may be grouped as follows: (1) aromatic: Phe (F), Trp (W), Tyr (Y); (2) apolar: Leu (L), Val (V), Ile (I), Ala (A), Met (M); (3) aliphatic: Ala (A), Val (V), Leu (L), Ile (I); (4) acidic: Asp (D), Glu (E); (5) basic: His (H), Lys (K), Arg (R); and (6) polar: Gln (Q), Asn (N), Ser (S), Thr (T), Tyr (Y). Alternatively, amino acid residues may be divided into groups based on common side-chain properties: (1) hydrophobic: Met (M), Ala (A), Val (V), Leu (L), Ile (I); (2) neutral hydrophilic: Cys (C), Ser (S), Thr (T), Asn (N), Gln (Q); (3) acidic: Asp (D), Glu (E); (4) basic: His (H), Lys (K), Arg R); (5) residues that influence chain orientation: Gly (G), Pro (P); and (6) aromatic: Trp (W), Tyr (Y), Phe (F).

The substitution of an amino acid residue with another present in the same group would be preferred. Accordingly, conservative amino acid substitution can involve exchanging a member of one of these classes for another member of that same class. Typically, the variation results in no, or substantially no, loss in binding specificity of the binding domain to its intended target.

Additional types of amino acid variations include variations resulting from somatic hypermutation or affinity maturation. Binding variants encompassed by the present disclosure include somatically hypermutated or affinity matured heavy chain variable regions, which are heavy chain variable regions derived from the same VH gene segments as the heavy chain variable regions described by sequence herein, the variants having amino acid variations, including non-conservative and/or conservative amino acid substitutions in one, two, or all three HCDRs. Routine methods for affinity maturing antibody binding domains are widely known in the art, see for instance Tabasinezhad M. et al. (Trends in therapeutic antibody affinity maturation: From in-vitro towards next-generation sequencing approaches. Immunol Lett. 2019 August; 212:106-113).

In certain embodiments, the present disclosure provides a CALR binding domain that comprises a heavy chain variable region comprising:
a) heavy chain CDR1 (HCDR1), heavy chain CDR2 (HCDR2), and heavy chain CDR3 (HCDR3), having the amino acid sequence as set forth in SEQ ID NO: 2, SEQ ID NO: 3, and SEQ ID NO: 4, respectively, or heavy chain CDR1 (HCDR1), heavy chain CDR2 (HCDR2), and heavy chain CDR3 (HCDR3), having the amino acid sequence as set forth in SEQ ID NO: 5, SEQ ID NO: 6, and SEQ ID NO: 7, respectively, or
b) heavy chain CDR1 (HCDR1), heavy chain CDR2 (HCDR2), and heavy chain CDR3 (HCDR3), having the amino acid sequence as set forth in SEQ ID NO: 10, SEQ ID NO: 11, and SEQ ID NO: 12, respectively, or heavy chain CDR1 (HCDR1), heavy chain CDR2 (HCDR2), and heavy chain CDR3 (HCDR3), having the amino acid sequence as set forth in SEQ ID NO: 13, SEQ ID NO: 14, and SEQ ID NO: 15, respectively. In certain embodiments, each of the HCDRs may comprise at most three, two, or one amino acid variations. In certain embodiments, only one of the HCDR1 or HCDR2 may comprise at most three, two, or one amino acid variations. In certain embodiments, HCDR3 does not comprise any amino acid variations. In certain embodiments, the amino acid variation is a conservative amino acid substitution.

In certain embodiments, the CALR binding domain comprises a heavy chain variable region having the amino acid sequence as set forth in SEQ ID NO: 1, 8, or 9, or having at least 80%, at least 85%, at least 90%, or at least 95% sequence identity thereto.

In certain embodiments, the CALR binding domain comprises a heavy chain having the amino acid sequence as set forth in SEQ ID NO: 63, 64 or 65, or having at least 80%, at least 85%, at least 90%, or at least 95% sequence identity thereto.

In certain embodiments, the CALR binding domain comprises a heavy chain having the amino acid sequence as set forth in SEQ ID NO: 63, 64 or 65, and a light chain having the amino acid sequence as set forth in SEQ ID NO: 60.

“Percent (%) identity” as referring to nucleic acid or amino acid sequences herein is defined as the percentage of residues in a candidate sequence that are identical with the residues in a selected sequence, after aligning the sequences for optimal comparison purposes. In order to optimize the alignment between the two sequences, gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/bases or amino acids. The sequence identity is the percentage of identical matches between the two sequences over the reported aligned region.

A comparison of sequences and determination of percentage of sequence identity between two sequences can be accomplished using a mathematical algorithm. The skilled person will be aware of the fact that several different computer programs are available to align two sequences and determine the identity between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley). The percent sequence identity between two amino acid sequences or nucleic acid sequences may be determined using the Needleman and Wunsch algorithm for the alignment of two sequences. (Needleman, S. B. and Wunsch, C. D. (1970) J. Mol. Biol. 48, 443-453). The Needleman-Wunsch algorithm has been implemented in the computer program Geneious Prime (Dotmatics). For the purpose of this disclosure, the Geneious Prime program and the Geneious alignment, Global alignment Needleman-Wunsch with Blosum62 cost matrix, are used to determine the percent identity of amino acid sequences. The parameters used are a gap-open penalty of 12 and a gap extension penalty of 3. For DNA sequences, the Geneious Prime program and the Geneious alignment, Global alignment Needleman-Wunsch with cost matrix Identity (1.0/0.0) are used. The parameters used are a gap-open penalty of 12 and a gap extension penalty of 3.

After alignment by the program Geneious Prime as described above, the percentage of sequence identity between a query sequence and a sequence of this disclosure is calculated as follows: Number of corresponding positions in the alignment showing an identical amino acid or identical nucleotide in both sequences divided by the total length of the alignment after subtraction of the total number of gaps in the alignment.

In certain embodiments, a CALR binding domain of the present disclosure also comprises CALR binding domain variants, which, in addition to the variations in the HCDRs referred to above, comprise one or more variations in the framework regions. A variation can be any type of amino acid variation described herein, such as for instance a conservative amino acid substitution or non-conservative amino acid substitution resulting from somatic hypermutation or affinity maturation. In certain embodiments, a CALR binding domain variant of the present disclosure comprises no variations in the CDR regions but comprises one or more variations in the framework regions. Such variants have at least 80%, or at least 85%, or at least 90%, or at least 95% sequence identity to the sequences disclosed herein, and are expected to retain CALR binding specificity. Thus, in certain embodiments, a CALR binding domain of the present disclosure comprises:
a heavy chain variable region having at least 8%, or at least 85%, or at least 90%, or at least 95% sequence identity to the amino acid sequence as set forth in SEQ ID NO: 1, which heavy chain variable region comprises a HCDR1 amino acid sequence as set forth in SEQ ID NO: 2; a HCDR2 amino acid sequence as set forth in SEQ ID NO: 3; and a HCDR3 amino acid sequence as set forth in SEQ ID NO: 4, or a HCDR1 amino acid sequence as set forth in SEQ ID NO: 5; a HCDR2 amino acid sequence as set forth in SEQ ID NO: 6; and a HCDR3 amino acid sequence as set forth in SEQ ID NO: 7;
a heavy chain variable region having at least 80%, preferably 85%, more preferably 90%, or most preferably 95% sequence identity to the amino acid sequence as set forth in SEQ ID NO: 8, which heavy chain variable region comprises a HCDR1 amino acid sequence as set forth in SEQ ID NO: 2; a HCDR2 amino acid sequence as set forth in SEQ ID NO: 3; and a HCDR3 amino acid sequence as set forth in SEQ ID NO: 4, or a HCDR1 amino acid sequence as set forth in SEQ ID NO: 5; a HCDR2 amino acid sequence as set forth in SEQ ID NO: 6; and a HCDR3 amino acid sequence as set forth in SEQ ID NO: 7; or
a heavy chain variable region having at least 80%, preferably 85%, more preferably 90%, or most preferably 95% sequence identity to the amino acid sequence as set forth in SEQ ID NO: 9, which heavy chain variable region comprises a HCDR1 amino acid sequence as set forth in SEQ ID NO: 10; a HCDR2 amino acid sequence as set forth in SEQ ID NO: 11; and a HCDR3 amino acid sequence as set forth in SEQ ID NO: 12, or a HCDR1 amino acid sequence as set forth in SEQ ID NO: 13; a HCDR2 amino acid sequence as set forth in SEQ ID NO: 14; and a HCDR3 amino acid sequence as set forth in SEQ ID NO: 15.

In certain embodiments, the CALR binding domain comprises a light chain or light chain variable region as described herein.

In certain embodiments, the CALR binding domain comprises one or more constant regions as described herein.

In certain embodiments, the present disclosure provides a method for producing a variant of a CALR binding domain of the present disclosure, as well as a CALR binding domain obtainable thereby, wherein the method comprises:
making a modification in the amino acid sequence as set forth in SEQ ID NOS: 1, 8, or 9, and/or in the amino acid sequence as set forth in SEQ ID NO: 23;
testing the modified CALR binding domain for binding to human wildtype and/or mutant CALR, and
selecting the modified CALR binding domain if it binds to human wildtype and/or mutant CALR.

A modification can be one or more modifications, such as for instance one or more amino acid substitutions, insertions, deletions, or a combination thereof. In certain embodiments, the modification is limited to at most one, two, three, four, five, six, seven, eight, nine, or ten modifications. In certain embodiments, the modification is limited to at most one, two, three, four or five amino acid substitutions. In certain embodiments, the modification is limited to one, two, or three amino acid substitutions.

In certain embodiments, the present disclosure provides a binding moiety comprising a CALR binding domain as described herein.

In general, as described herein, a “binding moiety” refers to a proteinaceous molecule and includes for instance all antibody formats available in the art, such as for example a full length IgG antibody, immunoconjugates, diabodies, BiTEs, Fab fragments, scFv, tandem scFv, single domain antibody (like VHH and VH), minibodies, scFab, scFv-zipper, nanobodies, DART molecules, TandAb, Fab-scFv, F(ab)′2, F(ab)′2-scFv2, and intrabodies as well as any other formats known to a person of ordinary skill in the art.

In certain embodiments, a binding moiety is a monospecific binding moiety, in particular a bivalent monospecific antibody. A monospecific antibody according to the present disclosure is an antibody, in any antibody format, that comprises one or more binding domains with specificity for a single target or epitope. In certain embodiments, a monospecific binding moiety of the present disclosure may further comprise an Fc region or a part thereof. In certain embodiments, a monospecific binding moiety of the present disclosure is an IgG1 antibody.

In general, as described herein, an “Fc region” typically comprises a hinge, CH2, and CH3 region. Suitable hinge, CH2, and CH3 regions include, but are not limited to, those as described herein. The Fc region mediates effector functions of an antibody, such as complement-dependent cytotoxicity (CDC), antibody-dependent cellular cytotoxicity (ADCC) and antibody-dependent cell phagocytosis (ADCP). Depending on the therapeutic antibody or Fc fusion protein application, it may be desired to either reduce or increase the effector function.

In certain embodiments, a binding moiety of the present disclosure has Fc effector function. In certain embodiments, a binding moiety of the present disclosure has enhanced Fc effector function. In certain embodiments, a binding moiety of the present disclosure exhibits antibody-dependent cell-mediated cytotoxicity (ADCC). A binding moiety, such as an antibody, can be engineered to enhance the ADCC activity (for review, see Kubota T et al. Cancer Sci. 2009; 100(9):1566-72). For instance, ADCC activity of an antibody can be improved when the antibody itself has a low ADCC activity, by slightly modifying the constant region of the antibody (Junttila T T. et al. Cancer Res. 2010; 70(11):4481-9). Changes are sometimes also made to improve storage or production or to remove C-terminal lysines (Kubota T et al. Cancer Sci. 2009; 100(9):1566-72). Another way to improve ADCC activity of an antibody is by enzymatically interfering with the glycosylation pathway resulting in a reduced fucose (von Horsten H H. et al. Glycobiology. 2010; 20(12):1607-18). Alternatively, or additionally, multiple other strategies can be used to achieve ADCC enhancement, for instance including glycoengineering (Kyowa Hakko/Biowa, GlycArt (Roche) and Eureka Therapeutics) and mutagenesis, all of which seek to improve Fc binding to low-affinity activating FcγRIIIa, and/or to reduce binding to the low affinity inhibitory FcγRIIb. In certain embodiments, a binding moiety of the present disclosure exhibits enhanced antibody-dependent cell-mediated cytotoxicity (ADCC). In certain embodiments, a binding moiety of the present disclosure is afucosylated.

In certain embodiments, the Fc region of the bispecific binding moiety has reduced immune cell effector function, in particular reduced ADCC and/or ADCP activity. In certain embodiments, a bispecific binding moiety of the present disclosure has reduced Fc-receptor interaction or reduced C1q binding. In certain embodiments, a bispecific binding moiety of the present disclosure exhibits reduced ADCC and/or ADCP. A bispecific binding moiety, such as an antibody, can be engineered to reduce the ADCC and/or ADCP activity (Liu R, et. al. Fc-Engineering for Modulated Effector Functions-Improving Antibodies for Cancer Treatment. Antibodies (Basel). 2020 Nov. 17; 9(4):64). For instance, ADCC and/or ADCP activity of an antibody can be reduced by modifying the CH2 and/or lower hinge region of an IgG antibody, such that the interaction of the antibody to a Fc-gamma receptor is reduced. In certain embodiments, the binding moiety is a bispecific binding moiety.

In certain embodiments, a bispecific binding moiety is a bispecific antibody. In certain embodiments, a bispecific binding moiety is a bivalent bispecific antibody. A bispecific antibody according to the present disclosure is an antibody that comprises at least two binding domains which have specificity for two different targets or epitopes.

In certain embodiments, a bispecific antibody of the present disclosure may comprise an Fc region or a part thereof. In certain embodiments, a bispecific binding moiety of the present disclosure is an IgG1 antibody.

In certain embodiments, the present disclosure provides a bispecific binding moiety that binds to human CALR and human CD3.

In certain embodiments, the present disclosure provides a bispecific binding moiety comprising a CALR binding domain and a CD3 binding domain, wherein the bispecific binding moiety has one or more properties selected from:
high T cell-mediated tumor cell killing, as defined by having a potency with an AUC of between 2000-4000 in a T cell-mediated cytotoxicity assay, or as defined by having a 2-10 fold higher AUC than untreated cells in a T-cell mediated cytotoxicity assay;
no or low internalization by human T cells, as defined by having an internalization rate with an AUC of between 0-25 in an internalization assay;
low risk to induce cytokine release syndrome, as defined by having a 2-10 fold lower potency than antibody vibecotamab in a PBMC cytokine release assay;
low clearance in a cynomolgus monkey pharmacokinetics (PK) study, as defined by having a clearance rate of between 0.07 to 1.14 ml/h/kg.

For the purposes of the present disclosure, determining if a bispecific binding moiety has a high T cell-mediated tumor cell killing is done by using an assay described herein. Therefore, in certain embodiments, the potency of a bispecific binding moiety of the present disclosure is measured in an assay using TF-1 cells edited to express human CALRmut del52 or ins5. In certain embodiments, the potency of a bispecific binding moiety of the present disclosure is measured in an assay as described in Example 3.

In brief, the assay as described in Example 3 is performed by using labeled TF-1 target cells expressing no human CALRwt as a control and cells expressing human CALRmut del52 or ins5. Bispecific binding moieties are incubated with the target cells at concentrations ranging from 10⁻²-10³ g/ml for 72 hours. Potency is established by measuring the AUC.

In certain embodiments, high T cell-mediated tumor cell killing is as defined by a potency with an AUC of between 2000-4000 in the T cell-mediated cytotoxicity assay using TF1 target cells expressing human CALRmut del52. In certain embodiments, high T cell-mediated tumor cell killing is as defined by a potency with an AUC of between 2000-5000 in the T cell-mediated cytotoxicity assay using TF1 target cells expressing human CALRmut ins5. In certain embodiments, high T cell-mediated tumor cell killing is as defined by a potency with an AUC of between 3000-4000 in the T cell-mediated cytotoxicity assay using TF1 target cells expressing human CALRmut ins5. In certain embodiments, high T cell-mediated tumor cell killing is as defined by having a 2-10 fold higher AUC than untreated cells in a T-cell mediated cytotoxicity assay.

For the purposes of the present disclosure, determining if a bispecific binding moiety has no or low internalization by human T cells is done by using an assay described herein. Therefore, in certain embodiments, the internalization of a bispecific binding moiety of the present disclosure is measured in an assay as described in Example 5.

In brief, the assay as described in Example 5 is performed by using bispecific antibodies labeled with Incucyte human Fab Fluor pH antibody dye. The dye used in these experiments fluoresce red in the acidic environment of the lysosome, indicating internalization of the labeled antibody. The antibodies are incubated at a final concentration of 5 μg/ml together with T cells. Internalization is measured by generating scans every hour for 24 hours capturing brightfield and fluorescence with each scan. Antibody internalization is calculated as the percentage of red cells among the cell population, and the AUC is calculated.

In certain embodiments, no or low internalization by human T cells is as defined by having an internalization rate with an AUC of between 0-22, in the internalization assay. In certain embodiments, no or low internalization by human T cells is as defined by having an internalization rate with an AUC of between 0-22, in the internalization assay. In certain embodiments, no or low internalization by human T cells is as defined by having an internalization rate with an AUC of between 15-22, in the internalization assay.

For the purposes of the present disclosure, determining if a bispecific binding moiety has a low risk to induce cytokine release syndrome is done by using an assay described herein. Therefore, in certain embodiments, the ability of a bispecific binding moiety of the present disclosure to induce cytokine release syndrome is measured in an assay as described in Example 8.

In brief, the assay as described in Example 8 is performed using cells that express human CALRmut del52 or human CALRmut ins5. Test and control antibodies are added to the target cells at different concentrations. PBMCs from healthy donor are added at 5:1 E:T ratio. Cells are incubated for 72 hours at 37° C. After 72 hours, supernatant is collected and cytokine analysis is carried out on the supernatants.

In certain embodiments, a low risk to induce cytokine release syndrome is defined by having a 2-10 fold lower potency, expressed in AUC, than antibody vibecotamab in inducing IL-6, IL-10, and/or TNF-α release, in the PBMC cytokine release assay. In certain embodiments, a low risk to induce cytokine release syndrome is defined by having a 2-5 fold lower potency, expressed in AUC, than antibody vibecotamab in inducing IL-6, IL-10, and/or TNF-α release, in the PBMC cytokine release assay.

In certain embodiments, a low risk to induce cytokine release syndrome is defined by having a 2-fold, 3-fold, or 4-fold lower potency, expressed in AUC, than antibody vibecotamab in inducing IL-6 release, in the PBMC cytokine release assay.

In certain embodiments, a low risk to induce cytokine release syndrome is defined by having a 2-fold, 3-fold, 4-fold, 5-fold, 6-fold, or 7-fold lower potency, expressed in AUC, than antibody vibecotamab in inducing IL-10 release, in the PBMC cytokine release assay.

In certain embodiments, a low risk to induce cytokine release syndrome is defined by having a 2-fold lower potency, expressed in AUC, than antibody vibecotamab in inducing TNF-α release, in the PBMC cytokine release assay.

In certain embodiments, a low risk to induce cytokine release syndrome is defined by having a 2-10 fold lower potency, expressed in AUC, than antibody vibecotamab in inducing IL-10 release, in the PBMC cytokine release assay.

In certain embodiments, a low risk to induce cytokine release syndrome is defined by having a 2-3 fold lower potency, expressed in AUC, than antibody vibecotamab in inducing IL-6 release, a 2-5 fold lower potency, expressed in AUC, than antibody vibecotamab in inducing IL-10, and a 2-3 fold lower potency, expressed in AUC, than antibody vibecotamab in inducing TNF-α release, in the PBMC cytokine release assay.

In certain embodiments, a low risk to induce cytokine release syndrome is defined by having a 2-4 fold lower potency, expressed in AUC, than antibody vibecotamab in inducing IL-6 release, a 2-5 fold lower potency, expressed in AUC, than antibody vibecotamab in inducing IL-10, and a 2-3 fold lower potency, expressed in AUC, than antibody vibecotamab in inducing TNF-α release, in the PBMC cytokine release assay.

The PK attributes of approved therapeutic antibodies in human have a wide range with reported systemic half-lives from 79 to 648 hours and clearance rates of 0.141 to 1.017 ml/h/kg. The PH of conventional antibodies in non-human primates also varies widely with serum half-lives ranging from 29-299 hours and clearance rates of 0.07 to 1.14 ml/h/kg (Walker et al.; 2019; PLOS ONE; 14:5).

For the purposes of the present disclosure, determining if a bispecific binding moiety has a low clearance in a cynomolgus monkey PK study is done by using an experiment described herein. Therefore, in certain embodiments, the clearance of a bispecific binding moiety of the present disclosure is measured in an assay as described in Example 6. In brief, the assay as described in Example 6 is performed by administering antibodies to cynomolgus monkeys via 30 minutes intravenous infusion at 0.6 and 3 mg/kg. Blood samples are collected at 0.5, 1, 4, 12, 24, 48, 120, 168, 216, 336, 504 and 672 hours post-dose. The serum concentrations of antibodies are determined and used to calculate PK parameters. Clearance is calculated by Winnolin's non-compartmental analysis using equation Dose/AUCinf.

In certain embodiments, the bispecific binding moiety binds to wildtype CALR, the amino acid sequence of which is set forth in SEQ ID NO: 56, and to mutant CALR. Mutant CALR includes at least the mutants of which the amino acid sequence is set forth in SEQ ID NO: 57 and 58.

In certain embodiments, the bispecific binding moiety binds to isolated human CALR, in particular to isolated wildtype human CALR.

In certain embodiments, the bispecific binding moiety does not bind to cell surface expressed wildtype human CALR. In certain embodiments, the bispecific binding moiety binds to cell surface expressed mutant human CALR. In certain embodiments, the bispecific binding moiety binds to cell surface expressed human CALR with a mutated C-terminus, for example cell surface expressed human CALRmut del52 and cell surface expressed human CALRmut ins5.

In certain embodiments, the bispecific binding moiety binds to the N-domain, also referred to as the N-terminal domain, of human CALR. The amino acid sequence of N-domain of human CALR is provided as SEQ ID NO: 62.

In certain embodiments, the CALR binding domain of a bispecific binding moiety of the present disclosure comprises a heavy chain variable region comprising the heavy chain CDR1 (HCDR1), heavy chain CDR2 (HCDR2), and heavy chain CDR3 (HCDR3) of a heavy chain variable region having the amino acid sequence selected from SEQ ID NO: 1, 8, and 9. In certain embodiments each of the HCDRs may comprise at most three, two, or one amino acid variations. In certain embodiments, only one or two HCDRs may comprise at most three, two, or one non-conservative amino acid variations. In certain embodiments, HCDR3 does not comprise any amino acid variations. In certain embodiments, the amino acid variation is a conservative amino acid substitution.

In certain embodiments, the CALR binding domain of a bispecific binding moiety of the present disclosure comprises a heavy chain variable region comprising:
a) heavy chain CDR1 (HCDR1), heavy chain CDR2 (HCDR2), and heavy chain CDR3 (HCDR3), having the amino acid sequence as set forth in SEQ ID NO: 2, SEQ ID NO: 3, and SEQ ID NO: 4, respectively, or heavy chain CDR1 (HCDR1), heavy chain CDR2 (HCDR2), and heavy chain CDR3 (HCDR3), having the amino acid sequence as set forth in SEQ ID NO: 5, SEQ ID NO: 6, and SEQ ID NO: 7, respectively, or
b) heavy chain CDR1 (HCDR1), heavy chain CDR2 (HCDR2), and heavy chain CDR3 (HCDR3), having the amino acid sequence as set forth in SEQ ID NO: 10, SEQ ID NO: 11, and SEQ ID NO: 12, respectively, or heavy chain CDR1 (HCDR1), heavy chain CDR2 (HCDR2), and heavy chain CDR3 (HCDR3), having the amino acid sequence as set forth in SEQ ID NO: 13, SEQ ID NO: 14, and SEQ ID NO: 15, respectively. In certain embodiments each of the HCDRs may comprise at most three, two, or one amino acid variations. In certain embodiments, only one or two HCDRs may comprise at most three, two, or one non-conservative amino acid variations. In certain embodiments, HCDR3 does not comprise any amino acid variations. In certain embodiments, the amino acid variation is a conservative amino acid substitution.

The heavy chain variable regions of the CALR binding domain of a bispecific binding moiety of the present disclosure may comprise a limited number, such as for instance one, two, three, four, five, six, seven, eight, nine, or ten, non-conservative amino acid substitutions, or an unlimited number of conservative amino acid substitutions.

In certain embodiments, a CALR binding domain of a bispecific binding moiety of the present disclosure comprises a heavy chain variable region having an amino acid sequence as set forth in SEQ ID NO: 1, 8, or 9, or a variant thereof. In certain embodiments, the CALR binding domain of a bispecific binding moiety of the present disclosure comprises a heavy chain variable region having the amino acid sequence as set forth in SEQ ID NO: 1, 8, or 9, or having at least 80%, or at least 85%, or at least 90%, or at least 95% sequence identity thereto.

In certain embodiments, a CALR binding domain of a bispecific binding moiety of the present disclosure comprises a heavy chain having an amino acid sequence as set forth in SEQ ID NO: 63, 64 or 65.

In certain embodiments, the CALR binding domain of a bispecific binding moiety of the present disclosure comprises a light chain variable region as described herein.

In certain embodiments, the CD3 binding domain of a bispecific binding moiety of the present disclosure comprises a heavy chain variable region comprising the heavy chain CDR1 (HCDR1), heavy chain CDR2 (HCDR2), and heavy chain CDR3 (HCDR3) of a heavy chain variable region having the amino acid sequence as set forth in SEQ ID NO: 16. In certain embodiments each of the HCDRs may comprise at most three, two, or one amino acid variations. In certain embodiments, only one or two HCDRs may comprise at most three, two, or one non-conservative amino acid variations. In certain embodiments, HCDR3 does not comprise any amino acid variations. In certain embodiments, the amino acid variation is a conservative amino acid substitution.

In certain embodiments, the CD3 binding domain of a bispecific binding moiety of the present disclosure comprises a heavy chain variable region comprising heavy chain CDR1 (HCDR1), heavy chain CDR2 (HCDR2), and heavy chain CDR3 (HCDR3), having an amino acid sequence as set forth in SEQ ID NO: 17, SEQ ID NO: 18, and SEQ ID NO: 19, respectively, or heavy chain CDR1 (HCDR1), heavy chain CDR2 (HCDR2), and heavy chain CDR3 (HCDR3), having an amino acid sequence as set forth in SEQ ID NO: 20, SEQ ID NO: 21, and SEQ ID NO: 22, respectively. In certain embodiments each of the HCDRs may comprise at most three, two, or one amino acid variations. In certain embodiments, only one or two HCDRs may comprise at most three, two, or one non-conservative amino acid variations. In certain embodiments, HCDR3 does not comprise any amino acid variations. In certain embodiments, the amino acid variation is a conservative amino acid substitution.

In certain embodiments, the CD3 binding domain of a bispecific binding moiety comprises a heavy chain variable region having an amino acid sequence as set forth in SEQ ID NO: 16, or having at least 80%, or at least 85%, or at least 90%, or at least 95% sequence identity thereto.

In certain embodiments, a CD3 binding domain of a bispecific binding moiety of the present disclosure comprises a heavy chain having an amino acid sequence as set forth in SEQ ID NO: 66.

In certain embodiments, a CD3 binding domain of the present disclosure also comprises CD3 binding domain variants, which, in addition to the variations in the HCDRs referred to above, comprise one or more variations in the framework regions. A variation can be any type of amino acid variation described herein, such as for instance a conservative amino acid substitution or non-conservative amino acid substitution resulting from somatic hypermutation or affinity maturation. In certain embodiments, a CD3 binding domain variant of the present disclosure comprises no variations in the CDR regions but comprises one or more variations in the framework regions. Such variants have at least 80%, or at least 85%, or at least 90%, or at least 95% sequence identity to the sequences disclosed herein, and are expected to retain CD3 binding specificity. Thus, in certain embodiments, a CD3 binding domain comprises:
a heavy chain variable region having at least 80%, or at least 85%, or at least 90%, or at least 95% sequence identity to the amino acid sequence as set forth in SEQ ID NO: 16, which heavy chain variable region comprises a HCDR1 amino acid sequence as set forth in SEQ ID NO: 17; a HCDR2 amino acid sequence as set forth in SEQ ID NO: 18; and a HCDR3 amino acid sequence as set forth in SEQ ID NO: 19, or a HCDR1 amino acid sequence as set forth in SEQ ID NO: 20; a HCDR2 amino acid sequence as set forth in SEQ ID NO: 21; and a HCDR3 amino acid sequence as set forth in SEQ ID NO: 22.

In certain embodiments, the CD3 binding domain of a bispecific binding moiety of the present disclosure comprises a light chain variable region as described herein.

In certain embodiments, the present disclosure provides a bispecific binding moiety comprising a CALR binding domain and a CD3 binding domain, wherein the CALR binding domain comprises a heavy chain variable region as described herein.

In certain embodiments, the present disclosure provides a bispecific binding moiety comprising a CALR binding domain and a CD3 binding domain, wherein the CD3 binding domain comprises a heavy chain variable region as described herein.

In one embodiment, the present disclosure provides a bispecific binding moiety comprising:
a CALR binding domain comprising a heavy chain CDR1 (HCDR1), heavy chain CDR2 (HCDR2), and heavy chain CDR3 (HCDR3), having an amino acid sequence as set forth in SEQ ID NO: 2, SEQ ID NO: 3, and SEQ ID NO: 4, respectively, or a heavy chain CDR1 (HCDR1), heavy chain CDR2 (HCDR2), and heavy chain CDR3 (HCDR3), having an amino acid sequence as set forth in SEQ ID NO: 5, SEQ ID NO: 6, and SEQ ID NO: 7, respectively; and
a CD3 binding domain comprising a heavy chain CDR1 (HCDR1), heavy chain CDR2 (HCDR2), and heavy chain CDR3 (HCDR3), having an amino acid sequence as set forth in SEQ ID NO: 17, SEQ ID NO: 18, and SEQ ID NO: 19, respectively, or a heavy chain CDR1 (HCDR1), heavy chain CDR2 (HCDR2), and heavy chain CDR3 (HCDR3), having an amino acid sequence as set forth in SEQ ID NO: 20, SEQ ID NO: 21, and SEQ ID NO: 22, respectively.

In one embodiment, the present disclosure provides a bispecific binding moiety comprising:
a CALR binding domain comprising a heavy chain CDR1 (HCDR1), heavy chain CDR2 (HCDR2), and heavy chain CDR3 (HCDR3), having an amino acid sequence as set forth in SEQ ID NO: 10, SEQ ID NO: 11, and SEQ ID NO: 12, respectively, or a heavy chain CDR1 (HCDR1), heavy chain CDR2 (HCDR2), and heavy chain CDR3 (HCDR3), having an amino acid sequence as set forth in SEQ ID NO: 13, SEQ ID NO: 14, and SEQ ID NO: 15, respectively; and
a CD3 binding domain comprising a heavy chain CDR1 (HCDR1), heavy chain CDR2 (HCDR2), and heavy chain CDR3 (HCDR3), having an amino acid sequence as set forth in SEQ ID NO: 17, SEQ ID NO: 18, and SEQ ID NO: 19, respectively, or a heavy chain CDR1 (HCDR1), heavy chain CDR2 (HCDR2), and heavy chain CDR3 (HCDR3), having an amino acid sequence as set forth in SEQ ID NO: 20, SEQ ID NO: 21, and SEQ ID NO: 22, respectively.

In one embodiment, the present disclosure provides a bispecific binding moiety comprising:
a CALR binding domain comprising a heavy chain having an amino acid sequence as set forth in SEQ ID NO: 63; and
a CD3 binding domain comprising a heavy chain having an amino acid sequence as set forth in SEQ ID NO: 66.

In one embodiment, the present disclosure provides a bispecific binding moiety comprising:
a CALR binding domain comprising a heavy chain having an amino acid sequence as set forth in SEQ ID NO: 64; and
a CD3 binding domain comprising a heavy chain having an amino acid sequence as set forth in SEQ ID NO: 66.

In one embodiment, the present disclosure provides a bispecific binding moiety comprising:
a CALR binding domain comprising a heavy chain having an amino acid sequence as set forth in SEQ ID NO: 65; and
a CD3 binding domain comprising a heavy chain having an amino acid sequence as set forth in SEQ ID NO: 66.

In one embodiment, the present disclosure provides a bispecific binding moiety comprising:
a CALR binding domain comprising a heavy chain CDR1 (HCDR1), heavy chain CDR2 (HCDR2), and heavy chain CDR3 (HCDR3), having an amino acid sequence as set forth in SEQ ID NO: 2, SEQ ID NO: 3, and SEQ ID NO: 4, respectively, or a heavy chain CDR1 (HCDR1), heavy chain CDR2 (HCDR2), and heavy chain CDR3 (HCDR3), having an amino acid sequence as set forth in SEQ ID NO: 5, SEQ ID NO: 6, and SEQ ID NO: 7, respectively; and
a CD3 binding domain comprising a heavy chain CDR1 (HCDR1), heavy chain CDR2 (HCDR2), and heavy chain CDR3 (HCDR3), having an amino acid sequence as set forth in SEQ ID NO: 17, SEQ ID NO: 18, and SEQ ID NO: 19, respectively, or a heavy chain CDR1 (HCDR1), heavy chain CDR2 (HCDR2), and heavy chain CDR3 (HCDR3), having an amino acid sequence as set forth in SEQ ID NO: 20, SEQ ID NO: 21, and SEQ ID NO: 22, respectively, and
wherein the CALR binding domain and CD3 binding domain comprise a light chain variable region comprising a light chain CDR1 (LCDR1), light chain CDR2 (LCDR2), and light chain CDR3 (LCDR3), comprising an amino acid sequence as set forth in SEQ ID NO: 24, SEQ ID NO: 25, and SEQ ID NO: 26, respectively, or a light chain CDR1 (LCDR1), light chain CDR2 (LCDR2), and light chain CDR3 (LCDR3), comprising an amino acid sequence as set forth in SEQ ID NO: 27, SEQ ID NO: 28, and SEQ ID NO: 29, respectively.

In one embodiment, the present disclosure provides a bispecific binding moiety comprising:
a CALR binding domain comprising a heavy chain CDR1 (HCDR1), heavy chain CDR2 (HCDR2), and heavy chain CDR3 (HCDR3), having an amino acid sequence as set forth in SEQ ID NO: 10, SEQ ID NO: 11, and SEQ ID NO: 12, respectively, or a heavy chain CDR1 (HCDR1), heavy chain CDR2 (HCDR2), and heavy chain CDR3 (HCDR3), having an amino acid sequence as set forth in SEQ ID NO: 13, SEQ ID NO: 14, and SEQ ID NO: 15, respectively; and
a CD3 binding domain comprising a heavy chain CDR1 (HCDR1), heavy chain CDR2 (HCDR2), and heavy chain CDR3 (HCDR3), having an amino acid sequence as set forth in SEQ ID NO: 17, SEQ ID NO: 18, and SEQ ID NO: 19, respectively, or a heavy chain CDR1 (HCDR1), heavy chain CDR2 (HCDR2), and heavy chain CDR3 (HCDR3), having an amino acid sequence as set forth in SEQ ID NO: 20, SEQ ID NO: 21, and SEQ ID NO: 22, respectively, and
wherein the CALR binding domain and CD3 binding domain comprise a light chain variable region comprising a light chain CDR1 (LCDR1), light chain CDR2 (LCDR2), and light chain CDR3 (LCDR3), comprising an amino acid sequence as set forth in SEQ ID NO: 24, SEQ ID NO: 25, and SEQ ID NO: 26, respectively, or a light chain CDR1 (LCDR1), light chain CDR2 (LCDR2), and light chain CDR3 (LCDR3), comprising an amino acid sequence as set forth in SEQ ID NO: 27, SEQ ID NO: 28, and SEQ ID NO: 29, respectively.

In one embodiment, the present disclosure provides a bispecific binding moiety comprising a CALR binding domain and a CD3 binding domain, wherein the CALR binding domain comprises a heavy chain variable region having the amino acid sequence as set forth in SEQ ID NO: 1, or a heavy chain variable region that has at least 80%, at least 85%, at least 90%, or at least 95% sequence identity thereto, and the CD3 binding domain comprises a heavy chain variable region having the amino acid sequence as set forth in SEQ ID NO: 16, or a heavy chain variable region that has at least 80%, at least 85%, at least 90%, or at least 95% sequence identity thereto. In certain embodiments, each of the heavy chain variable regions comprise HCDRs that do not comprise amino acid variations. In certain embodiments, each of the heavy chain variable regions do not comprise amino acid variations.

In one embodiment, the present disclosure provides a bispecific binding moiety comprising a CALR binding domain and a CD3 binding domain, wherein the CALR binding domain comprises a heavy chain variable region having the amino acid sequence as set forth in SEQ ID NO: 8, or a heavy chain variable region that has at least 80%, at least 85%, at least 90%, or at least 95% sequence identity thereto, and the CD3 binding domain comprises a heavy chain variable region having the amino acid sequence as set forth in SEQ ID NO: 16, or a heavy chain variable region that has at least 80%, at least 85%, at least 90%, or at least 95% sequence identity thereto. In certain embodiments, each of the heavy chain variable regions comprise HCDRs that do not comprise amino acid variations. In certain embodiments, each of the heavy chain variable regions do not comprise amino acid variations.

In one embodiment, the present disclosure provides a bispecific binding moiety comprising a CALR binding domain and a CD3 binding domain, wherein the CALR binding domain comprises a heavy chain variable region having the amino acid sequence as set forth in SEQ ID NO: 9, or a heavy chain variable region that has at least 80%, at least 85%, at least 90%, or at least 95% sequence identity thereto, and the CD3 binding domain comprises a heavy chain variable region having the amino acid sequence as set forth in SEQ ID NO: 16, or a heavy chain variable region that has at least 80%, at least 85%, at least 90%, or at least 95% sequence identity thereto. In certain embodiments, each of the heavy chain variable regions comprise HCDRs that do not comprise amino acid variations. In certain embodiments, each of the heavy chain variable regions do not comprise amino acid variations.

In one embodiment, the present disclosure provides a bispecific binding moiety comprising a CALR binding domain and a CD3 binding domain, wherein the CALR binding domain comprises a heavy chain having the amino acid sequence as set forth in SEQ ID NO: 63, and the CD3 binding domain comprises a heavy chain having the amino acid sequence as set forth in SEQ ID NO: 66.

In one embodiment, the present disclosure provides a bispecific binding moiety comprising a CALR binding domain and a CD3 binding domain, wherein the CALR binding domain comprises a heavy chain having the amino acid sequence as set forth in SEQ ID NO: 64, and the CD3 binding domain comprises a heavy chain having the amino acid sequence as set forth in SEQ ID NO: 66.

In one embodiment, the present disclosure provides a bispecific binding moiety comprising a CALR binding domain and a CD3 binding domain, wherein the CALR binding domain comprises a heavy chain having the amino acid sequence as set forth in SEQ ID NO: 65, and the CD3 binding domain comprises a heavy chain having the amino acid sequence as set forth in SEQ ID NO: 66.

In one embodiment, the present disclosure provides a bispecific binding moiety comprising a CALR binding domain and a CD3 binding domain, wherein the CALR binding domain comprises a heavy chain variable region having the amino acid sequence as set forth in SEQ ID NO: 1, or a heavy chain variable region that has at least 80%, at least 85%, at least 90%, or at least 95% sequence identity thereto, the CD3 binding domain comprises a heavy chain variable region having the amino acid sequence as set forth in SEQ ID NO: 16, or a heavy chain variable region that has at least 80%, at least 85%, at least 90%, or at least 95% sequence identity thereto, and both the CALR binding domain and the CD3 binding domain comprise a light chain variable region having the amino acid sequence as set forth in SEQ ID NO: 23, or a light chain variable region that has at least 80%, at least 85%, at least 90%, or at least 95% sequence identity thereto. In certain embodiments, each of the heavy chain variable regions and light chain variable regions comprise HCDRs and LCDRs, respectively, that do not comprise amino acid variations. In certain embodiments, the each of the heavy chain variable regions and light chain variable regions do not comprise amino acid variations.

In one embodiment, the present disclosure provides a bispecific binding moiety comprising a CALR binding domain and a CD3 binding domain, wherein the CALR binding domain comprises a heavy chain variable region having the amino acid sequence as set forth in SEQ ID NO: 8, or a heavy chain variable region that has at least 80%, at least 85%, at least 90%, or at least 95% sequence identity thereto, the CD3 binding domain comprises a heavy chain variable having the amino acid sequence as set forth in SEQ ID NO: 16, or a heavy chain variable region that has at least 80%, at least 85%, at least 90%, or at least 95% sequence identity thereto, and both the CALR binding domain and the CD3 binding domain comprise a light chain variable region having the amino acid sequence as set forth in SEQ ID NO: 23, or a light chain variable region that has at least 80%, at least 85%, at least 90%, or at least 95% sequence identity thereto. In certain embodiments, each of the heavy chain variable regions and light chain variable regions comprise HCDRs and LCDRs, respectively, that do not comprise amino acid variations. In certain embodiments, the each of the heavy chain variable regions and light chain variable regions do not comprise amino acid variations.

In one embodiment, the present disclosure provides a bispecific binding moiety comprising a CALR binding domain and a CD3 binding domain, wherein the CALR binding domain comprises a heavy chain variable region having the amino acid sequence as set forth in SEQ ID NO: 9, or a heavy chain variable region that has at least 80%, at least 85%, at least 90%, or at least 95% sequence identity thereto, the CD3 binding domain comprises a heavy chain variable having the amino acid sequence as set forth in SEQ ID NO: 16, or a heavy chain variable region that has at least 80%, at least 85%, at least 90%, or at least 95% sequence identity thereto, and both the CALR binding domain and the CD3 binding domain comprise a light chain variable region having the amino acid sequence as set forth in SEQ ID NO: 23, or a light chain variable region that has at least 80%, at least 85%, at least 90%, or at least 95% sequence identity thereto. In certain embodiments, each of the heavy chain variable regions and light chain variable regions comprise HCDRs and LCDRs, respectively, that do not comprise amino acid variations. In certain embodiments, the each of the heavy chain variable regions and light chain variable regions do not comprise amino acid variations.

In one embodiment, the present disclosure provides a bispecific binding moiety comprising a CALR binding domain and a CD3 binding domain, wherein the CALR binding domain comprises a heavy chain having the amino acid sequence as set forth in SEQ ID NO: 63, the CD3 binding domain comprises a heavy chain having the amino acid sequence as set forth in SEQ ID NO: 66, and both the CALR binding domain and the CD3 binding domain comprise a light chain variable region having the amino acid sequence as set forth in SEQ ID NO: 60.

In one embodiment, the present disclosure provides a bispecific binding moiety comprising a CALR binding domain and a CD3 binding domain, wherein the CALR binding domain comprises a heavy chain having the amino acid sequence as set forth in SEQ ID NO: 64, the CD3 binding domain comprises a heavy chain having the amino acid sequence as set forth in SEQ ID NO: 66, and both the CALR binding domain and the CD3 binding domain comprise a light chain variable region having the amino acid sequence as set forth in SEQ ID NO: 60.

In one embodiment, the present disclosure provides a bispecific binding moiety comprising a CALR binding domain and a CD3 binding domain, wherein the CALR binding domain comprises a heavy chain having the amino acid sequence as set forth in SEQ ID NO: 65, the CD3 binding domain comprises a heavy chain having the amino acid sequence as set forth in SEQ ID NO: 66, and both the CALR binding domain and the CD3 binding domain comprise a light chain variable region having the amino acid sequence as set forth in SEQ ID NO: 60.

In certain embodiments, the present disclosure provides a CALR binding domain, wherein the CALR binding domain competes with a bispecific binding moiety as described herein for binding to CALR wildtype and/or mutant CALR.

In certain embodiments, the present disclosure provides a bispecific binding moiety that binds CALR and CD3, wherein the bispecific binding moiety competes with a bispecific binding moiety as described herein for binding to CALR wildtype and/or mutant CALR, and/or for binding to CD3.

For the purpose of the present disclosure, “compete”, “competes”, or “competing” refers to an activity of a binding domain or binding moiety that displaces a binding domain or binding moiety as described herein from its target antigen, in a cross-blocking assay. Therefore, in certain embodiments, a binding domain that competes for binding with the binding domain as described herein, binds to human CALR and displaces the binding domain as described herein, in a cross-blocking assay. In certain embodiments, a binding moiety that competes for binding with the binding moiety as described herein, binds to human CALR and human CD3 and displaces the binding moiety as described herein, in a cross-blocking assay. In certain embodiments, a cross-blocking assay is a competitive ELISA. Methods of performing a competitive ELISA are known to a person of ordinary skill in the art.

In brief, in a competitive ELISA, antigen is coated on the wells of a microtiter plate and pre-incubated with or without the competing binding moiety. This is followed by addition of a biotin-labeled binding domain or binding moiety as described herein. The amount of labeled binding domain or binding moiety bound to the antigen in the wells is measured using avidin-peroxidase conjugate and appropriate substrate. The amount of labeled biding domain or binding moiety that is bound to the antigen has an indirect correlation to the ability of the competing binding domain or binding moiety to compete for binding to the same antigen, i.e., the greater the affinity of the competing binding domain or binding moiety for the same antigen, the less labeled binding domain or binding moiety will be bound to the antigen-coated wells. A candidate competing binding domain or binding moiety is considered to compete for binding to the antigen, if the candidate binding domain or binding moiety can block binding of the binding domain or binding moiety of the present disclosure, to the target antigen, by at least 20%, or by at least 20-50%, or by at least 50%, as compared to the control performed in parallel in the absence of the candidate competing binding domain or binding moiety.

### Light Chains

In certain embodiments, a CALR binding domain, a CD3 binding domain, or a bispecific binding moiety of the present disclosure comprises a light chain variable region comprising the light chain CDR1 (LCDR1), light chain CDR2 (LCDR2), and light chain CDR3 (LCDR3) of a light chain variable region having the amino acid sequence as set forth in SEQ ID NO: 23. In certain embodiments, each of the LCDRs may comprise at most three, two, or one amino acid variations. In certain embodiments, only one of the LCDR1 or LCDR2 may comprise at most three, two, or one amino acid variations. In certain embodiments, LCDR3 does not comprise any amino acid variations. In certain embodiments, the amino acid variation is a conservative amino acid substitution.

In certain embodiments, the light chain variable region comprises light chain CDR1 (LCDR1), light chain CDR2 (LCDR2), and light chain CDR3 (LCDR3), having an amino acid sequence as set forth in SEQ ID NO. 24, SEQ ID NO: 25, and SEQ ID NO: 26, respectively, or light chain CDR1 (LCDR1), light chain CDR2 (LCDR2), and light chain CDR3 (LCDR3), having an amino acid sequence as set forth in SEQ ID NO: 27, SEQ ID NO: 28, and SEQ ID NO: 29, respectively. In certain embodiments, each of the LCDRs may comprise at most three, two, or one amino acid variations. In certain embodiments, only one of the LCDR1 or LCDR2 may comprise at most three, two, or one amino acid variations. In certain embodiments, LCDR3 does not comprise any amino acid variations. In certain embodiments, the amino acid variation is a conservative amino acid substitution.

In certain embodiments, the light chain variable region has the amino acid sequence as set forth in SEQ ID NO: 23, or having at least 80%, 85%, 90%, or 95% sequence identity thereto.

In certain embodiments, a CALR binding domain, CD3 binding domain, or bispecific binding moiety of the present disclosure also comprises variants, which, in addition to the variations in the heavy chain variable region and/or LCDRs referred to above, comprise one or more variations in the light chain variable framework regions. A variation can be any type of amino acid variation described herein, such as for instance a conservative amino acid substitution or non-conservative amino acid substitution resulting from somatic hypermutation or affinity maturation. In certain embodiments, a CALR binding domain, CD3 binding domain, or bispecific binding moiety variant of the present disclosure comprises no variations in the LCDR regions but comprises one or more variations in the framework regions. Such variants have at least 80%, or at least 85%, or at least 90%, or at least 95% sequence identity to the sequences disclosed herein, and are expected to retain binding specificity. Thus, in certain embodiments, a CALR binding domain, CD3 binding domain, or bispecific binding moiety of the present disclosure comprises:
a light chain variable region having at least 80%, or at least 85%, or at least 90%, or at least 95% sequence identity to the amino acid sequence as set forth in SEQ ID NO: 23, which light chain variable region comprises a LCDR1 amino acid sequence as set forth in SEQ ID NO: 24; a LCDR2 amino acid sequence as set forth in SEQ ID NO: 25; and a LCDR3 amino acid sequence as set forth in SEQ ID NO: 26, or a LCDR1 amino acid sequence as set forth in SEQ ID NO: 27; a LCDR2 amino acid sequence as set forth in SEQ ID NO: 28; and a LCDR3 amino acid sequence as set forth in SEQ ID NO: 29.

The CALR binding domain and the binding domains of the bispecific binding moiety of the present disclosure have been generated with a common light chain, in particular with a common light chain referred to as VK1-39/JK1. The CALR binding domain and the binding domains of the bispecific binding moiety of the present disclosure can; however, comprise any suitable light chain, including but not limited to common light chains known in the art. Examples of common light chains known in the art include, but are not limited to: VK1-39/JK5, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 32. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 32, wherein each of the LCDRs may comprise at most three, two, or one amino acid variations, for example substitutions. In certain embodiments, the light chain comprises a light chain variable region having an amino acid sequence as set forth in SEQ ID NO: 32, or having at least 80%, at least 85%, at least 90%, or at least 95% sequence identity thereto. In certain embodiments, the light chain comprises a light chain CDR1 (LCDR1), light chain CDR2 (LCDR2), and light chain CDR3 (LCDR3) having an amino acid sequence as set forth in SEQ ID NO: 24, SEQ ID NO: 25, and SEQ ID NO: 33, according to IMGT, respectively. In certain embodiments, the light chain comprises a light chain CDR1 (LCDR1), light chain CDR2 (LCDR2), and light chain CDR3 (LCDR3) according to Kabat, which are as underlined in the amino acid sequence as set forth in SEQ ID NO: 32; VK3-15/JK1, comprising a light chain variable region comprising a light chain CDR1 (LCDR1), light chain CDR2 (LCDR2), and light chain CDR3(LCDR3), of a light chain variable region having an amino acid sequence as set forth in SEQ ID NO: 35. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 35, wherein each of the LCDRs may comprise at most three, two, or one amino acid variations, for example substitutions. In certain embodiments, the light chain comprises a light chain variable region having an amino acid sequence as set forth in SEQ ID NO: 35, or having at least 80%, at least 85%, at least 90%, or at least 95% sequence identity thereto. In certain embodiments, the light chain comprises a light chain CDR1 (LCDR1), light chain CDR2 (LCDR2), and light chain CDR3 (LCDR3) having an amino acid sequence as set forth in SEQ ID NO: 36, SEQ ID NO: 37, and SEQ ID NO: 38, respectively. In certain embodiments, the light chain comprises a light chain CDR1 (LCDR1), light chain CDR2 (LCDR2), and light chain CDR3 (LCDR3) according to Kabat, which are as underlined in the amino acid sequence as set forth in SEQ ID NO: 35; VK3-20/JK1, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 40. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 40, wherein each of the LCDRs may comprise at most three, two, or one amino acid variations, for example substitutions. In certain embodiments, the light chain comprises a light chain variable region having an amino acid sequence as set forth in SEQ ID NO: 40, or having at least 80%, at least 85%, at least 90%, or at least 95% sequence identity thereto. In certain embodiments, the light chain comprises a light chain CDR1 (LCDR1), light chain CDR2 (LCDR2), and light chain CDR3 (LCDR3) having an amino acid sequence as set forth in SEQ ID NO: 41, SEQ ID NO: 42, and SEQ ID NO: 43, respectively. In certain embodiments, the light chain comprises a light chain CDR1 (LCDR1), light chain CDR2 (LCDR2), and light chain CDR3 (LCDR3) according to Kabat, which are as underlined in the amino acid sequence as set forth in SEQ ID NO: 40; and VL3-21/JL3, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 45. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 45, wherein each of the LCDRs may comprise at most three, two, or one amino acid variations, for example substitutions. In certain embodiments, the light chain comprises a light chain variable region having an amino acid sequence as set forth in SEQ ID NO: 45, or having at least 80%, at least 85%, at least 90%, or at least 95% sequence identity thereto. In certain embodiments, the light chain comprises a light chain CDR1 (LCDR1), light chain CDR2 (LCDR2), and light chain CDR3 (LCDR3) having an amino acid sequence as set forth in SEQ ID NO: 46, SEQ ID NO: 47, and SEQ ID NO: 48, respectively. In certain embodiments, the light chain comprises a light chain CDR1 (LCDR1), light chain CDR2 (LCDR2), and light chain CDR3 (LCDR3) according to Kabat, which are as underlined in the amino acid sequence as set forth in SEQ ID NO: 45.

VK1-39 is short for Immunoglobulin Variable Kappa 1-39 Gene. The gene is also known as immunoglobulin Kappa Variable 1-39; IGKV139; IGKV1-39; IgVκ1-39. External Ids for the gene are HGNC: 5740; Entrez Gene: 28930; Ensembl: ENSG00000242371. An amino acid sequence for VK1-39 is given as SEQ ID NO: 31. This is the sequence of the V-region. The V-region can be combined with one of five J-regions. Suitable VJ-region sequences are indicated as VK1-39/JK1 (SEQ ID NO: 23) and VK1-39/JK5 (SEQ ID NO: 32); alternative names are IgVκ1-39*01/IGJκ1*01 or IgVκ1-39*01/IGJκ5*01 (nomenclature according to the IMGT database worldwide web at imgt.org). These names are exemplary and encompass allelic variants of the gene segments.

VK3-15 is short for Immunoglobulin Variable Kappa 3-15 Gene. The gene is also known as Immunoglobulin Kappa Variable 3-15; IGKV315; IGKV3-15; IgVκ3-15. External Ids for the gene are HGNC: 5816; Entrez Gene: 28913; Ensembl: ENSG00000244437. An amino acid sequence for VK3-15 is given as SEQ ID NO: 34. This is the sequence of the V-region. The V-region can be combined with one of five J-regions. A suitable VJ-region sequence is indicated as VK3-15/JK1 (SEQ ID NO: 35); alternative name is Vκ3-15*01/IGJκ1*01 (nomenclature according to the IMGT database worldwide web at imgt.org). This name is exemplary and encompasses allelic variants of the gene segments.

VK3-20 is short for Immunoglobulin Variable Kappa 3-20 Gene. The gene is also known as Immunoglobulin Kappa Variable 3-20; IGKV320; IGKV3-20; IgVκ3-20. External Ids for the gene are HGNC: 5817; Entrez Gene: 28912; Ensembl: ENSG00000239951. An amino acid sequence for VK3-20 is indicated as SEQ ID NO: 39. This is the sequence of the V-region. The V-region can be combined with one of five J-regions. A suitable VJ-region sequence is indicated as VK3-20/JK1 (SEQ ID NO: 40); alternative name is IgVκ3-20*01/IGJκ1*01 (nomenclature according to the IMGT database worldwide web at imgt.org). This name is exemplary and encompasses allelic variants of the gene segments.

VL3-21 is short for Immunoglobulin Variable Lambda 3-21 Gene. The gene is also known as Immunoglobulin Lambda Variable 3-21; IGLV321; IGLV3-21; IgVλ3-21. External Ids for the gene are HGNC: 5905; Entrez Gene: 28796; Ensembl: ENSG00000211662.2. An amino acid sequence for VL3-21 is given as SEQ ID NO: 44. This is the sequence of the V-region. The V-region can be combined with one of five J-regions. A suitable VJ-region sequence is indicated as VL3-21/JL3 (SEQ ID NO: 45); alternative name is IgVλ3-21/IGJλ3 (nomenclature according to the IMGT database worldwide web at imgt.org). This name is exemplary and encompasses allelic variants of the gene segments.

Further, any light chain variable region of a CALR or CD3 antibody available in the art may be used, as may any other light chain variable region that can readily be obtained, such as from, for instance, an antibody display library by showing antigen binding activity when paired with an anti-CALR and anti-CD3 heavy chain variable as described herein.

In certain embodiments, the CALR binding domain and/or the CD3 binding domain of the present disclosure comprise a light chain having the amino acid sequence as set forth in SEQ ID NO: 60.

### Constant Regions

In certain embodiments, a CALR binding domain and/or CD3 binding domain of the present disclosure further comprises a CH1 region. In certain embodiments, a CALR binding domain and/or CD3 binding domains of the present disclosure further comprises a CH1 region, hinge, CH2 region, and CH3 region. A suitable CH1 region includes, but is not limited to, the CH1 region of which the amino acid sequence is set forth in SEQ ID NO: 50. A suitable hinge includes, but is not limited to, the hinge of which the amino acid sequence is set forth in SEQ ID NO: 49. Suitable CH2 and CH3 regions include, but are not limited to, the CH2 region of which the amino acid sequence is set forth in SEQ ID NO: 51 or 52 and the CH3 region of which the amino acid sequence is set forth in SEQ ID NO: 53, or 54 and 55. In certain embodiments, a CALR binding domain and/or CD3 binding domain of the present disclosure further comprises a CL region. A suitable CL region includes, but is not limited to, the CL region of which the amino acid sequence is set forth in SEQ ID NO: 30.

A CL, CH1, hinge, CH2, and/or CH3 region may be modified according to methods known in the art in order to obtain favorable antibody characteristics, including for instance to promote heterodimerization of different heavy chains, to improve heavy-light chain pairing, and to enhance or reduce immune cell effector function. A CH3 region may comprise the terminal lysine residue, or lack the terminal lysine residue to improve manufacturability.

Constant regions of a binding moiety of the present disclosure may comprise one or more variations that modulate properties of the binding moiety other than its binding properties to the target antigens or epitopes. For instance, the constant regions may comprise one or more variations that favor heterodimerization of the CALR and CD3 heavy chains over homodimerization of two CALR heavy chains and/or two CD3 heavy chains. Suitable variations that favor heterodimerization of two different heavy chains are, but are not limited to, those described for instance in WO 2013/157953 or WO 2013/157954. Also, the constant regions may comprise one or more variations that reduce or improve effector function, preferably one or more variations that reduce effector function. Further, the constant regions may comprise one or more variations that facilitate separation of the bispecific binding moiety from a mixture wherein it is produced. Suitable variations that facilitate separation of the bispecific binding moiety from a mixture wherein it is produced are, but are not limited to, those described for instance in WO 2020/226502.

### Nucleic Acids, Vectors, and Cells

In certain embodiments, the present disclosure provides a nucleic acid comprising a nucleic acid sequence that encodes a polypeptide or a heavy chain variable region as described herein. In certain embodiments, a nucleic acid of the present disclosure further comprises a nucleic acid sequence encoding a CH1 region. In certain embodiments, a nucleic acid of the present disclosure further comprises a nucleic acid sequence encoding a CH1 region, a hinge, CH2 region, and CH3 region. In certain embodiments, a nucleic acid of the present disclosure may further comprise at least one nucleic acid sequence encoding a light chain variable region, or encoding a light chain variable region and a CL region. In certain embodiments, the light chain variable region can be a light chain variable region as described herein.

In certain embodiments, the present disclosure provides a vector comprising a nucleic acid as described herein. In certain embodiments, a vector of the present disclosure comprises a nucleic acid sequence that encodes a polypeptide or heavy chain variable region as described herein and a CH1 region. In certain embodiments, a vector of the present disclosure encodes a polypeptide or heavy chain variable regions as described herein, a CH1 region, hinge, CH2 region, and CH3 region. In certain embodiments, a vector of the present disclosure further comprises a nucleic acid sequence that encodes a light chain variable (VL) region, or a light chain variable region and a light chain constant (CL) region. In certain embodiments, the light chain variable region is a light chain variable region of a light chain that is capable of pairing with multiple heavy chains having different epitope specificities.

In certain embodiments, the present disclosure provides a cell comprising a nucleic acid sequence encoding the heavy chain variable region of a CALR binding domain as described herein. In certain embodiments, the present disclosure provides a cell comprising a nucleic acid sequence encoding the heavy chain variable region of a CALR binding domain as described herein and a nucleic acid sequence encoding the heavy chain variable region of a CD3 binding domain as described herein. In certain embodiments, the cell further comprises a nucleic acid sequence encoding a CH1 region. In certain embodiment, the cell further comprises a nucleic acid sequence encoding a CH1 region, a hinge, CH2 region, and CH3 region. In certain embodiments, a cell of the present disclosure further comprises at least one nucleic acid sequence encoding a light chain variable (VL) region, or a light chain variable region and a light chain constant (CL) region. In certain embodiments, the light chain variable region is a light chain variable region of a light chain that is capable of pairing with multiple heavy chains having different epitope specificities.

In certain embodiments, the present disclosure provides a cell producing a bispecific binding moiety as described herein. In certain embodiments, the cell is a recombinant cell comprising a vector as described herein.

### Pharmaceutical Compositions and Methods of Use

In certain embodiments, the present disclosure provides a pharmaceutical composition comprising an effective amount of a binding moiety comprising a CALR binding domain as described herein, and a pharmaceutically acceptable carrier. In certain embodiments, the binding moiety comprising a CALR binding domain is an antibody, such as for instance an IgG1 antibody.

In certain embodiments, the present disclosure provides a pharmaceutical composition comprising an effective amount of a bispecific binding moiety as described herein, and a pharmaceutically acceptable carrier. In certain embodiments, the bispecific binding moiety is an antibody, such as for instance an IgG1 antibody.

In certain embodiment, the present disclosure provides a binding moiety comprising a CALR binding domain as described herein, a bispecific binding moiety as described herein, or a pharmaceutical composition as described herein, for use in therapy. In certain embodiments, the binding moiety comprising a CALR binding domain is an antibody, such as for instance an IgG1 antibody. In certain embodiments, the bispecific binding moiety is an antibody, such as for instance an IgG1 antibody. In certain embodiments, the binding moiety comprising a CALR binding domain or the bispecific binding moiety, or a pharmaceutical composition comprising the same, is for use in the treatment of cancer.

In certain embodiments, the present disclosure provides a binding moiety comprising a CALR binding domain as described herein, a bispecific binding moiety as described herein, or a pharmaceutical composition as described herein, for use in the treatment of myeloproliferative neoplasms (MPN).

In certain embodiments, the present disclosure provides a binding moiety comprising a CALR binding domain as described herein, a bispecific binding moiety as described herein, or a pharmaceutical composition as described herein, for use in the treatment of essential thrombocythemia (ET).

In certain embodiments, the present disclosure provides a binding moiety comprising a CALR binding domain as described herein, a bispecific binding moiety as described herein, or a pharmaceutical composition as described herein, for use in the treatment of myelofibrosis (MF).

In certain embodiments, the binding moiety comprising a CALR binding domain is an antibody, such as for instance an IgG1 antibody. In certain embodiments, the bispecific binding moiety is an antibody, such as for instance an IgG1 antibody.

In certain embodiments, the present disclosure provides a method for treating a disease, comprising administering an effective amount of a binding moiety comprising a CALR binding domain as described herein, a bispecific binding moiety as described herein, or a pharmaceutical composition as described herein, to a subject in need thereof. In certain embodiments, the disease is cancer.

In certain embodiments, the present disclosure provides a method for treating a disease, comprising administering an effective amount of a binding moiety comprising a CALR binding domain as described herein, a bispecific binding moiety as described herein, or a pharmaceutical composition as described herein, to a subject in need thereof. In certain embodiments, the disease is a myeloproliferative neoplasm (MPN). In certain embodiments, the disease is essential thrombocythemia (ET). In certain embodiments, the disease is myelofibrosis (MF).

As used herein, the terms “individual”, “subject” and “patient” are used interchangeably and refer to a mammal such as a human, mouse, rat, hamster, guinea pig, rabbit, cat, dog, monkey, cow, horse, pig and the like, and in particular to a human subject having cancer.

The terms “treat,” “treating,” and “treatment,” as used herein, refer to any type of intervention or process performed on or administering an active agent or combination of active agents to a subject with the objective of curing or improving a disease or symptom thereof or which produces a positive therapeutic response. As used herein, “positive therapeutic response” refers to a treatment producing a beneficial effect, e.g. reversing, alleviating, ameliorating, inhibiting, or slowing down a symptom, complication, condition or biochemical indicia associated with a disease, as well as preventing the onset, progression, development, severity or recurrence of a symptom, complication, condition or biochemical indicia associated with a disease, such as, for example, amelioration of at least one symptom of a disease or disorder, e.g. cancer. A beneficial effect can take the form of an improvement over baseline, including an improvement over a measurement or observation made prior to initiation of therapy according to the method. For example, a beneficial effect can take the form of slowing, stabilizing, stopping or reversing the progression of a cancer in a subject at any clinical stage, as evidenced by a decrease or elimination of a clinical or diagnostic symptom of the disease, or of a marker of cancer. Effective treatment may, for example, decrease tumor size, decrease the presence of circulating tumor cells, reduce or prevent metastases of a tumor, slow or arrest tumor growth and/or prevent or delay tumor recurrence or relapse.

The term “therapeutic amount” or “effective amount” refers to an amount of an agent or combination of agents that treats a disease, such as cancer. In some embodiments, a therapeutic amount is an amount sufficient to delay tumor development. In some embodiments, a therapeutic amount is an amount sufficient to prevent or delay tumor recurrence.

As used herein, an effective amount of the agent or composition is one that, for example, may: (i) reduce the number of cancer cells; (ii) reduce tumor size; (iii) inhibit, retard, slow to some extent and may stop cancer cell infiltration into peripheral organs; (iv) inhibit tumor metastasis; (v) inhibit tumor growth; (vi) prevent or delay occurrence and/or recurrence of tumor; and/or (vii) relieve to some extent one or more of the symptoms associated with the cancer.

An effective amount may vary according to factors such as the disease state, age, sex, and weight of the individual to be treated, and the ability of the agent or combination of agents to elicit a desired response in the individual, which can be readily evaluated by the ordinarily skilled physician or other health care worker.

An effective amount can be administered to a subject in one or more administrations.

An effective amount can also include an amount that balances any toxic or detrimental effects of the agent or combination of agents and the beneficial effects.

The term “agent” refers to a therapeutically active substance, in the present case a multispecific binding moiety of the present disclosure, or a pharmaceutical composition of the present disclosure.

As used herein, “to comprise” and its conjugations is used in its non-limiting sense to mean that items following the word are included, but items not specifically mentioned are not excluded.

The articles “a” and “an” are used herein to refer to one or more of the grammatical object of the article. By way of example, “an element” means one or more elements.

A reference herein to a patent document or other matter is not to be taken as an admission that that document or matter was known or that the information it contains was part of the common general knowledge at the priority date of any of the claims.

All patent and literature references cited in the present specification are hereby incorporated by reference in their entirety.

CDRs and framework regions of antibodies have been described and defined in the art using a number of different systems, including for instance Kabat (see Sequences of Proteins of Immunological Interest (National Institute of Health, Bethesda, Md., 1987 and 1991), Kabat et al., J. Biol. Chem. 252:6609-6616 (1977)), IMGT (discussed in Giudicelli et al., Nucleic Acids Res. 25: 206-21 1 1997), Chothia (Chothia and Lesk J. Mol. Biol. 196: 901-917, 1987; Chothia et al., Nature 342: 877-883, 1989; Al-Lazikani et al., J. Mol. Biol. 273: 927-948, 1997), and the nomenclatures of Honnegher and Plukthun (Honnegher and Plukthun, J. Mol. Biol. 309: 657-670, 2001), MacCallum (MacCallum et al., J. Mol. Biol. 262:732-745 (1996); Abhinandan and Martin, Mol. Immunol., 45: 3832-3839 (2008)), and Lefranc (Lefranc M. P. et al., Dev. Comp. Immunol., 27: 55-77 (2003)). In general, any numbering system can be used considering that an antibody exhibits its properties regardless of which numbering system is used to define the CDRs. When the amino acid sequence of a variable region is given, a skilled person can readily determine its CDRs based on different numbering systems. Thus, the present disclosure encompasses defining the CDRs in accordance with each numbering system available to a skilled person. In particular, the present disclosure encompasses defining the CDRs in accordance with the numbering systems of Kabat, IMGT, and Chothia. In certain embodiments, the CDRs are as defined in Table 9 (table providing the CDRs according to Kabat and IMGT). Amino acids in the constant regions are indicated according to the EU numbering system. Accession numbers are primarily given to provide a further method of identification of a target, the actual sequence of the protein bound may vary, for instance because of a mutation in the encoding gene such as those occurring in some cancers or the like. An antigen binding site of a binding domain or binding moiety of the disclosure can bind the antigen and a variety of variants thereof, such as those expressed by some antigen positive immune or tumor cells. HGNC stands for the HUGO Gene nomenclature committee. The number following the abbreviation is the accession number with which information on the gene and protein encoded by the gene can be retrieved from the HGNC database. Entrez Gene provides the accession number or gene ID with which information on the gene or protein encoded by the gene can be retrieved from the NCBI (National Center for Biotechnology Information) database. Ensembl provides the accession number with which information on the gene or protein encoded by the gene can be obtained from the Ensembl database. Ensembl is a joint project between EMBL-EBI and the Wellcome Trust Sanger Institute to develop a software system which produces and maintains automatic annotation on selected eukaryotic genomes.

When herein reference is made to a gene or a protein, the reference is preferably to the human form of the gene or protein. When herein reference is made to a gene or protein reference is made both to the natural gene or protein and to variant forms of the gene or protein as can be detected in tumors, cancers and the like, preferably as can be detected in human tumors, cancers and the like.

### BRIEF DESCRIPTION OF THE DRAWINGS

The following naming conventions are used herein as follows. In the Figures, bivalent bispecific antibodies are indicated in the format Fab x target, where “Fab” refers to an anti-CALR binding domain comprising a particular heavy chain variable region and a light chain variable region, and “target” refers to the target of the second binding domain comprising a heavy chain variable region and a light chain variable region. Each binding domain of the bispecific antibodies comprises the same light chain as described herein.

Bivalent bispecific antibodies are also indicated in the format BsAb, where “BsAb” refers to a bispecific antibody comprising a particular anti-CALR binding domain and a particular CD3 binding domain. Each binding domain of the bispecific antibodies comprises the same light chain as described herein.

FIG. 1A-FIG. 1I—Antibody binding to wildtype human CALR analyzed by ELISA. RSV and CALRwt refer to the negative and positive control antibodies, respectively. Antibodies were tested at different concentrations, depicted on the x-axis. OD values measured at 450 nm are shown on the y-axis.

FIG. 2A-FIG. 2C—T cell cytotoxicity assay using TF1 as target cells which are edited to express human CALRmut del52 (FIG. 2B) and CALRmut ins5 (FIG. 2C), and unedited parental TF1 cells (FIG. 2A), analyzed by FACS. Antibodies were tested at different concentrations, depicted on the x-axis. For percentage cytotoxicity, cells were gated as total cells, followed by CFSE+ tumor cells, and dead cells, as shown on the y-axis.

FIG. 3A-FIG. 3C—T cell cytotoxicity assay using PLB-985 as target cells which are edited to express human CALRmut del52 (FIG. 3B) and CALRmut ins5 (FIG. 3C), and unedited parental PLB-985 cells (FIG. 3A), analyzed by FACS. Antibodies were tested at different concentrations, depicted on the x-axis. For percentage cytotoxicity, cells were gated as total cells, followed by CFSE+ tumor cells, and dead cells, as shown on the y-axis.

FIG. 4—Antibody internalization into human T cells measured by Incucyte Live Cell analysis. Internalization of antibodies was measured every hour over 24 h, as depicted on the x-axis, by brightfield and fluorescence (550 nm) scans. The area of cells fluorescing red due to internalization is expressed as percentage of total cell area in the well (y-axis).

FIG. 5A-FIG. 5C—T cell cytotoxicity assay using TF1 as target cells which are edited to express human CALRmut del52 (FIG. 5B) and CALRmut ins5 (FIG. 5C), and unedited parental TF1 cells (FIG. 5A), analyzed by FACS. Mock refers to the TTxCD3 control antibody. Antibodies were tested at different concentrations, depicted on the x-axis. For percentage cytotoxicity, cells were gated as total cells, followed by CFSE+ tumor cells, and dead cells, as shown on the y-axis.

FIG. 6—Schematic representation of Cryo-EM data. Fab region (Fab Y) of bivalent monospecific antibody Ab Y, with identical CALR binding domain as BsAb 18, was used to identify the domain of the CALRmut protein to which it binds. Fab region (Fab X) of monoclonal antibody Ab X, known to bind to the C-terminus of CALRmut, was used as comparison.

FIG. 7A-FIG. 7C—Flow cytometry-based cell binding assay using TF1 parental cells (FIG. 7A), TF1 cells expressing CALRmut del52 (FIG. 7B) and TF1 cells expressing CALRmut ins5 (FIG. 7C). Cells were incubated with different concentrations of antibodies, depicted on the x-axis. Binding was measured and expressed as gMFI, as shown on the y-axis. Mean value of two replicates is shown, and the error bars represent SD.

FIG. 8A-FIG. 8C—Flow cytometry-based cell binding assay using Ba/F3 parental cells (FIG. 8A), Ba/F3 cells expressing CALRmut del52 (FIG. 8B) and Ba/F3 cells expressing CALRmut ins5 (FIG. 8C). Cells were incubated with different concentrations of antibodies as depicted on the x-axis. Binding was measured and expressed as gMFI shown on the y-axis. Mean value of two replicates is shown, and the error bars represent SD.

FIG. 9A-FIG. 9B—Flow cytometry-based binding assay of BsAb 18 to primary CD34⁺ cells from a healthy donor (FIG. 9A) or a myelofibrosis donor (FIG. 9B). Antibody concentration is shown on the x-axis and antibody binding expressed as gMFI is shown on the y-axis. Mean value of two replicates is shown, and the error bars represent SD.

FIG. 10—Dose-response curves showing binding kinetics of antibodies to surface translocated wild-type calreticulin in Reh cells treated with doxorubicin. Geometric mean fluorescence intensity (gMFI) is shown on the y-axis. Antibody concentration is shown on the x-axis.

FIG. 11A-FIG. 11D—Binding of BsAb 18 to TF-1 CALRmut ins5 cells after pre-incubation with patient plasma (FIG. 11A) or healthy donor plasma (FIG. 11B). Binding of mock control IgG1 antibody (TTxCD3) to TF-1 CALRmut ins5 cells after pre-incubation with patient plasma (FIG. 11C) or healthy donor plasma (FIG. 11D). Binding is expressed as gMFI on the y-axis for serially diluted bispecific antibodies (shown on x-axis) which were pre-incubated with multiple concentrations of plasma (0% to 40%). gMFI was also measured using PE-secondary staining (secondary only) for each group.

FIG. 12A-FIG. 12B—Binding of BsAb 18 and mock control IgG1 antibody (TTxCD3) to TF-1 CALRmut del52 cells (FIG. 12A) or TF1 CALRmut ins5 cells (FIG. 12B). Cells were incubated with different concentrations of soluble CALR (without pre-incubation) followed by addition of serially diluted antibodies (as shown on x-axis). Binding is expressed as gMFI on the y-axis.

FIG. 13A-FIG. 13J—T cell cytotoxicity assay using TF1 parental cells (FIG. 13A, FIG. 13D, FIG. 13G), TF1 cells expressing CALRmut del52 (FIG. 13B, FIG. 13E, FIG. 13H, FIG. 13J) and CALRmut ins5 (FIG. 13C, FIG. 13F, FIG. 131) as target cells in a TF1 and T cell co-culture assay. Antibodies (mock control IgG1 antibody (TT×CD3), negative control anti-RSV antibody, vibecotamab, Ab X or BsAb 18) were tested at different concentrations, depicted on the x-axis. For percentage cytotoxicity, cells were gated as total cells/CFSE+ tumor cells/dead cells, shown on the y-axis. The graph shows % cytotoxicity from a T cell Donor 1 (FIG. 13A-C), Donor 2 (FIG. 13D-F), Donor 3 (FIG. 13G-I), Donor 4 (FIG. 13J). Mean value of two replicates is shown and error bars represent SD.

FIG. 14A-FIG. 14C—T cell activation assay using TF1 parental cells (FIG. 14A), TF1 cells expressing CALRmut del52 (FIG. 14B) and CALRmut ins5 (FIG. 14C) as target cells in a TF1 and T cell co-culture assay. Antibodies (mock control IgG1 antibody (TTxCD3), negative control anti-RSV antibody, vibecotamab, Ab X or BsAb 18) were tested at different concentrations, depicted on the x-axis. For percentage T cell activation shown on the y-axis, cells were gated as total cells/CFSE negative T cells/live cells/CD69 positive cells. The graph shows % activation from a representative T cell donor. Mean value of two replicates is shown and error bars represent SD.

FIG. 15A-FIG. 15F—T cell proliferation assay using TF1 parental cells (FIG. 15A, FIG. 15D), TF1 cells expressing CALRmut del52 (FIG. 15B, FIG. 15E) and CALRmut ins5 (FIG. 15C, FIG. 15F) as target cells in a TF1 and T cell co-culture assay. Antibodies (mock control IgG1 antibody (TTxCD3), negative control anti-RSV antibody, vibecotamab, Ab X or BsAb 18) were tested at different concentrations, depicted on the x-axis. For percentage CD8+ T cell proliferation shown on the y-axis, cells were gated as total cells/Live cells/CD8+ T cells/Frequency of CFSE positive CD8 T cells. The graph shows % proliferation on the y-axis for T cell donor 1 (FIG. 15A-C) and donor 2 (FIG. 15D-F). Mean value of two replicates is shown and error bars represent SD.

FIG. 16A-FIG. 16L—T-cell cytokine release assay in a co-culture of TF1 parental cells (FIG. 16A, FIG. 16D, FIG. 16G, and FIG. 16J), TF1 cells expressing CALRmut del52 (FIG. 16B, FIG. 16E, FIG. 16H, and FIG. 16K), CALRmut ins5 (FIG. 16C, FIG. 16F, FIG. 16I, and FIG. 16L) and T-cells from one representative T-cell donor. Antibodies (mock control IgG1 antibody (TT×CD3), vibecotamab, Ab X or BsAb 18) were tested at different concentrations, depicted on x-axis. Cytokine levels (pg/mL) for the indicated cytokines are shown on the y-axis. Mean value of two replicates is shown and error bars represent SD. FIG. 16A-C: Cytokine IFN-γ; FIG. 16 D-F: TNFα; FIG. 16G-I: IL-6; FIG. 16 J-L: IL-10.

FIG. 17A-FIG. 17D—BsAb 18-mediated T cell activation and T cell cytotoxicity against MF patient derived CD34+ cells. MF patient derived CD34+ cells expressing CALRmut del52 and CALRmut ins5 were labeled with the CFSE stain and co-cultured with healthy donor T cells in the presence of serially diluted antibodies. FIG. 17A-B: T cells from cocultures were evaluated for activation by the expression of CD25 on CD8+ T cells by flow cytometry when co-cultured with MF patient derived CD34+ cells expressing CALRmut del52 (FIG. 17A) and CALRmut ins5 (FIG. 17B). FIG. 17C-D: Cytotoxicity was assessed by identifying dead target cells by gating CFSE+/Live-Dead+ stained cells. FIG. 17C: MF patient with CALRmut del52; FIG. 17D: MF patient with CALRmut type2.

FIG. 18A-FIG. 18D—Cytokine analysis after incubation of antibodies with healthy donor T cells. Each data point is the average across corresponding duplicates. Antibodies (mock control IgG1 antibody (TT×CD3), negative control IgG1 antibody (RSV), vibecotamab, mosunetuzumab or BsAb 18) were tested at different concentrations, depicted on the x-axis. Cytokine levels (pg/mL) for the indicated cytokines are shown on the y-axis. Cytokine levels are shown for a representative T cell donor (1 out of 5 tested) for IFN-γ (FIG. 18A), TNF-α (FIG. 18B), IL-6 (FIG. 18C), and IL-10 (FIG. 18D).

FIG. 19A-FIG. 19D—Cytokine analysis after incubation of antibodies with healthy donor PBMCs. Each data point is the average across corresponding duplicates. Antibodies (mock control IgG1 antibody (TT×CD3), negative control IgG1 antibody (RSV), vibecotamab, mosunetuzumab or BsAb 18) were tested at different concentrations, depicted on the x-axis. Cytokine levels (pg/mL) for the indicated cytokines are shown on the y-axis. Cytokine levels are shown for a representative T cell donor (1 out of 5 tested) for IFN-γ (FIG. 19A), TNF-α (FIG. 19B), IL-6 (FIG. 19C), and IL-10 (FIG. 19D).

FIG. 20A-FIG. 20D—Cytokine analysis incubation of antibodies with PBMCs isolated from patients carrying CALRmut del52. Antibodies (mock control IgG1 antibody (TTxCD3), negative control IgG1 antibody (RSV), vibecotamab, mosunetuzumab or BsAb 18) were tested at different concentrations, depicted on the x-axis. Cytokine levels (pg/mL) for the indicated cytokines are shown on the y-axis. Each data point is the average across corresponding duplicates. Cytokine levels are shown for a representative PBMC donor (1 out of 5 donors tested) for IFN-γ (FIG. 20A), TNF-α (FIG. 20B), IL-6 (FIG. 20C), and IL-10 (FIG. 20D).

FIG. 21A-FIG. 21D—Cytokine analysis after incubation of antibodies with PBMCs isolated from patients carrying CALRmut ins5. Antibodies (mock control IgG1 antibody (TT×CD3), negative control IgG1 antibody (RSV), vibecotamab, mosunetuzumab or BsAb 18) were tested at different concentrations, depicted on the x-axis. Cytokine levels (pg/mL) for the indicated cytokines are shown on the y-axis. Each data point is the average across corresponding duplicates. Cytokine levels are shown for one representative PBMC donor, for IFN-γ (FIG. 21A), TNF-α (FIG. 21 B), IL-6 (FIG. 21C), IL-10 (FIG. 21D).

### EXAMPLES

In the Examples, which are used to illustrate the present disclosure but are not intended to limit the disclosure in any way, each binding domain of the bispecific antibodies comprises a light chain variable region having an amino acid sequence as set forth in SEQ ID NO: 23 and a light chain constant region having an amino acid sequence as set forth in SEQ ID NO: 30. The bispecific antibodies preferably are IgG1 antibodies comprising a CH1, hinge, CH2, and CH3.

In certain embodiments, each binding domain of the bispecific antibodies comprise a light chain having an amino acid sequence as set forth in SEQ ID NO: 60.

In the Examples, which are used to illustrate the present disclosure but are not intended to limit the disclosure in any way, bispecific antibodies were screened in IgG1 format, wherein the CALR binding heavy chain comprises a CH1 having an amino acid sequence as set forth in SEQ ID NO: 50, a CH2 having an amino acid sequence as set forth in SEQ ID NO: 52, and a CH3 having an amino acid sequence as set forth in SEQ ID NO: 54; and the CD3 binding heavy chain comprises a CH1 having an amino acid sequence as set forth in SEQ ID NO: 50, a CH2 having an amino acid sequence as set forth in SEQ ID NO: 52, and a CH3 having an amino acid sequence as set forth in SEQ ID NO: 55.

BsAbs 1-18 are all bivalent bispecific antibodies comprising a CALR binding domain and CD3 binding domain.

BsAb 16 is a bivalent bispecific antibody comprising a heavy chain variable region having an amino acid sequence as set forth in SEQ ID NO: 1, a heavy chain variable region having an amino acid sequence as set forth in SEQ ID NO: 16, and two light chain variable regions having an amino acid sequence as set forth in SEQ ID NO: 23.

BsAb 17 is a bivalent bispecific antibody comprising a heavy chain variable region having an amino acid sequence as set forth in SEQ ID NO: 9, a heavy chain variable region having an amino acid sequence as set forth in SEQ ID NO: 16, and two light chain variable regions having an amino acid sequence as set forth in SEQ ID NO: 23.

BsAb 18 is a bivalent bispecific antibody comprising a heavy chain variable region having an amino acid sequence as set forth in SEQ ID NO: 8, a heavy chain variable region having an amino acid sequence as set forth in SEQ ID NO: 16, and two light chain variable regions having an amino acid sequence as set forth in SEQ ID NO: 23. Reference antibodies and molecules, and control antibodies used in the Examples include:
Reference antibody analog vibecotamab, which is a human bivalent recombinant monospecific antibody (Thermo Fisher Scientific, #PIMA542248).
Positive control antibody CAL2 (CAL2), which is a mouse anti-mutant human CALR antibody (Dianova, DIA-CAL-250).
Positive control antibody CALRwt (CALRwt), which is a rabbit polyclonal bivalent monospecific antibody binding human CALRwt (Abcam, #ab2907)
Negative control IgG1 antibody (RSV), which is a bivalent monospecific antibody comprising two heavy chains having an amino acid sequence as set forth in SEQ ID NO: 59 and two light chains having an amino acid sequence as set forth in SEQ ID NO: 60.
Negative control IgG1 antibody (TT), which is a bivalent monospecific antibody comprising two heavy chains having an amino acid sequence as set forth in SEQ ID NO: 61 and two light chains having an amino acid sequence as set forth in SEQ ID NO: 60.
Mock control IgG1 antibody (TT×CD3; mock CD3), which is a bivalent bispecific antibody comprising a binding domain that binds TT and a representative binding domain that binds CD3.
Antibody X (Ab X) is an antibody known to bind to the C-terminus of mutant CALR.
Antibody Y (Ab Y) is a bivalent monospecific antibody comprising two heavy chains having an amino acid sequence as set forth in SEQ ID NO: 8 and two light chains having an amino acid sequence as set forth in SEQ ID NO: 23.
Antibody Z is an antibody binding wtCALR.
Reference antibody analog mosunetuzumab, is a CD20×CD3 bispecific antibody (RefDrug Lunsumio National Drug Code #50242-159-01; lot M2993B23; Hillsborough, NJ).

### Example 1—Generation of Binding Domains

Heavy chain variable regions with binding specificity to human Calreticulin (CALR) and heavy chain variable regions with binding specificity to human CD3 were obtained by immunizing transgenic mice comprising a common IGKV1-39 light chain (MeMo® mice) with human CALR or CD3 antigenic moieties, including the use of different forms of DNA, peptide, protein and cell-based antigen delivery.

Bivalent monospecific antibodies and bivalent bispecific antibodies comprising the CALR binding heavy chain variable regions were produced and characterized for binding specificity and affinity.

### FACS

The antibodies were tested for binding to mutant human CALR, including CALRmut type 1 (del52), which has a deletion of the amino acid at position 52, and type 2 (ins5), which has an insertion at position 5. 293F cells were transiently transfected with CALRmut del52 and MPL, CALRmut ins5 and MPL, or mock-transfected, and analyzed by FACS. All antibodies were tested at 5 μg/ml, including the positive control antibody mouse anti-mutant human CALR (CAL2, Dianova, DIA-CAL-250), and negative control IgG1 antibody (RSV). All antibodies were detected with F(ab′)2-Goat anti-Human IgG Fc Secondary Antibody, PE (Invitrogen, #H10104) at 1:100 dilution in FACS buffer. FACS buffer contained the following: PBS (pH7.4, Gibco, #10010-015), 0.5% BSA (SigmaAldrich, #A3294), and 2 mM EDTA (0.5M, pH 8.0, UltraPure, Invitrogen, #15575-020). Cells were first harvested by centrifuging for 5 min at 300 g at 4° C. and counted. Supernatant was discarded and the pellet was resuspended in ice-cold FACS buffer at a concentration of 1×10⁶ cells/ml. 50.000-200.000 cells were plated per well in a U-bottom 96-well FACS plate (BD, #353910) and centrifuged for 3 min at 300 g at 4° C. (Eppendorf 5810R with rotor A-4-62). Supernatant was discarded. 50 μl of primary antibody solution was added to the cells and mixed. The cells were then incubated for 30 min at 2-8° C. in the dark. The cells were then washed by adding 150 μl ice-cold FACS buffer and centrifuged for 3 min at 300 g at 4° C. Supernatant was discarded and cells were washed again by adding 200 μl ice-cold FACS buffer and centrifuged for 3 min at 300 g at 4° C. Supernatant was discarded afterwards. 50 μl secondary antibody solution was added to the cells and mixed. The cells were incubated for 30 min at 2-8° C. in the dark. Cells were then washed by adding 150 μl ice-cold FACS buffer and centrifuged for 3 min at 300 g at 4° C. The supernatant was discarded and cells were washed again by adding 200 μl ice-cold FACS buffer and centrifuged for 3 min at 300 g at 4° C. Supernatant was discarded and cells were resuspended in 120 μl FACS buffer. Cells were analyzed with iQue VBR (Intellicyt) or FACS Fortessa (BD).

### ELISA

The antibodies were further analyzed for their binding to human CALR wildtype (CALRwt) with ELISA. ELISA plates (Greiner Bio-One, #655061) were coated with 1 μg/ml CALRwt protein (Bio-connect, #LS-G97217) 50 μl/ml overnight at 4° C. The plates were then washed twice with wash buffer PBS/T (PBS (pH7.4, Gibco, #10010-015)+10% (v/v) Tween 20 (Merck, #8.22184.0500)) using ELISA washer (BioTek 405TS). The wells were then blocked for 1 hour at RT with 300 μl/well block buffer (PBS (pH7.4, Gibco, #10010-015)+5% BSA (Sigma. #A3294-500g)). All antibodies, including negative control IgG1 antibody (RSV), were tested at an 8-step 4× dilution starting at 10 μg/ml in block buffer. Rabbit polyclonal CALRwt antibody (Abcam, #ab2907) was taken along as control with 1:50 starting dilution. After blocking, the plates were emptied, and 50 μl of antibodies were added to the wells. The plates were covered with seal (EASYseal, Greiner, #676001) and incubated for 60 min at RT. Afterwards, the plates were washed three times with wash buffer using ELISA washer. All antibodies were detected with 501p/well secondary antibody mouse anti-human IgG HRP-conjugated (BD, #555788) at 1:2000 dilution, except for the control CALRwt antibody, which was detected by Peroxidase-AffiniPure Donkey Anti-Rabbit IgG (H+L) (Jackson ImmunoResearch, #711-035-152) at 1:2000 dilution. The plates were covered with EASYseal and incubated for 60 min at RT. Plates were then washed three times with wash buffer using ELISA washer. TMB Substrate solution (BD OptEIA™ TMB Substrate Reagent Set, BD, #555214) was prepared by mixing reagents A and B in 1:1 ratio and added to the wells with 50 μl/well. The plates were developed for maximal 10 min and the reaction was stopped by adding 50 μl/well of 1 M H₂SO₄(95-97%≈18 M. Merck, #1.00731). The plates were analyzed using ELISA plate reader (BioTek ELx808) at A450.

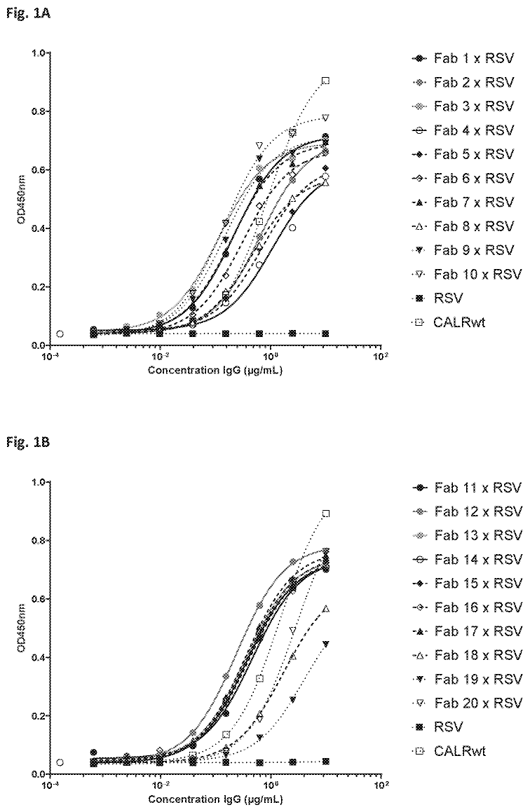

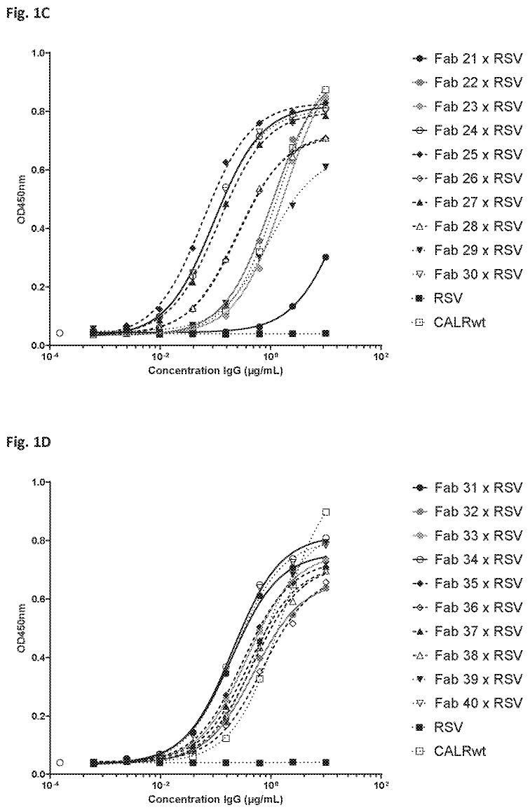

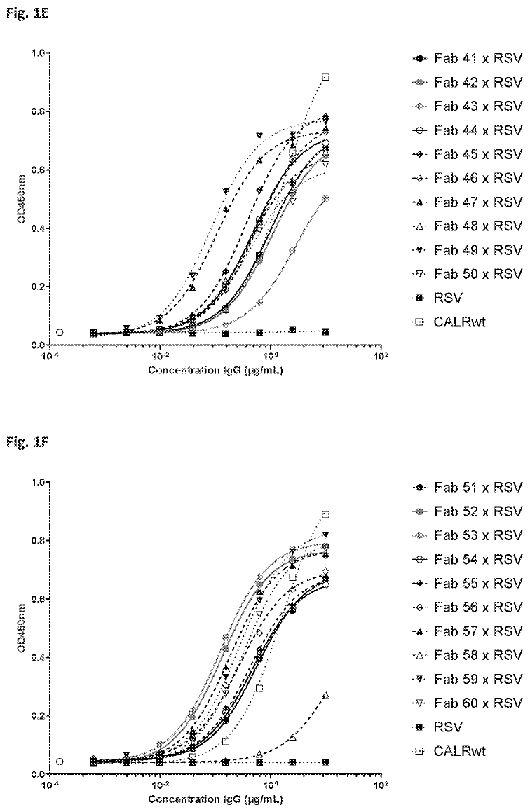

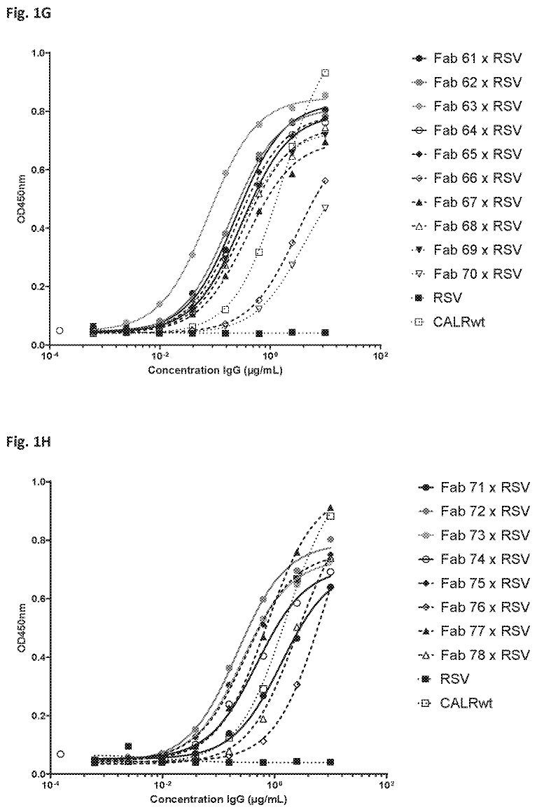

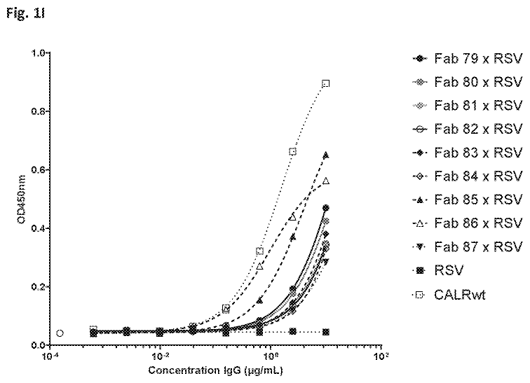

As shown in FIG. 1 (ELISA) and Table 1 (FACS), the antibodies bind to human CALR wildtype (WT) as well as to human CALRmut del52 and human CALRmut ins5.

The antibodies show a variety in binding characteristics (Table 1): a number of antibodies show a similar level of binding to CALRmut del52 and CALRmut ins5; a number of antibodies show relative higher levels of binding to CALRmut ins5 as compared to CALRmut del52; a number of antibodies show relative lower, similar, or higher levels of binding to CALRmut del52 and/or CALRmut ins5 as compared to the control antibody CAL2. The antibody comprising Fab25, comprising a heavy chain variable region having an amino acid sequence as set forth in SEQ ID NO: 1, has a relative higher level of binding to CALRmut del52 as compared to the control antibody CAL2 and a similar level of binding to CALRmut ins5. The antibody comprising Fab53, comprising a heavy chain variable region having an amino acid sequence as set forth in SEQ ID NO: 9, has a similar level of binding to CALRmut del52 as compared to the control antibody CAL2 and a slightly higher level of binding to CALRmut ins5.

The antibodies show a variety in binding characteristics to CALRwt (FIG. 1): a number of antibodies show a relative higher level of binding than control antibody CALRwt. The antibody comprising Fab25, comprising a heavy chain variable region having an amino acid sequence as set forth in SEQ ID NO: 1, has a significantly higher level of binding to CALRwt as compared to the control antibody CALRwt. The antibody comprising Fab53, comprising a heavy chain variable region having an amino acid sequence as set forth in SEQ ID NO: 9, has a relative higher level of binding to CALRwt as compared to the control antibody CALRwt.

TABLE 1
Antibody binding to 293F cells that were transiently
transfected with CALRmut del52 and MPL,
CALRmut ins5 and MPL, or mock-transfected.
Mean fluorescence intensity (MFI) was measured by FACS.

| | CALRmut | CALRmut | |
|---|---|---|---|
| | del52 | ins5 | Mock |
| | (MFD) | (MFI) | (MFI) |
| Fab 1 × RSV | 59336 | 109016 | 5593 |
| Fab 2 × RSV | 38247 | 50201 | 5919 |
| Fab 3 × RSV | 67876 | 134363 | 5692 |
| Fab 4 × RSV | 35975 | 61428 | 5772 |
| Fab 5 × RSV | 47372 | 106132 | 5786 |
| Fab 6 × RSV | 49566 | 76737 | 5647 |
| Fab 7 × RSV | 53250 | 98990 | 5452 |
| Fab 8 × RSV | 53730 | 119769 | 5463 |
| Fab 9 × RSV | 75656 | 134950 | 5796 |
| Fab 10 × RSV | 66038 | 126984 | 6091 |
| Fab 11 × RSV | 56757 | 91596 | 5906 |
| Fab 12 × RSV | 75766 | 111646 | 5904 |
| Fab 13 × RSV | 64909 | 102661 | 5852 |
| Fab 14 × RSV | 66349 | 115658 | 5712 |
| Fab 15 × RSV | 61460 | 91681 | 5786 |
| Fab 16 × RSV | 65933 | 110456 | 5654 |
| Fab 17 × RSV | 68573 | 118110 | 5942 |
| Fab 18 × RSV | 46602 | 88183 | 6221 |
| Fab 19 × RSV | 27375 | 69081 | 5957 |
| Fab 20 × RSV | 71359 | 78675 | 6187 |
| Fab 21 × RSV | 23166 | 36947 | 5939 |
| Fab 22 × RSV | 60261 | 88474 | 6085 |
| Fab 23 × RSV | 53520 | 81302 | 5947 |
| Fab 24 × RSV | 96563 | 154629 | 6218 |
| Fab 25 × RSV | 106222 | 183872 | 6182 |
| Fab 26 × RSV | 61712 | 121156 | 5971 |
| Fab 27 × RSV | 84758 | 139305 | 6106 |
| Fab 28 × RSV | 66387 | 116249 | 6061 |
| Fab 29 × RSV | 58815 | 115663 | 5952 |
| Fab 30 × RSV | 75090 | 189056 | 5871 |
| Fab 31 × RSV | 55376 | 128174 | 5730 |
| Fab 32 × RSV | 51801 | 107738 | 6180 |
| Fab 33 × RSV | 30909 | 85735 | 6118 |
| Fab 34 × RSV | 76387 | 155535 | 6008 |
| Fab 35 × RSV | 62163 | 97690 | 6169 |
| Fab 36 × RSV | 49131 | 113182 | 6140 |
| Fab 37 × RSV | 54894 | 122589 | 5905 |
| Fab 38 × RSV | 49665 | 112039 | 5996 |
| Fab 39 × RSV | 49018 | 88696 | 5869 |
| Fab 40 × RSV | 81526 | 141122 | 6282 |
| Fab 41 × RSV | 28575 | 48231 | 6232 |
| Fab 42 × RSV | 27324 | 70179 | 5975 |
| Fab 43 × RSV | 17670 | 40197 | 5972 |
| Fab 44 × RSV | 57235 | 113471 | 5938 |
| Fab 45 × RSV | 65862 | 148609 | 5972 |
| Fab 46 × RSV | 42924 | 101984 | 5861 |
| Fab 47 × RSV | 83617 | 169653 | 6148 |
| Fab 48 × RSV | 61087 | 136786 | 6245 |
| Fab 49 × RSV | 84863 | 178826 | 5979 |
| Fab 50 × RSV | 49918 | 134464 | 5912 |
| Fab 51 × RSV | 51021 | 145032 | 6013 |
| Fab 52 × RSV | 86033 | 168979 | 5981 |
| Fab 53 × RSV | 86119 | 174191 | 5936 |
| Fab 54 × RSV | 52506 | 148658 | 5801 |
| Fab 55 × RSV | 64877 | 145074 | 6533 |
| Fab 56 × RSV | 66415 | 142923 | 6202 |
| Fab 57 × RSV | 76765 | 172759 | 6022 |
| Fab 58 × RSV | 16416 | 56348 | 5875 |
| Fab 59 × RSV | 64065 | 154929 | 6187 |
| Fab 60 × RSV | 54431 | 115072 | 6013 |
| Fab 61 × RSV | 48160 | 136768 | 5987 |
| Fab 62 × RSV | 56208 | 174375 | 5807 |
| Fab 63 × RSV | 110376 | 212469 | 6141 |
| Fab 64 × RSV | 79483 | 152742 | 6248 |
| Fab 65 × RSV | 77060 | 154593 | 6039 |
| Fab 66 × RSV | 14075 | 35772 | 5994 |
| Fab 67 × RSV | 51683 | 145319 | 6031 |
| Fab 68 × RSV | 61338 | 167152 | 5965 |
| Fab 69 × RSV | 62317 | 165698 | 5836 |
| Fab 70 × RSV | 26606 | 85128 | 6256 |
| Fab 71 × RSV | 46958 | 139008 | 6187 |
| Fab 72 × RSV | 87738 | 168327 | 6824 |
| Fab 73 × RSV | 65374 | 144510 | 6031 |
| Fab 74 × RSV | 51062 | 141473 | 6106 |
| Fab 75 × RSV | 47229 | 139325 | 5974 |
| Fab 76 × RSV | 44390 | 120648 | 6120 |
| Fab 77 × RSV | 77275 | 170413 | 6113 |
| Fab 78 × RSV | 85173 | 179657 | 6315 |
| Fab 79 × RSV | 48444 | 72902 | 6018 |
| Fab 80 × RSV | 47276 | 64603 | 6280 |
| Fab 81 × RSV | 39801 | 56973 | 6224 |
| Fab 82 × RSV | 31119 | 38274 | 6187 |
| Fab 83 × RSV | 30805 | 35637 | 6200 |
| Fab 84 × RSV | 25453 | 34816 | 5906 |
| Fab 85 × RSV | 51005 | 81567 | 5901 |
| Fab 86 × RSV | 37457 | 107073 | 6029 |
| Fab 87 × RSV | 27163 | 41034 | 5988 |
| RSV | 7298 | 7449 | 6190 |
| CAL2 | 66877 | 129406 | 6206 |
| Secondary Ab | 6758 | 7145 | 6111 |
| No Ab | 6505 | 6829 | 5868 |

A large panel of CALR×CD3 bispecific antibodies combining different CALR and CD3 binding domains was generated by transient co-transfection of two plasmid vectors: one encoding an IgG heavy chain with a CALR binding VH region and the other encoding an IgG heavy chain with a CD3 binding VH region. CH3 engineering technology as described in WO 2013/157954 and WO 2013/157953 was employed to ensure efficient hetero-dimerization and formation of bispecific antibodies. Both vectors further encode a common light chain comprising the IGKV1-39/Jk1 light chain variable region. Cell transfection, cell culture, and the harvesting and purification of antibodies was performed by methods known in the art. Further CH3 engineering technologies, for instance as described in WO 2021/235936, may be employed to ensure efficient dimerization and formation of bispecific antibodies.

### Example 2

The panel of CALR×CD3 bispecific antibodies was characterized in a T cell cytotoxicity assay using BaF3 as target cells which were edited to express human CALRmut and MPL.

BaF3 cells (DSMZ, Germany) were seeded with a density of 20.000 cells per well in RPMI640 with 10% human serum. The bispecific antibodies were tested in 3 concentrations; 9000 ng/ml, 900 ng/ml, and 90 ng/ml. Mock control IgG1 antibody (TT×CD3) and negative control IgG1 antibody (TT) were included at 6-step 5-fold dilutions starting at 9 μg/ml and 3-step 5-fold dilutions starting at 9 μg/ml, respectively. After adding the antibody dilutions to the target cells, the cells were incubated for 30 minutes at RT. Unstimulated T cells were used as effector cells with a 5-1 E:T ratio. After adding the T cells to the target cells, the cells were allowed to settle for 30 minutes at RT, followed by an incubation of 72 hours at 37° C. with 5% CO₂. The cells were then incubated with CytoTox96® Non-Radioactive Cytotoxicity Assay Kit (Promega, cat #G1980) for 30 minutes at RT followed by the stop solution. Lactate dehydrogenase (LDH), a stable cytosolic enzyme that is released upon cell lysis, was measured on the EnVision® Multilabel Plate Reader (Revvity) at 485 nm absorbance within 1 hour after the stop solution was added. Percentage of target cell lysis was calculated as follows: % cytotoxicity=experimental well−no antibody control (avg.)/max lysis (avg.)−no antibody control (avg.)×100. Data and AUC were analyzed by using GraphPad Prism software (version 10). Data is shown in Table 2. Based on this data, a number of CALR×CD3 bispecific antibodies were selected for further characterization.

TABLE 2
T cell cytotoxicity assay of BaF3 target cells.
Area under the curve (AUC) and AUC fold change
over relevant mock control (TT × CD3).

| | | AUC fold |
|---|---|---|
| | | change |
| Antibodies | AUC | over mock |
| BsAb 1 | 40.5 | 5.01 |
| BsAb 2 | 39.95 | 5.57 |
| BsAb 3 | 47.65 | 6.64 |
| BsAb 4 | 38.95 | 4.38 |
| BsAb 5 | 33.85 | 4.69 |
| BsAb 6 | 31.8 | 3.89 |
| BsAb 7 | 30.75 | 13.42 |
| BsAb 8 | 35.55 | 69.06 |
| BsAb 9 | 26.3 | 55.50 |
| BsAb 10 | 16.25 | 35.86 |
| BsAb 11 | 10.9 | 17.26 |
| BsAb 12 | 18.15 | 32.17 |
| BsAb 13 | 18.6 | 33.15 |
| BsAb 14 | 23.75 | 38.60 |
| BsAb 15 | 22.95 | 41.04 |
| BsAb 16 | 35.25 | 63.13 |
| BsAb 17 | 29.2 | 54.94 |
| Mock CD3 | 2.595 | 1.00 |

### Example 3

The panel of CALR×CD3 bispecific antibodies was characterized in a T cell cytotoxicity assay using TF-1 cells as target cells engineered to express human CALRmut del52 and TF-1 cells engineered to express human CALRmut ins5.

TF1 parental cells were grown in RPMI 1640/10% HI-FBS/2 ng/ml GM-CSF. TF1 cells expressing human CALRmut del52 or CALRmut ins5 were grown in RPMI 1640/10% HI-FBS/2 ng/ml GM-CSF/0.5 ug/ml Puromycin media. Target cells were labeled with CFSE (Life Technologies, #C3454) according to manufacturer's protocol. CFSE-labeled target cells were then incubated with Fc block (Biolegend, cat #4422302) for 10 mins at RT. Bispecific antibodies were added at 12-step 5-fold dilutions, starting with 50 μg/ml, into designated wells followed by T cell addition at E:T ratio of 5:1. Cells were incubated for 72 hours at 37° C. with 5% CO₂. For percentage cytotoxicity, cells were analyzed by FACS and gated as total cells, followed by CFSE+ tumor cells, and dead cells. Data and AUC were analyzed by using GraphPad Prism software (version 10).

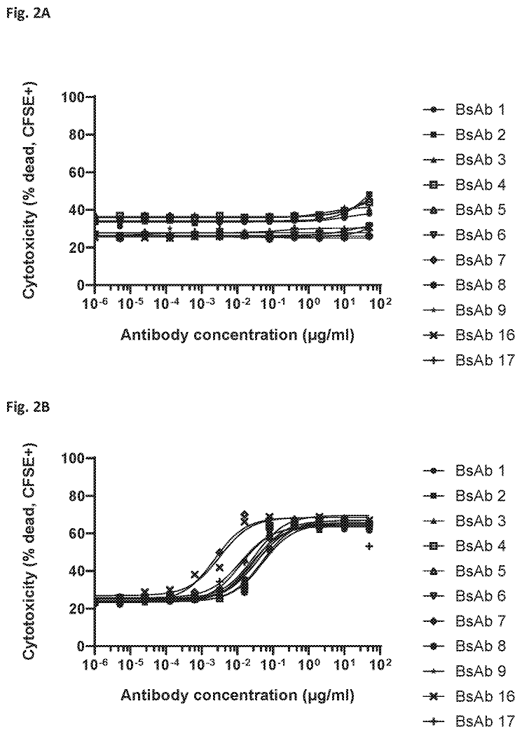

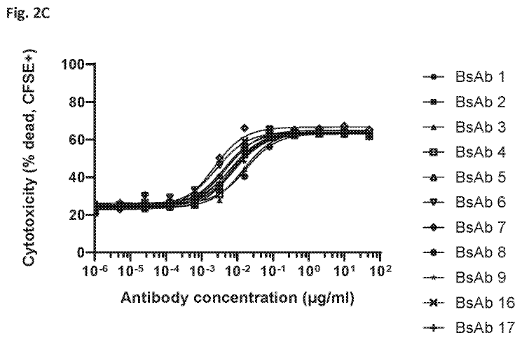

Data is shown in FIG. 2 and Table 3. All bispecific antibodies induced T cell mediated killing of TF1 cells expressing human CALRmut del52 and TF1 cells expressing CALRmut ins5 but not of TF1 parental cells.

TABLE 3
T cell cytotoxicity assay of TF1 parental, TF1 expressing
human CALRmut del52 or CALRmut ins5 target cells.
Area under the curve (AUC) was measured.

| | TF1 | TF1 | TF1 |
|---|---|---|---|
| | parental | CALRmut | CALRmut |
| Antibodies | AUC | del52 AUC | ins5 AUC |
| BsAb 1 | 1826 | 3099 | 3145 |
| BsAb 2 | 2098 | 3132 | 3122 |
| BsAb 3 | 2028 | 3184 | 3146 |
| BsAb 4 | 2054 | 3251 | 3128 |
| BsAb 5 | 2081 | 3216 | 3144 |
| BsAb 6 | 1478 | 3192 | 3163 |
| BsAb 7 | 1317 | 3330 | 3312 |
| BsAb 8 | 1269 | 3269 | 3135 |
| BsAb 9 | 1391 | 3377 | 3108 |
| BsAb 16 | 1406 | 3377 | 3182 |
| BsAb 17 | 1521 | 3004 | 3148 |

### Example 4

The panel of CALR×CD3 bispecific antibodies was characterized in a T cell cytotoxicity assay using PLB-985 cells as target cells engineered to express human CALRmut del52 and PLB-985 cells engineered to express human CALRmut ins5.

PLB-985 parental cells were grown in RPMI/10% HI-FBS whereas PLB-985 expressing human CALRmut del52 or CALRmut ins5 were grown in RPMI/10% HI-FBS/1 ug/ml Puromycin. Target cells were labeled with CFSE (Life Technologies, #C3454) according to manufacturer's protocol. CFSE-labeled target cells were then incubated with Fc block (Biolegend, cat #4422302) for 10 mins at RT. Bispecific antibodies were added at 8-step 5-fold dilutions, starting at 50 μg/ml, into designated wells followed by T cell addition at E:T ratio of 5:1. Cells were incubated for 72 hours at 37° C. with 5% CO2. For percentage cytotoxicity, cells were gated as total cells/CFSE+ tumor cells/dead cells. Data and AUC were analyzed by using GraphPad Prism software (version 10).

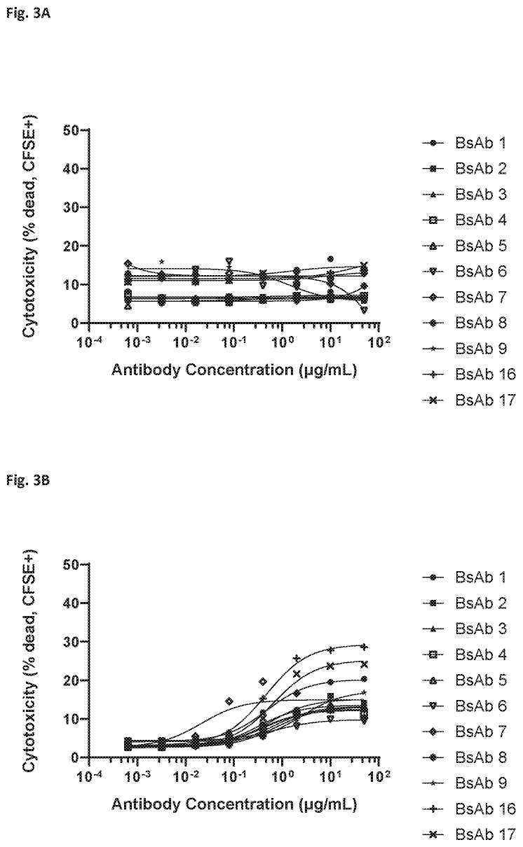

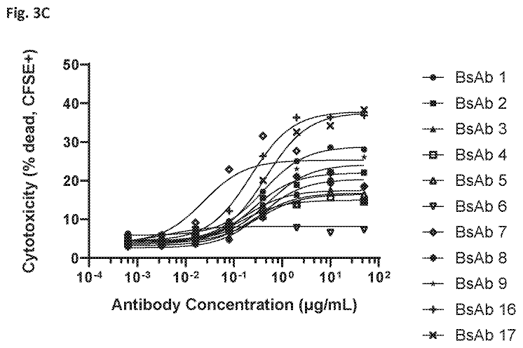

Data is shown in FIG. 3 and Table 4. All bispecific antibodies induced T cell mediated killing of PLB-985 cells expressing human CALRmut del52 and PLB-985 cells expressing CALRmut ins5 but not of PLB-985 parental cells. Bispecific antibodies 16 and 17 show the highest relative activity in this assay.

TABLE 4
T cell cytotoxicity assay of PLB-985 parental, PLB-985
expressing human CALRmut del52 or CALRmut ins5 target
cells. Area under the curve (AUC) was measured.

| | PLB-985 | PLB-985 | PLB-985 |
|---|---|---|---|
| | parental | CALRmut del52 | CALRmut ins5 |
| Antibodies | AUC | AUC | AUC |
| BsAb 1 | 365.2 | 652.7 | 871.0 |
| BsAb 2 | 315.4 | 730.9 | 1081 |
| BsAb 3 | 306.6 | 605.7 | 811.7 |
| BsAb 4 | 336.9 | 588.9 | 746.5 |
| BsAb 5 | 336.3 | 624.4 | 811.1 |
| BsAb 6 | 384.2 | 472.7 | 353.3 |
| BsAb 7 | 587.4 | 602.3 | 1066 |
| BsAb 8 | 374.5 | 605.9 | 947.0 |
| BsAb 9 | 330.1 | 737.7 | 1106 |
| BsAb 16 | 629.6 | 1377 | 1815 |
| BsAb 17 | 675.2 | 1167 | 1764 |

### Example 5

The panel of CALR×CD3 bispecific antibodies was characterized in an internalization assay.

Human T cells were isolated by negative selection from leukopaks collected from two healthy donors and stored at −80° C. until needed. For the assay, T cells were thawed, washed with culture medium (RPMI 1640 medium containing 10% fetal bovine serum) and allowed to rest for 15 minutes. 50,000 T cells were seeded into each well of a retronectin-coated, 96-well, clear plate (Corning #3596) and allowed to attach overnight at 37° C. in a 5% CO₂ incubator. CALR×CD3 bispecific antibodies were labeled with Incucyte human Fab Fluor pH antibody dye (Sartorius, #4722) according to the manufacturer's protocol. The dye used in these experiments fluoresce red in the acidic environment of the lysosome, indicating internalization of the labeled antibody. All antibodies were added to each well at a final concentration of 5 μg/mL. The plate was placed in an Incucyte S3 Live Cell Analysis Instrument (Sartorius, NY), scanned at 10× magnification immediately, then every hour for 24 h capturing brightfield and fluorescence at 550 nm with each scan. Brightfield images were used to determine proliferation as measured by the confluence of the well. Antibody internalization was calculated as the percentage of red cells among the cell population. Wells with cells in growth medium without antibody or Fab Fluor were used to monitor cell viability. Internalization was measured by quantifying the area of red cells as a percentage of total cell area in the well. Data and AUC were analyzed by using GraphPad Prism software (version 10).

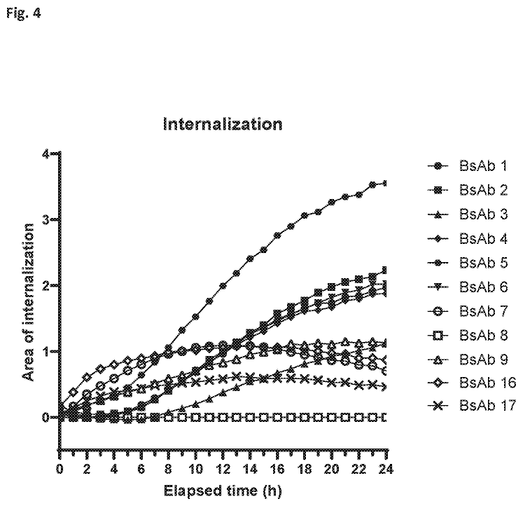

Results are shown in FIG. 4 and Table 5. Bispecific antibodies show a diverse range of activity in inducing internalization. Bispecific antibodies 16 and 17 show relative low rates of internalization compared to other bispecific antibodies.

TABLE 5
Bispecific antibodies internalization by human T
cells. Area under the curve (AUC) was measured.

| Antibodies | AUC |
|---|---|
| BsAb 1 | 22.56 |
| BsAb 2 | 24.48 |
| BsAb 3 | 10.27 |
| BsAb 4 | 21.96 |
| BsAb 5 | 44.77 |
| BsAb 6 | 22.89 |
| BsAb 7 | 19.97 |
| BsAb 8 | 0 |
| BsAb 9 | 18.03 |
| BsAb 16 | 21.94 |
| BsAb 17 | 11.54 |

### Example 6

CALR×CD3 bispecific antibodies were characterized in a cynomolgus monkey PK study. For in vivo pharmacokinetic experiments, antibodies were administered to male and female cynomolgus monkeys via 30 minutes intravenous infusion at 0.6 and 3 mg/kg (1 or 2 animals/antibody/dose). PBS buffer was used as dose vehicle. Blood samples were collected at 0.5, 1, 4, 12, 24, 48, 120, 168, 216, 336, 504 and 672 hours post-dose. All blood samples were allowed to clot and then centrifuged to obtain serum. The serum concentrations of antibodies were determined by Meso Scale Discovery biomarker assays. The measured serum concentrations were used to calculate PK parameters by standard noncompartmental methods using Phoenix® WinNonlin software program (version 8.3.4, Pharsight Corporation).

A High Bind MSD plate was coated with a goat anti-human IgG antibody in carbonate-bicarbonate buffer and allowed to incubate overnight at 4° C. The plates were washed and blocked with 1× casein in PBS for at least 1 hour with shaking. Calibration standards and quality control (QC) samples were prepared by spiking stock solutions of antibodies into pooled cynomolgus monkey serum. Prepared calibration standards, QC samples, and study monkey serum samples were diluted to a minimum required dilution (MRD) of 1:150 in 1× Casein in Wash Buffer A (PBS with 0.05% Tween-20) prior to loading onto the plates. Following washing of the plates, samples were then added to the blocked plates and incubated for 2 hours with shaking. The plates were then washed and biotinylated goat anti-human IgG antibody was added to the plate for 1 hour with shaking. Following plate washing, the plates were subsequently incubated with sulfo-tagged streptavidin. To determine the amount of bound analytes, the plates were washed and MSD Read Buffer T was added prior to the plates being read on an MSD Meso Sector Imager S 600. Upon electrochemical stimulation, the Sulfo-tag conjugate on the antibody complex emitted light on the electrode surfaces of the plate. Within the quantifiable range of the curve, the intensity of the light emitted, measured as electrochemiluminescence (ECL) units, was proportional to the concentration of antibodies present. The ECL units emitted by the calibration standards were fitted using a four-parameter logistic (4-PL) fit equation with 1/y² weighting to calculate the antibody concentrations in the QC and study samples. The recovery of the various antibodies was shown to be within ±30% based on the standard curve.

Non-compartmental analysis (NCA) of plasma concentration data was conducted using Phoenix® WinNonlin® software, version 8.3.4 (Certara). The NCA consistent with IV bolus dosing for the plasma data was used for parameters estimation (WinNonlin model 200-202). The following configuration was used for the analysis: Sampling Method—Serial, AUC Calculation Method—Linear Trapezoidal Linear Interpolation, Lambda Z (λZ) method—Best fit (R2) or time range for λZ Linear-Log regression, Nominal Dose Time—Set to zero. Clearance is calculated by WinNolin's non-compartmental analysis using equation Dose/AUCinf.

Results for two bispecific antibodies are shown in Table 6. Bispecific antibodies 16 and 17 show relative low clearance in this study.

TABLE 6
Clearance of bispecific antibodies in cynomolgus monkey.

| | Dose | Clearance |
|---|---|---|
| Antibodies | (mg/kg) | (ml/h/kg) |
| BsAb 16 | 0.6 | 0.222 |
| | 3 | 0.221 |
| BsAb 17 | 0.6 | 0.222 |
| | 3 | 0.179 |

### Example 7

CALR×CD3 bispecific antibodies were characterized in a T cell cytotoxicity assay using TF-1 cells as target cells which express human CALRmut del52 or human CALRmut ins5, repeating the assay as described in Example 3. Bispecific antibodies were added at 8-step 5-fold dilutions, starting with 50 μg/ml. Mock control IgG1 antibody (TT×CD3) was used as negative control. Data and AUC were analyzed by using GraphPad Prism software (version 10).

Results for three bispecific antibodies are shown in FIG. 5 and Table 7. All three bispecific antibodies induced T cell mediated killing of TF1 cells expressing human CALRmut del52 and TF1 cells expressing CALRmut ins5 but not of TF1 parental cells.

TABLE 7
T cell cytotoxicity assay of TF1 parental, TF1 expressing
human CALRmut del52 or CALRmut ins5 target cells.
Area under the curve (AUC) was measured.

| | TF1 parental | TF1 CALRmut | TF1 CALRmut |
|---|---|---|---|
| Antibodies | AUC | del52 AUC | ins5 AUC |
| BsAb 16 | 778.5 | 3050 | 3303 |
| BsAb 17 | 878.3 | 2479 | 3472 |
| BsAb 18 | 686.0 | 2994 | 3309 |
| Mock | 849.0 | 879.0 | 795.7 |
| Untreated | 523.9 | 416.3 | 498.8 |

### Example 8

CALR×CD3 bispecific antibodies were characterized in a PBMC cytokine release assay. TF-1 cells were used as target cells which express human CALRmut del52 or human CALRmut ins5. The TF-1 cells were grown in RPMI 1640/10% HI-FBS/2 ng/ml GM-CSF/0.5 ug/ml Puromycin media. PBMCs were maintained in RPMI/10% FBS (Cellular technologies Limited, cat #CTL-UP1). For CRS assay, target cells were added into round bottom 96-well plates, followed by 7-step 5-fold dilutions of bispecific antibodies starting at 50 μg/ml. Mock control IgG1 antibody (TT×CD3) and vibecotamab benchmark antibody were included as negative and positive control, respectively. PBMCs from healthy donor were added at 5:1 E:T ratio. Cells were incubated for 72 hours at 37° C. with 5% CO2. After 72 hours, supernatant was collected and cytokine analysis was carried out on the supernatants using Meso Scale Discovery biomarker assay according to manufacturer's protocol (MSD, V-plex Proinflammatory Panel 1 human kit, cat #K15049D-4). Data was analyzed using MSD Discovery workbench software (Meso Scale Diagnostics) and data was plotted using GraphPad Prism software (version 9). Results for 5 bispecific antibodies are shown in Table 8.

TABLE 8
Cytokine release assay of the co-culture of PBMCs and TFI
CALRmut del52 or CALRmut ins5 cells. Area under the curve
(AUC) was measured for IL-6, IL-10, and TNF-a.

| | TF-1 CALRmut del52 | | | TF-1 CALRmut ins5 | | |
|---|---|---|---|---|---|---|
| | IL-6 | IL-10 | TNF-a | IL-6 | IL-10 | TNF-a |
| | AUC | AUC | AUC | AUC | AUC | AUC |
| BsAb 7 | 13273 | 831.8 | 12457 | 15344 | 600.7 | 14147 |
| BsAb 9 | 360.8 | 448.6 | 882.9 | 588.9 | 381.6 | 2003 |
| BsAb 16 | 752.4 | 613.4 | 1233 | 886.8 | 353.3 | 2183 |
| BsAb 17 | 1780 | 352.7 | 1965 | 2891 | 263.2 | 3525 |
| Vibecotamab | 1354 | 2228 | 2621 | 1849 | 1462 | 4009 |
| Mock | 0 | 0.2682 | 3.087 | 0.2425 | 1.07 | 10.49 |

TABLE 9
HCDR sequences of bispecific antibody 16, comprising a CALR binding domain with a heavy chain
variable region having an amino acid sequence as set forth in SEQ ID NO: 1, and a CD3 binding domain with
a heavy chain variable region having an amino acid sequence as set forth in SEQ ID NO: 16; bispecific antibody
17, comprising a CALR binding domain with a heavy chain variable region having an amino acid sequence as set
forth in SEQ ID NO: 9, and a CD3 binding domain with a heavy chain variable region having an amino acid
sequence as set forth in SEQ ID NO: 16; bispecific antibody 18, comprising a CALR binding domain with a heavy
chain variable region having an amino acid sequence as set forth in SEQ ID NO: 8, and a CD3 binding domain with
a heavy chain variable region having an amino acid sequence as set forth in SEQ ID NO: 16, according to IMGT
and Kabat numbering systems. All binding domains comprise a light chain variable region having an amino acid
sequence as set forth in SEQ ID NO: 23. The LCDRs according to IMGT are as set forth in SEQ ID NOs: 24, 25,
and 26. The LCDRs according to Kabat are as set forth in SEQ ID NOs: 27, 28, and 29.

| | HCDR1 | HCDR2 | HCDR3 | HCDRI | HCDR2 | HCDR3 |
|---|---|---|---|---|---|---|
| | IMGT | IMGT | IMGT | Kabat | Kabat | Kabat |
| BsAb 16 | SEQ ID NO: 2 | SEQ ID NO: 3 | SEQ ID NO: 4 | SEQ ID NO: 5 | SEQ ID NO: 6 | SEQ ID NO: 7 |
| CALR | | | | | | |
| BsAb 16 | SEQ ID NO: 17 | SEQ ID NO: 18 | SEQ ID NO: 19 | SEQ ID NO: 20 | SEQ ID NO: 21 | SEQ ID NO: 22 |
| CD3 | | | | | | |
| BsAb 17 | SEQ ID NO: 10 | SEQ ID NO: 11 | SEQ ID NO: 12 | SEQ ID NO: 13 | SEQ ID NO: 14 | SEQ ID NO: 15 |
| CALR | | | | | | |
| BsAb 17 | SEQ ID NO: 17 | SEQ ID NO: 18 | SEQ ID NO: 19 | SEQ ID NO: 20 | SEQ ID NO: 21 | SEQ ID NO: 22 |
| CD3 | | | | | | |
| BsAb 18 | SEQ ID NO: 2 | SEQ ID NO: 3 | SEQ ID NO: 4 | SEQ ID NO: 5 | SEQ ID NO: 6 | SEQ ID NO: 7 |
| CALR | | | | | | |
| BsAb 18 | SEQ ID NO: 17 | SEQ ID NO: 18 | SEQ ID NO: 19 | SEQ ID NO: 20 | SEQ ID NO: 21 | SEQ ID NO: 22 |
| CD3 | | | | | | |

### Example 9

CalR×CD3 bispecific antibodies were characterized utilizing cryo-EM imaging to identify the domain of the CALRmut protein that is bound by the bispecific antibodies.

Cryo-EM grids were prepared by combining a complex of co-purified CALRmut ins5 and the Fab region of Ab X (Fab X), an antibody known to bind to the C-terminus of CALRmut, with the Fab region of Ab Y (Fab Y), a bivalent monospecific antibody binding to CALR, with identical CALR binding domain as BsAb 18. The final concentration of the sample was adjusted with PBS to 0.2 mg/ml of the total protein concentration and incubated for 30 minutes on ice. Next, the protein sample was applied to Quantifoil R1.2/1.3 200 mesh Cu grids (Quantifoil Micro Tools GmbH) and frozen using Vitrobot Mark IV set (Thermo Fisher Scientific Inc.) to 3 s blotting time, 10 force, 100% humidity and 4° C.

Cryo-EM images were collected on a 200 kV Glacios (GLACIOSTEM; Thermo Fisher Scientific Inc.) with a Falcon4i detector (FALCON4DETECTOR; Thermo Fisher Scientific Inc.) using EPU (Data collection Table). Images were corrected for gain reference and drift using MotionCorr (Zheng et al., 2017). Contrast transfer function (CTF) estimation was performed using CTFFIND4 (Rohou and Grigorieff, 2015). Particles were automatically identified using RELION implemented Laplacian-of-Gaussian blob detection. Particles were extracted and 2D classified using RELION 4.1 (Scheres, 2012). At this stage processing was stopped. Generated 2D classes allowed for unambiguous mapping of the epitope for Ab Y to the N-domain of CALRmut. Data collection and processing details are shown in Table 10. A schematic illustration of the cryo-EM image was made and is shown in FIG. 6, which shows that Fab Y binds to the N-domain of CALRmut and Fab X binds to the C-terminal of CALRmut.

TABLE 10
Data collection and processing details as used in Cryo-EM.

| | |
|---|---|
| Magnification | 150,000 |
| Voltage (kV) | 200 |
| Electron exposure (e⁻/Å²) | 60 |
| Pixel size (Å) | 0.967 |
| Micrographs number | 1259 |
| Initial Particles number | 213759 |
| Particles number in analyzed 2D | 143878 |
| classes | |

### Example 10

CALR×CD3 bispecific antibodies were characterized for binding to TF1 cells expressing human CALRmut del52 or CALRmut ins5 in a dose-dependent manner, using FACS.

BsAb 18, a mock control IgG1 antibody (TT×CD3) with the same CD3 arm as BsAb 18, and negative control IgG1 antibody were formulated in PBS. Vibecotamab was purchased from Thermo Fisher Scientific (cat #PIMA542248; Eagleville, PA) and diluted in PBS.

TF1 parental cells were purchased from DSMZ (cat #ACC 334; Braunschweig, Germany). TF1 cells lines with stable expression of human CALRmut del52 and human CALRmut ins5 were generated with lentivirus using polybrene transfection reagent at a concentration of 10 μg/ml. Single cell clones for CALRmut del52 and CALRmut ins5, were selected after serial dilution of pooled cells and sorted using a cell sorter. Cells were cultured in RPMI 1640 (11875 093; Gibco, Waltham, MA) culture medium containing 10% heat inactivated FBS (cat #16140 071; Gibco) and 0.5 μg/mL of puromycin (cat #10131027; Gibco) at 37° C. with 5% CO₂. TF1 parental cells were maintained in RPMI culture medium containing 10% heat inactivated FBS with 2 ng/ml GM CSF (cat #300 03; Gibco), and isogenic TF1 CALRmut del52 and ins5 cells were maintained in RPMI culture medium containing 10% heat inactivated FBS with 2 ng/ml GM CSF and 0.5 ug/ml puromycin.

For the binding assay, cells were first stained with FcR block (cat #422302 or 101320; Biolegend) for 10 minutes at RT, followed by serial dilutions of antibodies for 45 minutes at 4° C. After incubation, cells were washed twice with staining buffer (cat #554657; BD Biosciences) followed by an incubation with PE labeled secondary antibody (cat #H10104; Life Technologies, Carlsbad, CA) and viability dye (cat #423114; Biolegend) for another 30 minutes in the dark at 4° C. Cells were then washed twice with stain buffer and centrifuged at 1200 rpm for 5 minutes at RT. Cell acquisition was performed using FACSymphony A3 (BD Biosciences) using FACSDIVA software (BD Biosciences). Data analysis was performed using FlowJo software (BD Biosciences). Dead cells were excluded during analysis. GraphPad Prism v9 (Dotmatics, Boston, Massachusetts) was used to plot the graphs.

The TF1 cell lines that were engineered to express human CALRmut del52 and ins5 were stained with BsAb 18 and other control antibodies at various concentrations. Binding of antibodies to the cells was measured by adding a fluorescent secondary detection antibody, followed by analysis with flow cytometry. Negative control IgG1 (RSV), mock control IgG1 antibody (TT×CD3), and vibecotamab antibodies were used as the negative control for this assay.

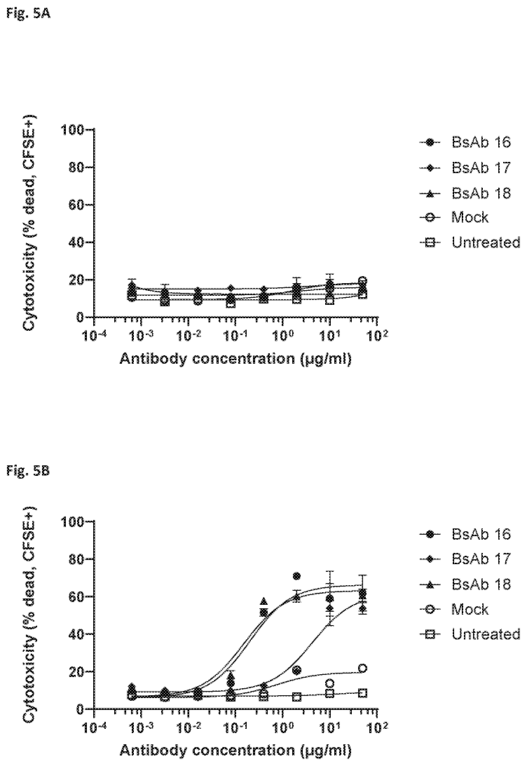

Results are shown in FIG. 7. Negative control IgG1 antibody (RSV) as well as mock control IgG1 antibody (TT×CD3) did not show any binding to the different TF1 cell types as these cells do not express their target antigens (i.e., RSV and human CD3, respectively). Vibecotamab showed a dose-dependent binding to all three TF1 cell types but the mean fluorescent intensity of binding was very low. BsAb 18 showed a dose-dependent increase in binding to both TF1 cells expressing CALRmut del52 and ins5 (FIGS. 7B and 7C, respectively) whereas it did not show any binding to TF1 parental cells, in the absence of its target antigen (FIG. 7A). Binding of BsAb 18 was evaluated in at least two different assays with an average EC50 value of 0.195±0.068 μg/mL for TF1 del52 cells and 0.083±0.051 μg/mL for TF1 ins5 cells.

### Example 11

CALR×CD3 bispecific antibodies were characterized for binding to Ba/F3 cells expressing CALRmut del52 and CALRmut ins5, in a dose-dependent manner, using FACS.

Ba/F3 cells were purchased from the DSMZ (cat #ACC 300). Ba/F3 cells lines with stable expression of MPL+CALRmut del52 or CALRmut ins5, were generated by nucleofection (Amaxa Cell Line Nucleofection Kit V and Lonza Nucleofector 4D Machine, Lonza, Basel, Switzerland). The cells were cultured in RPMI medium containing 10% heat inactivated FBS and 10 μg/mL of blasticidin (A11139-03; Fisher Thermo Scientific) for selection of CALR-positive cells. Single cell clones for CALRmut del52 and CALRmut ins5 were selected after serial dilution of pooled cells and sorted using cell sorter. The cells were cultured in RPMI medium containing 10% heat inactivated FBS at 37° C. with 5% CO₂. Ba/F3 parental cells were maintained in RPMI culture medium containing 10% heat inactivated FBS and 5 ng/mL of interleukin 3 (403-ML-025; R&D Systems) and isogenic del52 and ins5 cells were maintained in RPMI medium containing 10% heat inactivated FBS, 10 μg/mL of blasticidin and 2 μg/mL of puromycin.

Ba/F3 CALRmut del52 and ins5 cell lines that were engineered to express human CALRmut were stained with BsAb 18, mock control IgG1 antibody (TT×CD3) with the same CD3 arm as BsAb 18, negative control IgG1 antibody (RSV) and vibecotamab, at various concentrations. Binding of antibodies to the cells was measured by adding a fluorescent secondary detection antibody, followed by analysis with flow cytometry as described in Example 10. Negative control IgG1 antibody (RSV), mock control IgG1 antibody (TT×CD3) and vibecotamab antibodies were used as the negative control for this assay. Mock control IgG1 antibody (TT×CD3) has the same CD3 arm as BsAb 18.

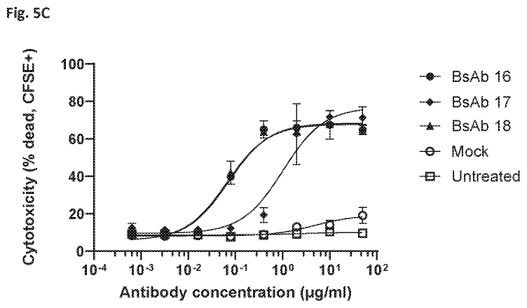

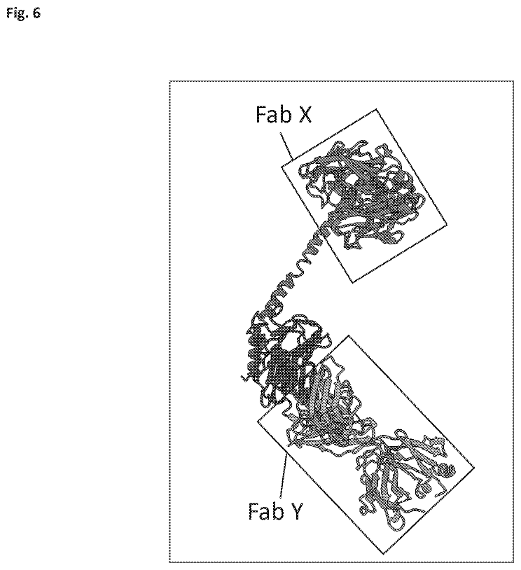

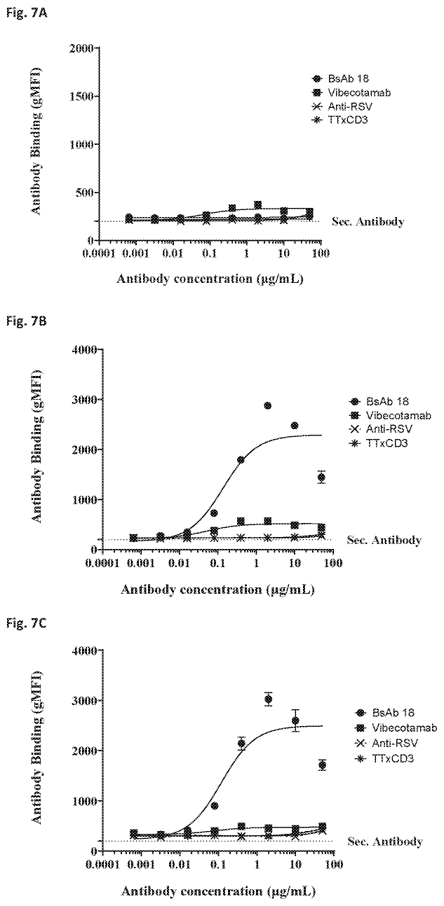

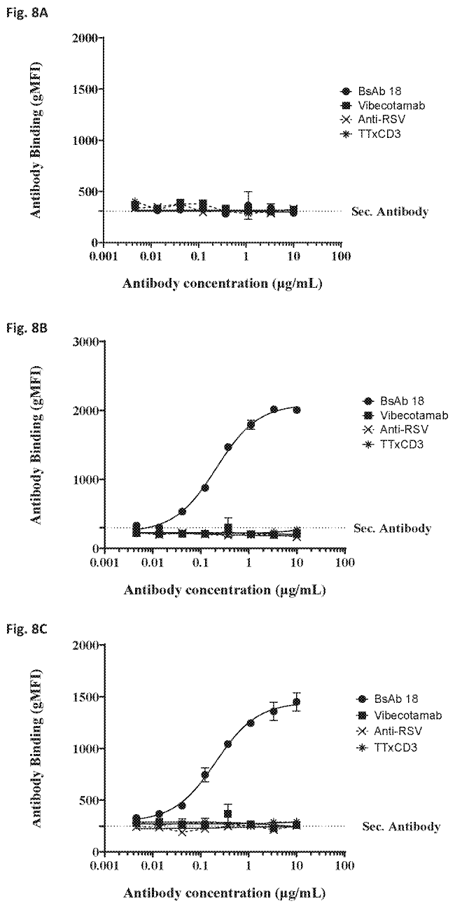

Results are shown in FIG. 8. Negative control IgG1 antibody (RSV), mock control IgG1 antibody (TT×CD3) as well as vibecotamab did not show any binding to any BaF3 cell types as these are murine cell lines and they do not express the target antigens of these antibodies. BsAb 18 showed a dose-dependent increase in binding to both Ba/F3 cells expressing CALRmut del52 and ins5 (FIGS. 8B and 8C) whereas it did not show any binding to Ba/F3 parental cells (FIG. 8A). Binding of BsAb 18 was evaluated in at least two different assays with an average EC50 value of 0.23±0.028 μg/mL for Ba/F3 CALRmut del52 cells and 0.259±0.073 μg/mL for Ba/F3 CALRmut ins5 cells.

### Example 12

CALR×CD3 bispecific antibodies were characterized for binding to primary CD34⁺ cells derived from a myelofibrosis (MF) patient donor sample, using FACS.

Deidentified peripheral blood sample from a patient with MF was obtained from Sanguine Biosciences. The donor presented CALRmut ins5 driver mutation with 89.6% variant allele frequency. PBMCs from whole blood were isolated by Ficoll gradient extraction according to the standard procedure (45-001-749; Thermo Fisher Scientific). CD34⁺ hematopoietic stem and progenitor cells were isolated from PBMCs using magnetic enrichment (130 100 453; Miltenyi Biotec).

Cord blood CD34⁺ cells from a healthy donor were obtained from StemCell Technologies (cat #70008.5). The CD34⁺ cells were cultured in complete serum-free expansion medium II media (cat #9655; StemCell Technologies) supplemented with IX P/S/G (cat #10378016; Life Technologies), 100 ng/mL of stem cell factor (cat #130-096-695; Miltenyi Biotec), 100 ng/mL of FLT3-L (cat #130-096-479; Miltenyi Biotec), 50 ng/mL of thrombopoietin (cat #130-095-752; Miltenyi Biotec), 10 μg/mL of low density lipoprotein (cat #2698; StemCell Technologies), 500 nM of StemRegenin 1 (cat #72342; StemCell Technologies), 35 nM of pyrimido-indole derivative (cat #1448724 09 1; Xcess Biotechnology).

CD34⁺ cells from the healthy and MF donors were stained with either BsAb 18 or mock control IgG1 antibody (TT×CD3) at various concentrations. Binding of antibodies to the cells was measured by adding a fluorescent secondary detection antibody, followed by analysis with flow cytometry as described in Example 10. Mock control IgG1 antibody (TT×CD3), which has the same CD3 binding domain as BsAb 18, was used as the negative control for this assay.

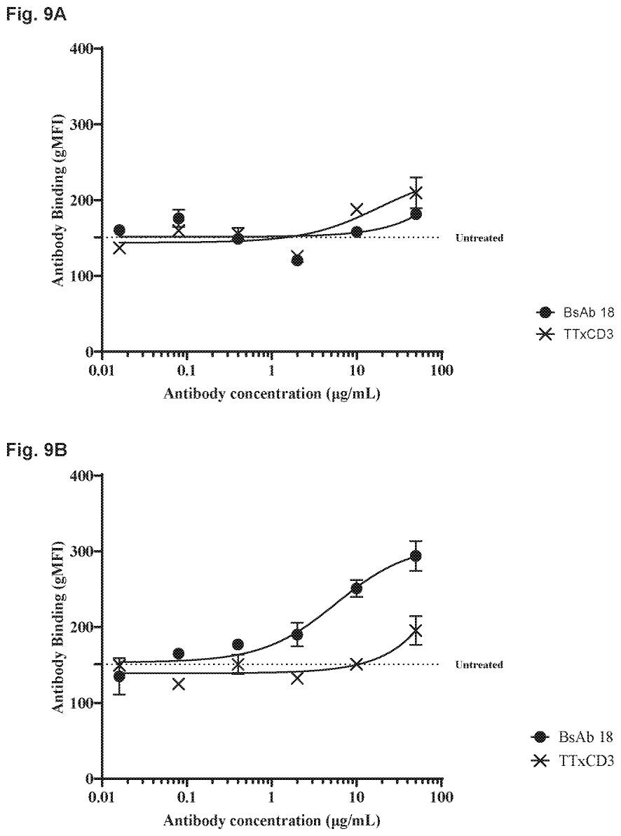

Results are shown in FIG. 9. Mock control IgG1 antibody (TT×CD3) did not show any binding to CD34⁺ cells from either the MF donor or the healthy donor. BsAb 18 showed a dose-dependent increase in binding to CD34⁺ cells from the MF donor (FIG. 9B) whereas it did not show any binding to CD34⁺ cells from the healthy donor (FIG. 9A). Binding of BsAb 18 was evaluated in at least two different assays with an average EC50 value of 5.775 μg/mL and an EC90 value of 51.975 μg/mL for MF donor CD34⁺ cells.

### Example 13

CALR×CD3 bispecific antibodies were characterized for binding to Reh cells with induced surface expression of wildtype calreticulin (CALRwt).

Given the N-domain specificity for the CALRmut arm of the binding moieties of the present disclosure, potential binding of BsAb 18 to the surface-translocated CALRwt on Reh cells was tested. Doxorubicin hydrochloride was purchased from Thermo Fisher Scientific® (Waltham, MA; cat #J64000.MA) and reconstituted in water. Reh cell line (ATCC number: CRL 8286™) was purchased from ATCC® (Manassas, VA). Reh cells were treated with doxorubicin 100 μM for 4 hours to induce surface translocation of CALRwt, as shown previously (Fucikova J. et al., Cancer Res 2011; 71:4821-4833).

Presence of ectopic CALRwt was confirmed by staining doxorubicin treated cells with a commercially available, fluorescent labeled CALRwt-detection antibody (Abcam; cat #ab209577) and its isotype control (Abcam; cat #ab209478) using standard surface cell staining (data not shown). The signal was detected by flow cytometry using a BD FACSymphony™ A3 cell analyzer (BD Biosciences) using BD FACSDiva™ v8.5 software (BD Biosciences). Dead cells were excluded from the analysis.

Binding of BsAb 18 along with control antibodies to CALRwt exposed on the surface of Reh cells was then tested. The control antibodies tested were Ab Y which is a bivalent monospecific antibody comprising two heavy and light chain variable regions having an amino acid sequence set forth in SEQ ID NO: 8 and SEQ ID NO: 23 respectively, Ab Z, which is known to bind CALRwt and an isotype control antibody. All antibodies were incubated with the cells at 50 μg/mL concentration, followed by detection with a fluorescent labeled secondary antibody.

To test for binding, cells were incubated with TruStain FcX™ FcR block reagent (BioLegend; cat #422302, 101320) for 10 minutes at room temperature, followed by incubation with serially diluted antibodies for 45 minutes at 4° C. After incubation, the cells were washed twice with staining buffer (BD Biosciences, Franklin Lakes, NJ; cat #554657) followed by an incubation with Invitrogen PE-labeled secondary and Zombie Violet viability dye for another 30 minutes in the dark at 4° C. The cells were then washed twice with staining buffer and centrifuged at 1200 rpm for 5 minutes at room temperature. Cell acquisition was performed using a BD FACSymphony A3 cell analyzer using BD FACSDiva v8.5 software. Data analysis was performed using FlowJo™ v10.8.1 software (BD Biosciences). Dead cells were excluded during analysis. GraphPad Prism 9 software (GraphPad Software, Boston, MA) was used to plot the graphs.

Result for dose-response curves of antibody binding are shown in FIG. 10. BsAb 18 antibody did not show any binding activity for CALRwt at the various doses tested. Binding was also assessed using Ab Y, which is a bivalent monospecific antibody with two CALR binding domains as in BsAb 18. Even with the higher avidity of Ab Y for the same CALR epitope that BsAb 18 recognizes, there was no binding observed for CALRwt on doxorubicin-treated Reh cells. However, Ab Z, an antibody that targets CALRwt, showed binding to surface CALRwt on Reh cells at high concentrations.

BsAb 18 therefore does not bind to CALRwt translocated to the cell surface in response to doxorubicin, a known stress inducer that causes surface translocation of CALRwt. It is hypothesized that this may be due to masking of the N-domain of CALRwt upon association with another cell surface protein upon induced translocation to the cell surface. It is further hypothesized that BsAb 18 recognizes the N-domain of CALR when it is exposed on the cell surface as CALRmut ins5 and CALRmut del52 and/or when CALR forms a complex on the cell surface with TPO-R.

### Example 14

CALR×CD3 bispecific antibodies were characterized for binding to CALRmut expressing target cells in the presence of patient plasma in the presence of soluble CALR to investigate if the binding of BsAb 18 to surface expressed CALRmut is affected in the presence of soluble CALR.

Effects on binding of BsAb 18 or mock control IgG1 antibody (TT×CD3) to TF-1 cells expressing CALRmut ins5 in the presence of patient plasma or healthy plasma was assessed (plasma donors from Sanguine Biosciences, Woburn, MA). Blood was delivered in sodium heparin (Na Hep) treated tubes and mixed. Tubes were spun at 1500×g for 10 minutes at room temperature and plasma was isolated, aliquoted, and stored at −80° C. Multiple enzyme-linked immunosorbent assays (ELISAs) were performed to detect soluble mutant CALR in the isolated plasma.

CALRmut ins5 patient and healthy donor plasma was obtained and thawed on ice. Antibodies were prepared at 10× working concentration (1000 μg/mL for a final starting concentration of 100 μg/mL) in a 2 mL 96-well assay plate (Corning #3960; Corning, NY) in PBS. Serial dilutions were prepared by adding 120 μL in 480 μL of PBS for a 5× dilution across a 7-point dose curve for all antibodies. 20 μL diluted antibodies was added in duplicate to a new 96-well assay plate. After thawing, 80 μL of plasma (neat or diluted in PBS) was added to the antibodies at 2× the working concentration (final working concentrations: 40%, 20%, 10%, 5%, 0%). 100 μL of antibody and plasma (both 2× final concentration) were pre-incubated at 4° C. for 45 minutes in the dark. After preparation, TF-1 CALRmut ins5 cells (prepared as in Example 10) were resuspended in BSA stain buffer (BD Biosciences: #554657) at a final concentration of ~0.79 million cells/mL. 5 μL per well of Fc block (#422302; Biolegend; San Diego, CA) was added and cells incubated for 10 minutes at 4° C. (final concentration of cells ~0.75 million cells/mL). A total of 100 μL of cells was added to the plates containing antibody and plasma after the 45-minute incubation without washing (after addition, antibody and plasma are at 1×; each well contained 75,000 cells). Cells, antibody, and plasma were incubated for 45 minutes at 4° C. in the dark. Plates were washed with 500 μL PBS and centrifuged at 1200 rpm for 5 minutes. Supernatant was discarded and plates washed again with 500 μL PBS and centrifuged again at 1200 rpm for 5 minutes. After the second wash, all wells were resuspended with 100 μL of BSA stain buffer containing secondary antibody (F(ab′)2-Goat anti-Human IgG Fc Secondary Antibody; #H10104; Invitrogen; 1:100 dilution) and Zombie NIR (Live/Dead marker, #423106; Biolegend; 1:100 dilution) and incubated for 45 minutes at 4° C. in the dark. After incubation, cells were washed with 500 μL BSA stain buffer and centrifuged for 5 minutes at 1200 rpm. Supernatant was discarded and 125 μL of BSA stain buffer added prior to data acquisition on a FACS machine (BD Symphony A5). Controls included untreated cells in the presence of all levels of plasma and cells stained with secondary antibody only. Data analysis was performed using FlowJo software (v10.6.2), and GraphPad Prism (v10.2.3) was used to plot PE anti-IgG Fc secondary gMFI.

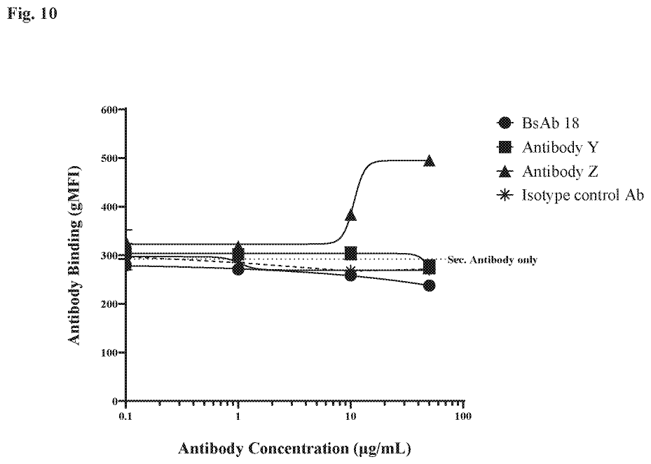

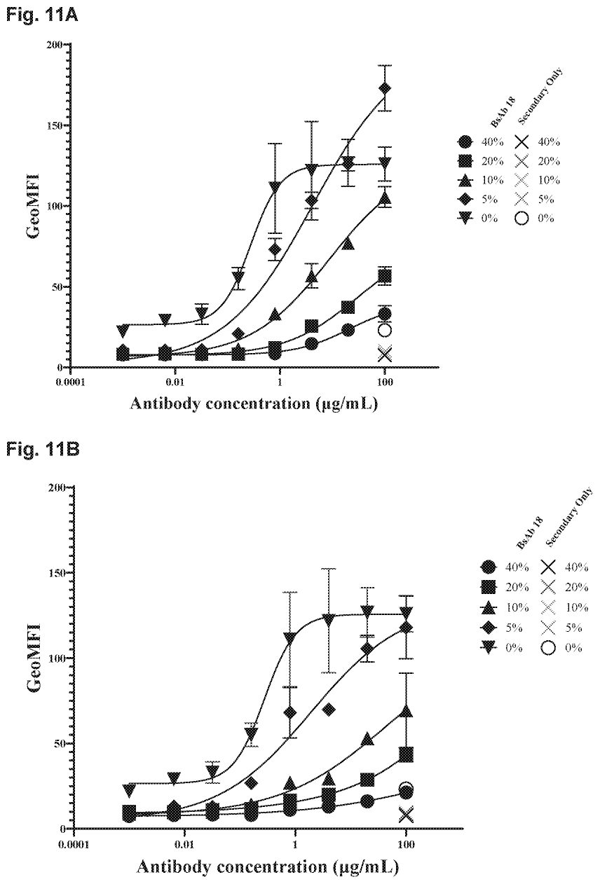

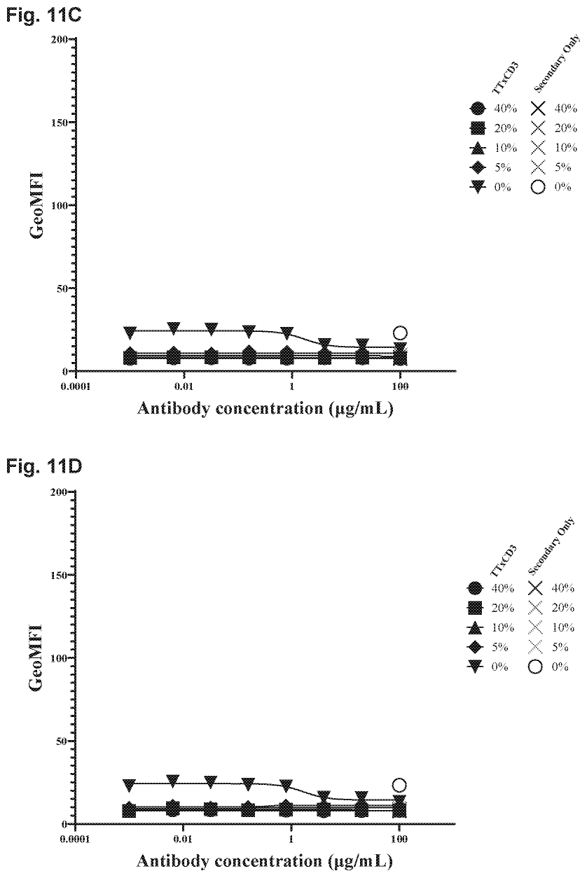

Comparison of binding of BsAb 18 and mock control IgG1 antibody (TT×CD3) to TF-1 CALRmut ins5 cells after pre-incubation with various concentrations of patient or healthy donor plasma revealed a plasma concentration dependent response for BsAb 18 (FIGS. 11A and 11B) whereas mock control IgG1 antibody (TT×CD3) showed no binding across all plasma concentration (FIGS. 11C and 11D). In other words, no significant difference was observed between the binding profile of BsAb 18 after pre-treatment with patient plasma or with healthy plasma for all concentrations of plasma. Secondary antibody alone showed no binding for either BsAb 18 or mock control IgG1 antibody (TT×CD3) across all conditions.

To determine if the difference in binding profiles could be fully accounted for by increasing concentrations of plasma, the fold change in binding for the top four concentrations compared to the untreated control values (denoted as % of Untreated Control: No Plasma) was investigated. The top four concentrations were chosen to represent the saturation point for maximal binding of BsAb 18 to TF-1 CALRmut ins5 cells without the presence of plasma. Analysis of BsAb 18 revealed that for all four concentrations, a 2-fold increase in binding for both the patient and healthy donor plasma conditions was consistent with a 2-fold decrease in the plasma concentration (data not shown). No significant difference was found between the binding profile of BsAb 18 after pre-treatment with patient or healthy plasma for all concentrations consistent with the gMFI data. Mock control IgG1 antibody (TT×CD3) had no significant increase in binding, consistent with the inability of this antibody to bind TF-1 CALRmut ins5 cells (data not shown).

These results show that BsAb 18 had a dose-dependent binding to TF-1 CALRmut ins5 cells in the presence of patient plasma containing soluble CALRmut ins5 and healthy donor plasma. Increasing volumes of plasma influenced binding potential; however, this was found to be independent of patient or healthy plasma. Analyses of the top four concentrations of BsAb 18, which represent the saturation point of binding, showed the inhibition of binding was directly correlated with increasing concentration of plasma in the system for both patient and healthy plasma.

### Example 15

It was investigated if the presence of soluble CALRmut affects the binding of CALR×CD3 bispecific antibodies to TF-1 cells expressing CALRmut del52 and CALRmut ins5.

In Example 14, exact concentrations of soluble CALRmut in the patient plasma could not be accurately assessed due to variability from the ELISA methods. To control for the soluble CALR fraction, a known concentration of purified CALRmut protein del52 was used instead of patient or healthy donor plasma. The soluble CALRmut protein used in this binding assay was MBP-fused (MBP-CALRmut with no cleavage site) del52 (Benchling ID: A0000380002, 1.4 mg/mL). The experiment was conducted by either pre-incubating the antibodies with soluble CALRmut or without pre-incubation before exposure to target cells. Antibodies tested were mock control IgG1 antibody (TT×CD3) and BsAb 18, and binding was assessed on TF-1 CALRmut del52 cells or TF-1 CALRmut ins5 cells as described in Example 10.

In a 96-well assay plate (Corning #3960; Corning, NY), 2-fold serial dilutions of soluble CALRmut protein del52 were prepared at 4× the working concentration in PBS (final highest working concentration 200 ng/mL). In a separate 96-well assay plate, antibodies were prepared at 2× the working concentration (final highest working concentration 50 μg/mL). A 5-fold serial dilution across 8 points was prepared in PBS.

Soluble CALR Protein Binding Assay to TF1 CALRmut Del52 and Ins5 Cells without Pre-Incubation:

In a separate 96-well plate either TF-1 CALRmut del52 cells or TF-1 CALRmut ins5 cells as described in Example 10 were prepared at ~2.2 million cells/mL in BSA stain buffer (BD Biosciences: #554657). 5 μL per well of Fc block (#422302; Biolegend; San Diego, CA) was added and cells incubated for 10 minutes at 4° C. (final concentration of cells ~2 million cells/mL). 50 μL of cells (100K cells per well) was added to each well and 50 μL of serially diluted soluble CALRmut protein del52 was added to designated well and allowed to incubate for 45 minutes at 4° C. Without washing, 100 μL of diluted antibodies was added into designated wells and allowed to incubate for 45 minutes at 4° C. in the dark.

Soluble CALR Protein Binding Assay to TF1 CALRmut Del52 Cells with Pre-Incubation:

100 μL of antibodies and 50 μL soluble CALRmut protein del52 were pre-incubated together for 45 minutes at 4° C. In a separate 96-well plate TF-1 CALRmut del52 cells as described in Example 10, were prepared at ~2.2 million cells/mL in BSA stain buffer (BD Biosciences: #554657). 5 μL per well of Fc block (#422302; Biolegend; San Diego, CA) was added and cells incubated for 10 minutes at 4° C. (final concentration of cells ~2 million cells/mL). 50 μL of cells (100K cells per well) was added to each designated well of pre-incubated antibody and soluble CALRmut. Wells were incubated for 45 minutes at 4° C. in the dark.

Plates from both experiments were then centrifuged at 1200 rpm for 5 minutes. Wells were washed with 20 μL of PBS and centrifuged again at 1200 rpm for 5 minutes. After wash, all wells were resuspended with 100 μL of BSA stain buffer containing secondary antibody (F(ab′)2-Goat anti-Human IgG Fc Secondary Antibody; #H10104; Invitrogen; 1:100 dilution) and Zombie Violet (Live/Dead marker, #423114; Biolegend; 1:100 dilution) and incubated for 45 minutes at 4° C. in the dark. After incubation, cells were washed with 1000 μL BSA stain buffer and centrifuged for 5 minutes at 1200 rpm. Supernatant was discarded and 75 μL of BSA stain buffer added prior to data acquisition on a FACS machine (BD Symphony A3). Controls included cells stained with secondary antibody only. Data analysis was performed using FlowJo software (v10.6.2), and GraphPad Prism (v10.2.3) was used to plot PE anti-IgG Fc secondary gMFI.

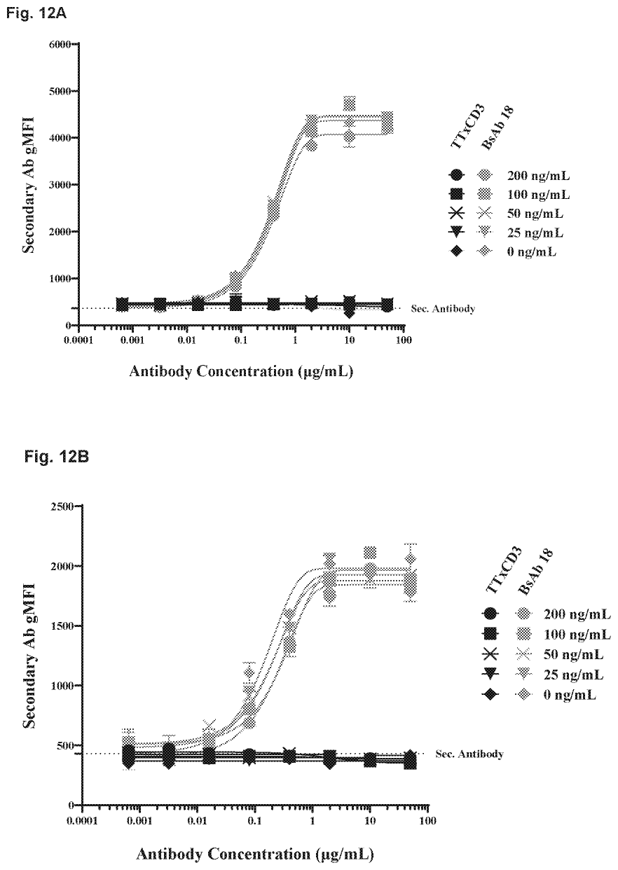

Results are shown for binding to TF-1 CALRmut del52 cells with no pre-incubation (FIG. 12A), and binding to TF-1 CALRmut ins5 cells with no pre-incubation (FIG. 12B). Binding to TF-1 CALRmut del52 cells with pre-incubation of soluble CALR was also tested as described above (data not shown). Results show that CALRmut del52 soluble protein had no impact on the binding of BsAb 18 to TF-1 CALRmut del52 cells or to TF-1 CALRmut ins5 cells with or without pre-incubation of soluble CALR and antibodies. No binding was observed for mock control IgG1 antibody TT×CD3 for either cell line (FIG. 12A, 12B) and was consistent with secondary antibody staining alone. These results are consistent with the results obtained with plasma in Example 14 and, taken together, demonstrate that soluble CALRmut does not interfere with the binding moieties of the present disclosure with respect to their binding to cell surface-expressed CALRmut.

### Example 16

CALR×CD3 bispecific antibodies were characterized in a T-cell cytotoxicity assay using isogenic TF1 cells expressing CALRmut del52 and CALRmut ins5.

TF1 parental cells and TF1 CALRmut del52 and ins5 cell lines that had been engineered to express human CALRmut, as described in Example 10, were used as target cells and co-cultured with T cells (effector cells) from healthy donors along with different antibodies, including BsAb 18, at various concentrations for 72 hours. Total T cells were isolated from fresh leukopak from healthy donors (StemCell Technologies, cat #70500; Vancouver, BC) using T cell isolation kits (StemCell Technologies, cat #17951) according to manufacturer's instructions. Cytotoxicity of target cells was analyzed with flow cytometry.

Target cells were labeled with CFSE (Life Technologies, cat #C3454; Carlsbad, CA) according to manufacturer's protocol. CFSE labeled target cells were then incubated with Fc block (Biolegend, cat #4422302, San Diego, CA) for 10 minutes at RT. Serial dilutions of antibody were added into designated wells followed by T cell addition at effector to target (E:T) ratio of 5:1. Co-cultures were incubated for 72 hours at 37° C. with 5% CO₂. After 72 hour incubation, plates were centrifuged for 5 minutes at 1200 rpm. Supernatant was removed for MSD analysis (Human Proinflammatory Panel kit K15049D-2; Meso Scale Discovery, Rockville, MD) and run according to the manufacturer's recommendations. For flow cytometry, cells were washed twice with stain buffer (BD Biosciences, cat #554657, Franklin Lakes, NJ) followed by staining with anti-CD69 (Biolegend, cat #310938) and viability dye (Biolegend, cat #423114) for 30 minutes in the dark at 4° C. Cells were then washed twice with stain buffer and centrifuged at 1200 rpm for 5 minutes at room temperature. Cell pellet was resuspended in 100 μl stain buffer. Cell acquisition was performed under FACSymphony A3 (BD Biosciences) using DIVA software (BD Biosciences). Data analysis was performed using FlowJo software (BD Biosciences). For percentage cytotoxicity, cells were gated as total cells/CFSE+ tumor cells/dead cells. For percentage T cell activation (see Example 17), cells were gated as total cells/CFSE negative T cells/live cells/CD69 positive cells. GraphPad Prism (v9, Dotmatics, Boston, MA) was used to plot the graphs.

Mock control IgG1 antibody (TT×CD3) and Ab X, which binds to the mutant C-terminus of CALR, were used as the negative controls and vibecotamab was used as positive control for cytotoxicity measurement. The mock control IgG1 antibody (TT×CD3) has the same CD3 binding domain as BsAb 18.

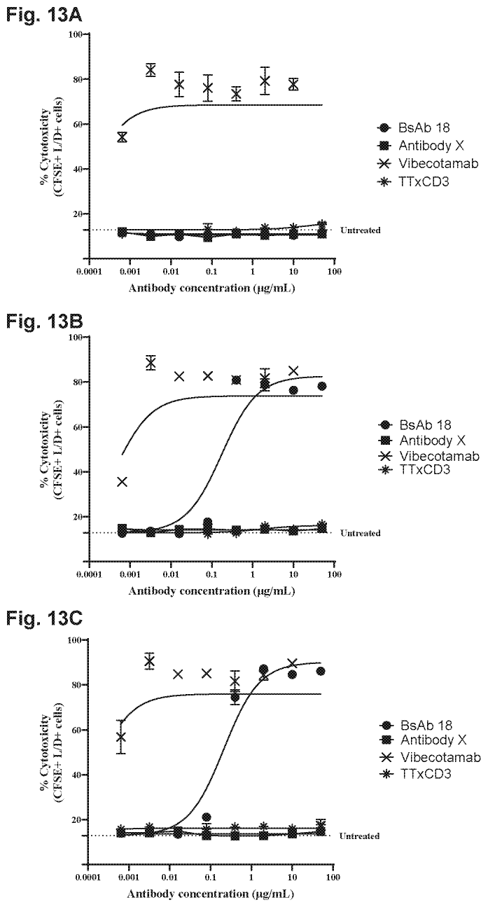

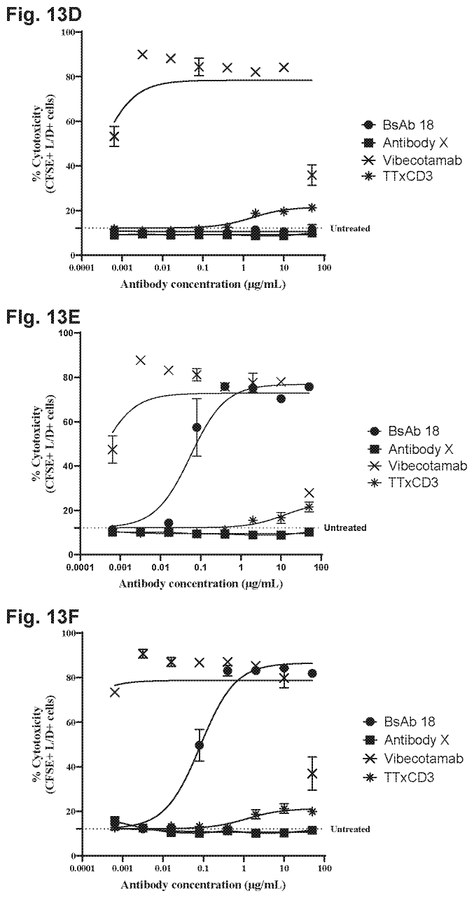

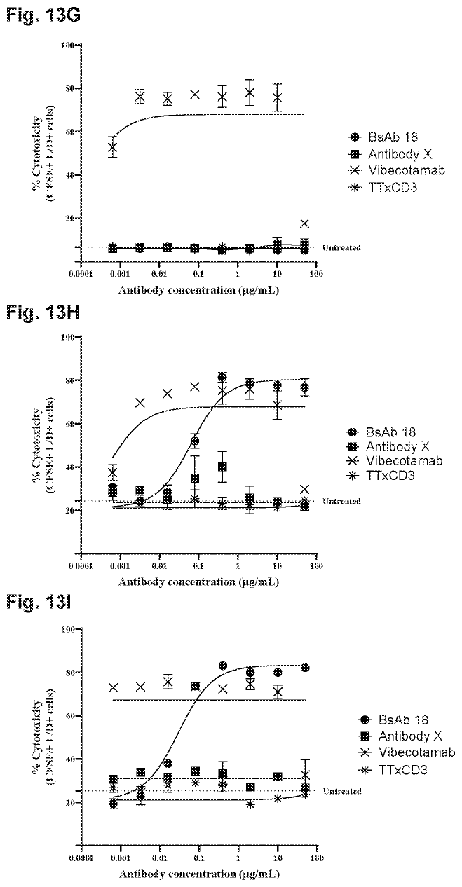

Results are shown in FIG. 13 for 4 donors. The mock control IgG1 antibody (TT×CD3) as well as Ab X did not show any killing of TF1 CALRmut expressing cells. Vibecotamab showed dose dependent killing of all 3 cell types. BsAb 18 showed dose dependent killing of both TF1 CALRmut del52 cells (FIG. 13B, E, H, J) and CALRmut ins5 cells (FIG. 13C, F, I) whereas it did not show any cytotoxicity in TF1 parental cells (FIG. 13A, D, G). BsAb 18 mediated TF1 CALRmut del52 and ins5 cytotoxicity was evaluated in at least three different assays with four T cell donors and the average EC50 value was calculated, 0.134±0.067 μg/mL for TF1 CALRmut del52 cells and 0.109±0.094 μg/mL for TF1 CALRmut ins5 cells. Average AUC was calculated to be 455±167 for TF1 parental cells, 3819 t 115 for TF1 CALRmut del52 cells and 4147±102 for TF1 CALRmut ins5 cells.

### Example 17

CALR×CD3 bispecific antibodies were characterized for target-mediated T cell activation in isogenic TF1 cells expressing CALRmut del52 and CALRmut ins5.

Isogenic TF1 cell lines that had been engineered to express human CALRmut were used as target cells and co-cultured with T cells (effector cells) from healthy donors along with different antibodies, including BsAb 18, at various concentrations for 72 hours and total T cell activation was analyzed with flow cytometry, as described in Example 16. For percentage T cell activation, cells were gated as total cells/CFSE negative T cells/live cells/CD69 positive cells. GraphPad Prism (v9, Dotmatics, Boston, MA) was used to plot the graphs.

The mock control IgG1 antibody (TT×CD3) and Ab X, which binds to the mutant C-terminus of CALR, were used as negative controls and vibecotamab was used as positive control for T cell activation measurements. The mock control IgG1 antibody (TT×CD3) has the same CD3 binding domain as BsAb 18.

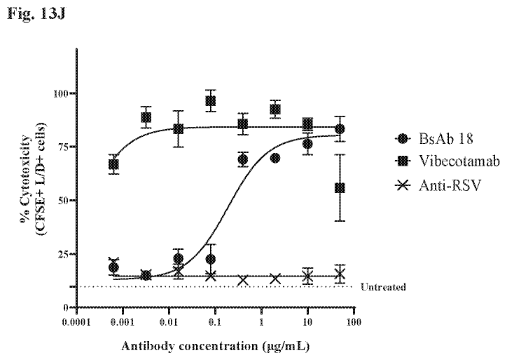

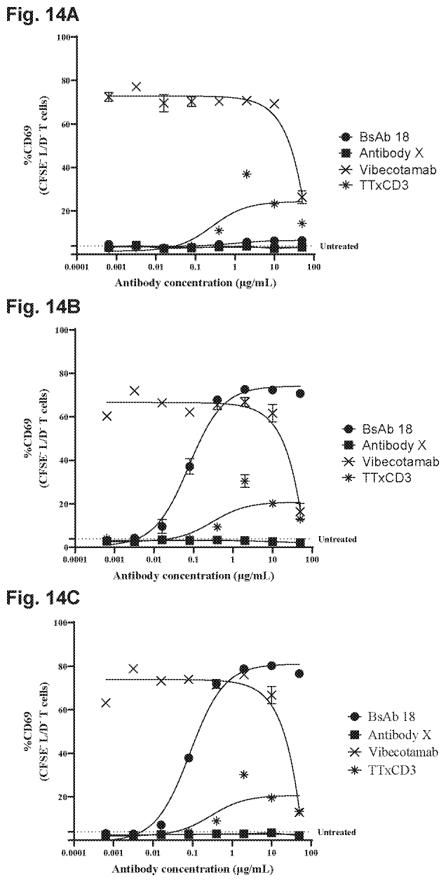

Results are shown in FIG. 14 for a representative donor. Ab X did not show any T cell activation in any of the co-cultures. Vibecotamab showed dose-dependent T cell activation in co-cultures with all three cell types. The mock control IgG1 antibody (TT×CD3) also showed T cell activation but potency of activation was very low. BsAb 18 showed dose dependent T cell activation in both TF1 CALRmut del52 and ins5 cell co-cultures (FIGS. 14B and 14C) whereas it did not show any activation of T cells in TF1 parental-T cell co-cultures (FIG. 14A). BsAb 18 mediated T cell activation was evaluated in at least three different assays, with multiple T cell donors and the average EC50 value was calculated, 0.047±0.025 μg/mL for TF1 CALRmut del52 cells and 0.050±0.033 μg/mL for TF1 CALRmut ins5 cells.

### Example 18

CALR×CD3 bispecific antibodies were characterized for target-mediated CD8+ T cell proliferation of isogenic TF1 cells expressing CALRmut del52 and ins5.

Isogenic TF1 cell lines that had been engineered to express human CALRmut, as described in Example 10, were used as target cells and co-cultured with CFSE-labeled T cells (effector cells) from healthy donors along with different antibodies, including BsAb 18, at various concentrations. Total T cells isolated from healthy PBMCs were used as effector cells. CD8+ T cell proliferation was analyzed with flow cytometry. T cells were labeled with CFSE (Life Technologies, cat #C3454) according to manufacturer's protocol.

TF1 target cells were first incubated with Fc block (Biolegend, cat #4422302) for 10 minutes at RT. Serial dilutions of antibody were added into designated wells followed by CFSE-labeled T cell addition at effector to target (E:T) ratio of 5:1. Co-cultures were incubated for 5 days at 37° C. with 5% CO₂. After 5-day incubation, cells were washed twice with stain buffer (BD Biosciences, cat #554657) followed by staining with T cell surface markers and viability dye (Biolegend, cat #423114) for 30 minutes in the dark at 4° C. Cells were then washed twice with stain buffer and centrifuged at 1200 rpm for 5 minutes at room temperature. Cell pellet was resuspended in 100 μl stain buffer. Cell acquisition was performed under FACSymphony A3 (BD Biosciences) using DIVA software (BD Biosciences). Data analysis was performed using FlowJo software (BD Biosciences). For percentage CD8+ T cell proliferation, cells were gated as total cells/Live cells/CD8+ T cells/Frequency of CFSE positive CD8 T cells. GraphPad Prism was used to plot the graphs.

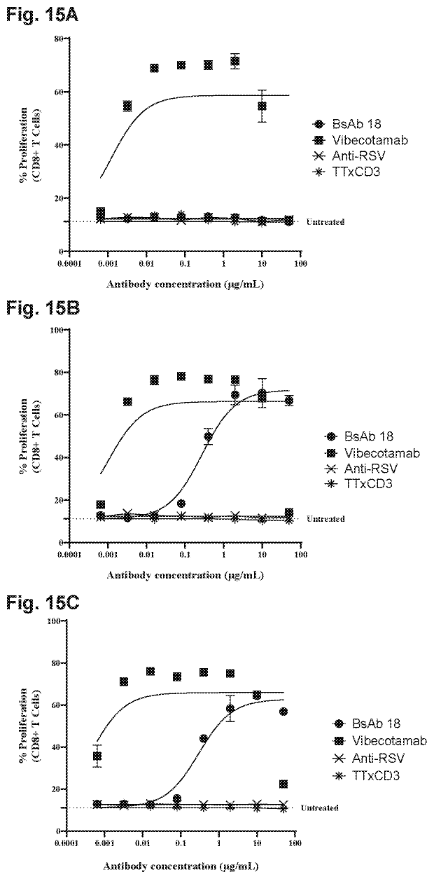

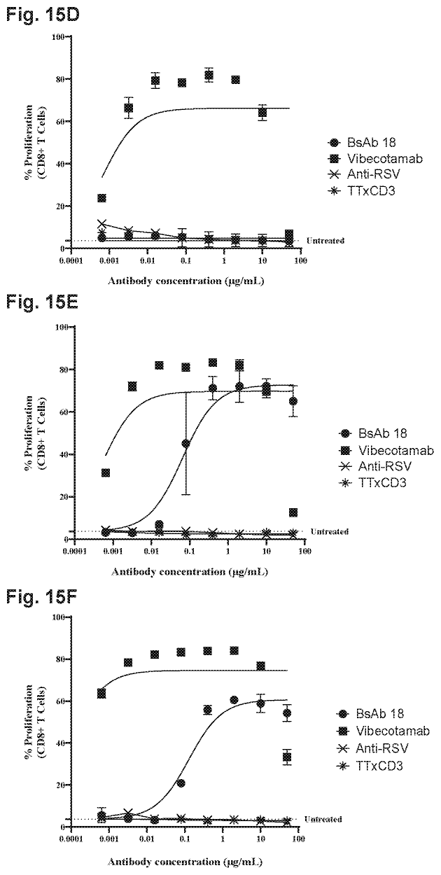

Results are shown in FIG. 15 for two different T cell donors (FIG. 15A-C represents donor 1 and FIG. 15D-F represents donor 2). The mock control IgG1 antibody (TT×CD3) and negative control IgG1 antibody (anti-RSV) antibody were used as the negative control while vibecotamab was used as positive control for T cell proliferation measurements. The mock control IgG1 antibody (TT×CD3) has the same CD3 binding domain as BsAb 18. The mock control IgG1 antibody (TT×CD3) as well as negative control IgG1 antibody (anti-RSV) did not show any CD8+ T cell proliferation in the co-cultures. Vibecotamab showed dose dependent CD8+ T cell proliferation in co-cultures with TF1 parental cells, TF1 CALRmut del52 and ins5 cells. BsAb 18 showed dose dependent CD8+ T cell proliferation in co-cultures with TF1 CALRmut del52 (FIG. 15B, 15E) and ins5 cells (FIG. 15C, 15F) whereas it did not show any proliferation of CD8+ T cells in co-cultures with TF1 parental cells (FIG. 15A, 15D). BsAb 18 mediated CD8+ T cell proliferation was evaluated in at least two different assays, with two different T cell donors and the average EC50 value was calculated, 0.171±0.150 μg/mL for TF1 CALRmut del52 cells and 0.213±0.119 μg/mL for TF1 CALRmut ins5 cells.

### Example 19

CALR×CD3 bispecific antibodies were characterized in a T-cell cytokine release assay in the presence of isogenic TF1 cells expressing CALRmut del52 and ins5.

Isogenic TF1 cell lines that had been engineered to express human CALRmut as described in Example 10 were used as target cells and co-cultured with T cells (effector cells) from healthy donors along with different antibodies, including BsAb 18, at various concentrations for 72 hours. Supernatant was collected and used to measure cytokine levels using the MSD protocol as follows.

Proinflammatory Panel 1 V-PLEX (cat #K15049D-4, Meso Scale Diagnostics, Rockville, MD) containing IFN-γ, IL-10, IL-2, IL-4, IL-6, IL-8, IL-10, IL-12p70, IL-13, and TNF-α kits were used to measure cytokines. Plates were run according to manufacturer's recommendations and were read on the MESO SECTOR S 600MM using the Methodical Mind with TeamLink, ProductLink, and Instrument Link connectivity Fall 2020 (1.0.38) software. All data was analyzed using the MSD Discovery Workbench (LSR_4_0_13) software.

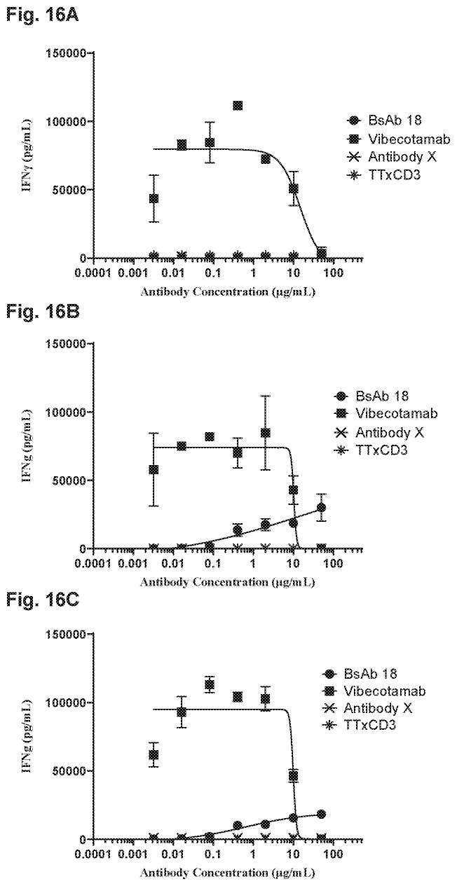

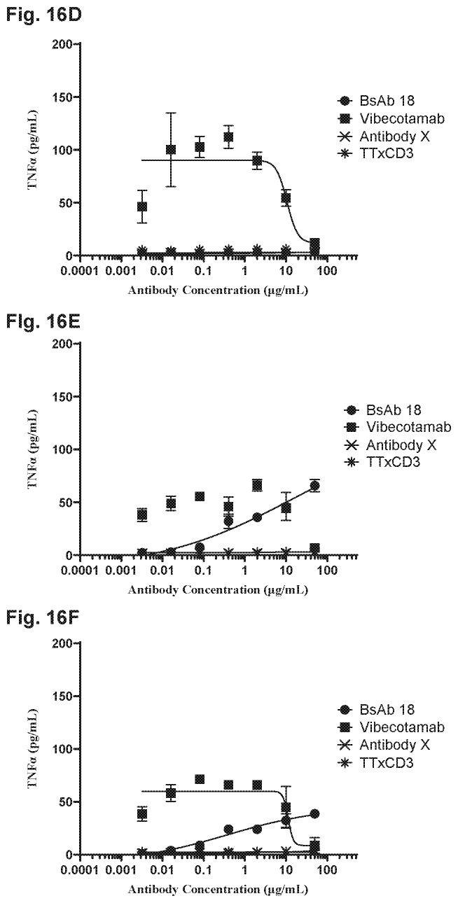

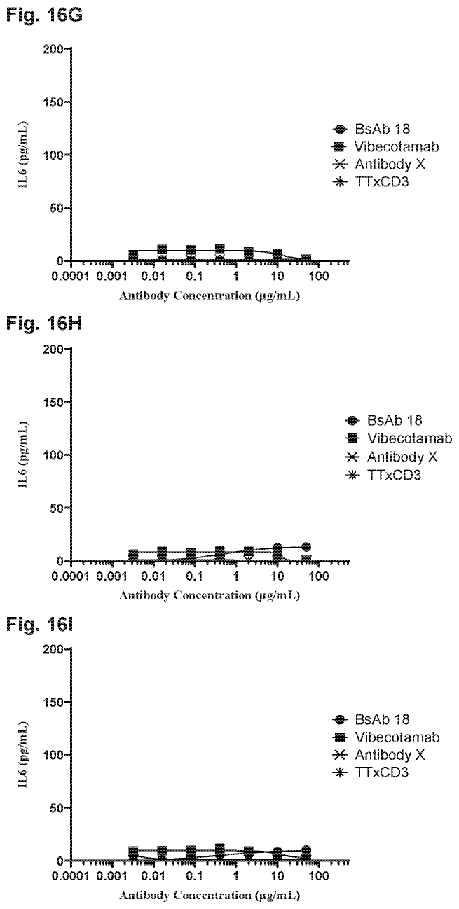

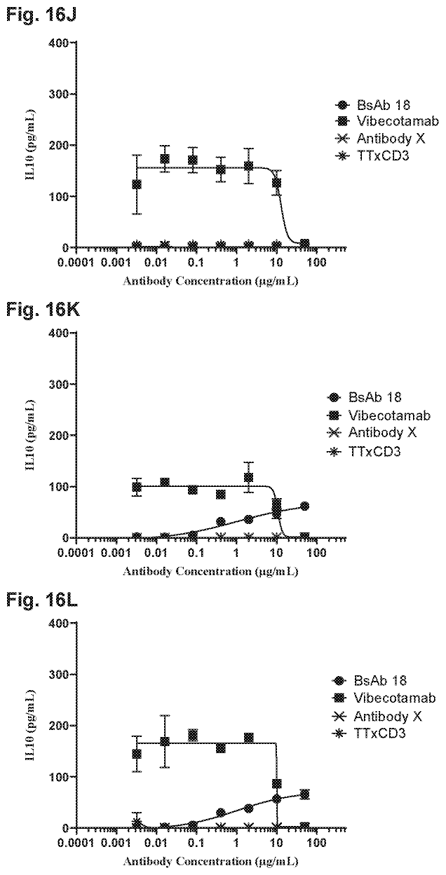

Results are shown in FIG. 16, which shows cytokine levels for IFN-γ (FIG. 16A-C), TNF-α (FIG. 16D-F), IL-6 (FIG. 16G-H), and IL-10 (FIG. 16J-L). For each cytokine, the three graphs represent cytokine levels from TF1 parental cells, TF1 cells expressing del52 and TF1 cells expressing CALRmut ins5. Further cytokines (IL-1b, IL-12p70, IL-2, IL-8) were tested but data is not shown.

The most prominent cytokine induced by BsAb 18 was IFN-γ in the presence of CALRmut-expressing TF1 cells (FIGS. 16B and C). BsAb 18 showed no induction of cytokines when incubated with target cells lacking CALRmut (parental TF1 cells as shown in FIGS. 16A, D, G and J) and showed low induction of cytokines when incubated with CALRmut expressing cells (between 5-100 μg/mL) for cytokines TNFα, IL2, IL6, IL10, and IL8, even at high concentrations of BsAb 18. There was no significant induction of cytokines at an antibody concentration of less than 0.1 μg/mL. In comparison, vibecotamab showed higher cytokine induction in all three cell lines. Ab X served as a negative control. Results are shown for one representative donor out of two donors tested. Thus, despite showing cytotoxicity against target cells expressing CALRmut, an analysis of cytokine induction in these assays revealed that only minimal levels of inflammatory cytokines are induced by BsAb 18.

### Example 20

CALR×CD3 bispecific antibodies were characterized for T cell activation and T cell-mediated cytotoxicity using MF patient-derived CD34+ cells as target cells.

The effect of BsAb 18 on CD34+ cells derived from MF patients with CALRmut del52 and ins5 mutations was assessed by co-culturing isolated CD34+ target cells with healthy donor T cells using techniques described in Examples 16 and 18. Binding of BsAb 18 to CD34+ cells isolated from two patients was confirmed and co-cultures were evaluated for the activation of CD8+ T cells in response to BsAb 18 and control antibodies as described in Example 18. The mock control IgG1 antibody (TT×CD3) and negative control IgG1 antibody (anti-RSV) antibody were used as the negative control antibodies.

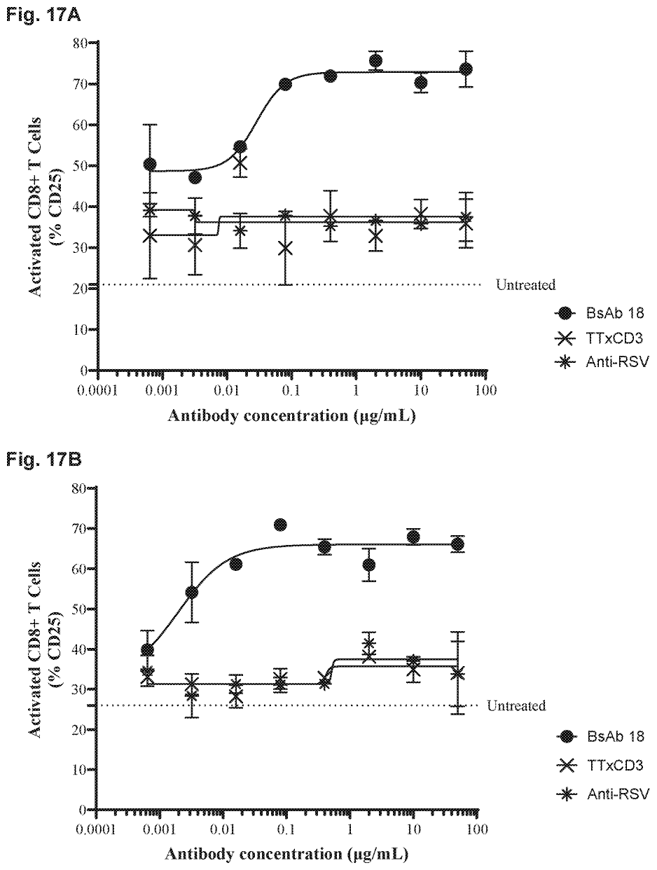

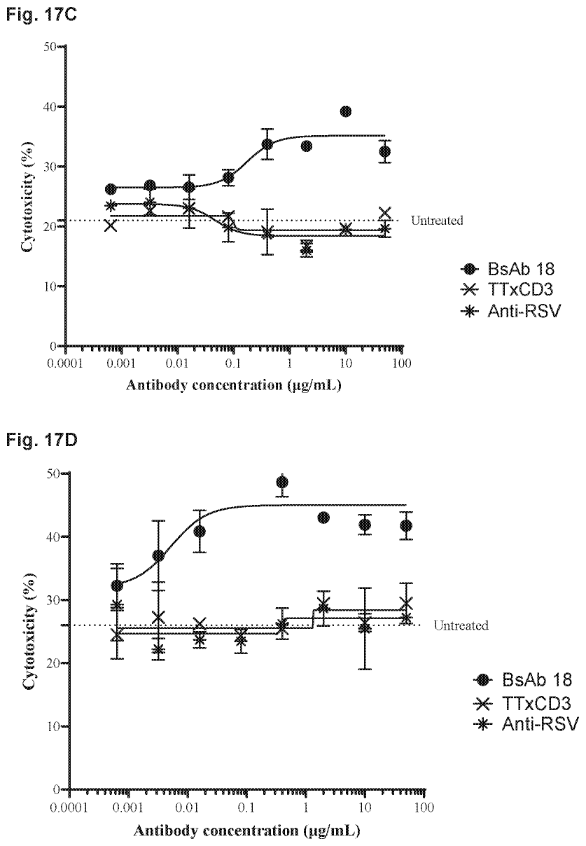

Results are shown in FIG. 17A-D. BsAb 18 induced a dose-dependent increase in activated CD25 expressing CD8+ T cells when tested together with MF patient derived CD34+ cells expressing CALRmut del52 (FIG. 17A) and CALRmut ins5 (FIG. 17B) whereas there was no increase in T cell activation observed with control antibodies. BsAb 18 also showed a dose-dependent increase in the cytotoxicity of target cells for both MF patient derived CD34+ cells expressing CALRmut del52 (FIG. 17C) and CALRmut ins5 (FIG. 17D, whereas no cytotoxicity was observed in response to control antibodies.

### Example 21

CALR×CD3 bispecific antibodies were characterized in a cytokine release assay, using T-cells from healthy donors or PBMCs from healthy donors. The ability of bispecific antibodies was evaluated to bind to CD4+ and CD8+ T cells, following which cytokine production potential as a result of engagement with healthy donor T cells was examined. Furthermore, cytokine induction potential of antibodies was tested by incubation with whole PBMCs from healthy donors.

T Cell Isolation and Phenotyping: Human leukopaks from healthy donors were obtained from STEMCELL Technologies or BioIVT. T cells were isolated using negative selection following the RoboSep™-C protocol (STEMCELL Technologies). Isolated T cells were phenotyped using the following cell surface markers: Zombie NIR Viability (#423106; Biolegend), human CD45 (Clone HI30; #304048; Biolegend), human CD19 (Clone HIB19; #982410; Biolegend), human CD14 (Clone HCD14; #325618; Biolegend), human CD3 (Clone UCHT1; #300434; Biolegend), human CD25 (Clone BC96; #302636; Biolegend), human CD69 (Clone FN50; #310938; Biolegend), human CD127 (Clone A019D5; #351316; Biolegend), human CD4 (Clone SK3; #612748, BD Biosciences), and human CD8 (Clone RPA-T8; #612943; BD Biosciences).

For cytokine analysis in T cells, T cells were thawed in AIMV media supplemented with AlbuMAX (#31035025; Gibco), spun at 400×g for 5 minutes, and resuspended in AIMV/AlbuMAX media at a final concentration of 1.11 million cells/mL. Fc block (#422302; Biolegend) was added and cells were incubated at room temperature for 5 minutes (final concentration of cells 1 million cells/mL). Cells were added into the 96-well assay plate (Corning #3879). Antibodies were prepared at 100 μg/mL (2× the antibody working concentration) in AimV/AlbuMAX media. Serial dilutions were prepared with 5× dilution for a 7 point dose curve for all antibodies. Controls included were vibecotamab (CD123×CD3: Invitrogen: MA5-42248), mock control IgG1 antibody (TT×CD3), negative control IgG1 anti-RSV antibody, and untreated T cells. Antibodies were added into the designated wells and the plate was incubated at 37° C. After 72 hours, plates were centrifuged and supernatant was removed for MSD analysis (Human Proinflammatory Panel kit #K15049D-2). For PBMCs, serial dilutions (starting at 50 μg/mL) were prepared with 5× dilution for a 7-point dose curve for all antibodies and were incubated with healthy donor PBMCs for 72 hours. Controls used included negative control IgG1 anti-RSV antibody, vibecotamab, mosunetuzumab and untreated cells. Supernatant was collected and used to measure cytokine levels using the MSD protocol. Two types of MSD kits were used to measure cytokines. First was the Proinflammatory Panel 1 V-PLEX (catalog #K15049D-4) containing IFN-γ, IL-1β, IL-2 IL-4, IL-6, IL-8, IL-10, IL-12p70, IL-13, and TNF-α. Plates were run according to manufacturer's recommendations and samples were diluted 1:2 in assay diluent. Plates were read on the MESO SECTOR S 600MM using the Methodical Mind™ with TeamLink™, ProductLink™, and Instrument Link™ connectivity Fall 2020 (v1.0.38) software. All data was analyzed using the MSD Discovery Workbench (LSR_4_0_13) software and GraphPad Prism (v10.2.3) was used to plot the data.

Antibodies (vibecotamab, mosunetuzumab, mock control IgG1 antibody (TT×CD3), and negative control IgG1 anti-RSV antibody, and BsAb 18) were first tested for binding to CD4+cells and CD8+ T cells T cells. BsAb 18 showed binding activity to CD4+ cells and CD8+ T cells, similar to the binding of vibecotamab to these cells (data not shown). Binding of BsAb 18 was dose-dependent and the average EC50 values for CD4+ and CD8+ cell binding were found to be 3.61 and 3.15 μg/mL, respectively.

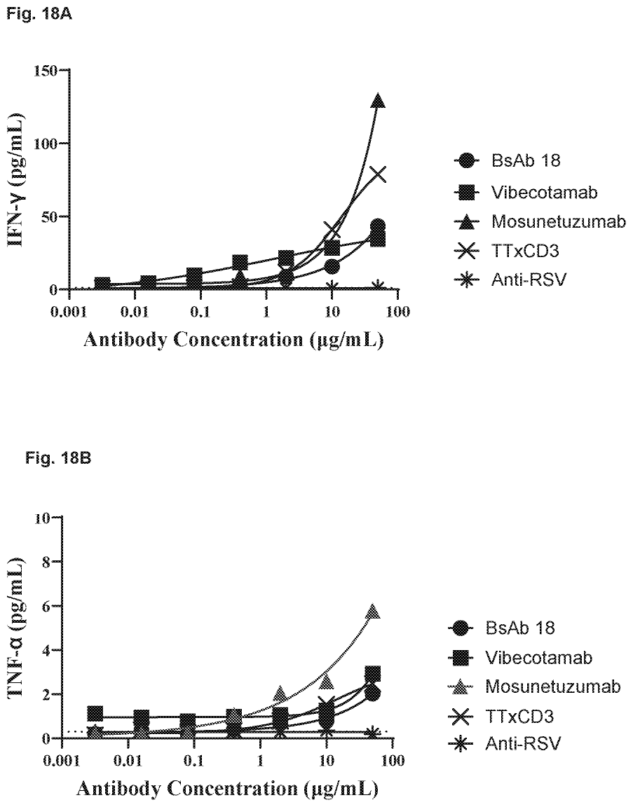

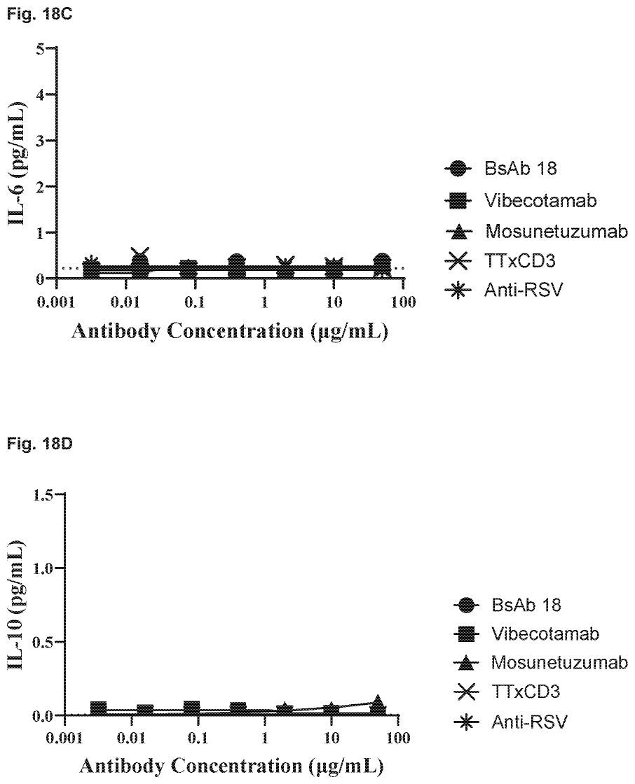

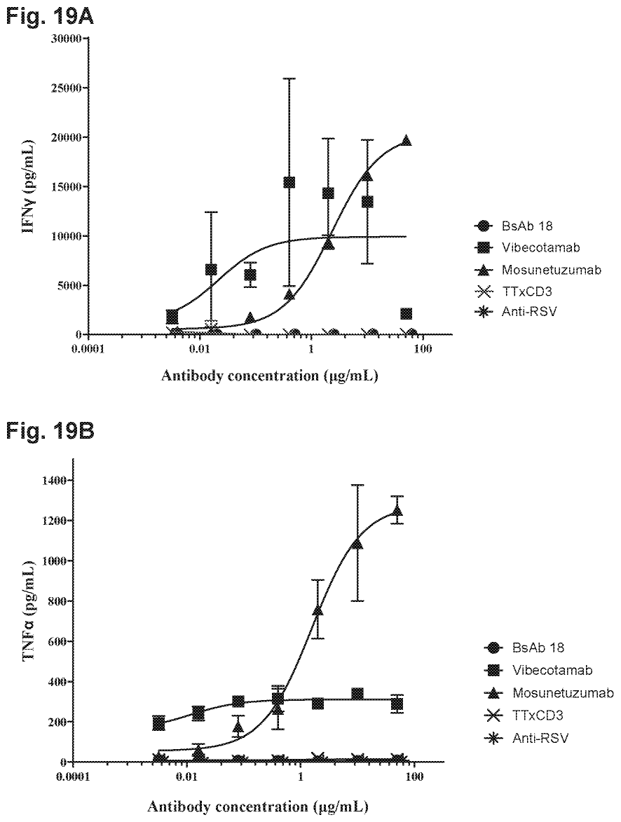

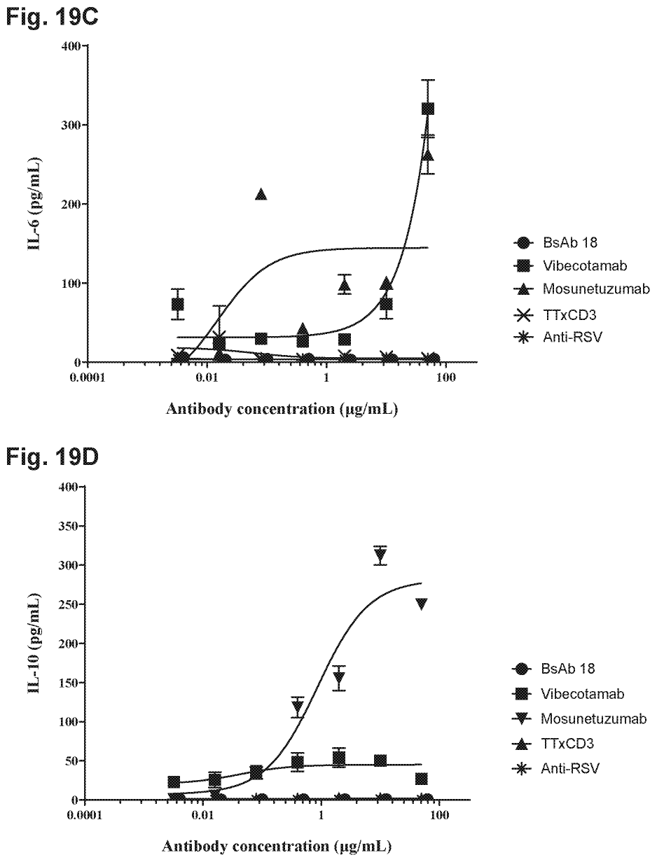

FIG. 18 shows cytokine analysis after incubation of antibodies with healthy donor T cells. FIG. 19 shows cytokine analysis after incubation of antibodies with healthy donor PBMCs. Results are shown for cytokines IFN-γ, TNF-α, IL-6, and IL-10 for T-cells and PBMCs from one representative donor. Further cytokines were tested, but data is not shown.

BsAb 18 showed binding activity to CD4+ cells and CD8+ T cells, similar to the binding of vibecotamab to these cells (data not shown). Despite binding to T cells, BsAb 18 did not induce high levels of CRS-associated cytokines in comparison to vibecotamab (FIG. 18A-D). The control antibodies vibecotamab and mosunetuzumab showed a dose-dependent induction of IFN-γ, TNF-α, and IL-10 across multiple donors.

In addition, BsAb 18 did not induce CRS-associated cytokines when incubated with healthy donor PBMCs (FIG. 19A-D). Control antibodies vibecotamab and mosunetuzumab, targeting CD123 and CD20 respectively, showed a dose-dependent induction of IFN-γ, TNF-α, and IL-10 across all donors. Together these results suggest that BsAb 18 effectively binds to T cells yet does not induce CRS associated cytokines.

### Example 22

CALR×CD3 bispecific antibodies were characterized in a cytokine release assay using PBMCs obtained from patients carrying CALRmut del52 and ins5.

PBMC isolation from blood: Peripheral blood mononuclear cells were isolated from whole blood (Sanguine Biosciences, Woburn, MA) using the provided protocol. Five donors were tested with the CALRmut del52 (Donors 1-5), and 1 donor was tested with CALRmut ins5 (Donor 6). Blood was delivered in sodium heparin (Na Hep) treated tubes and mixed. Tubes were spun at 1500×g for 10 minutes at room temperature prior to the removal of plasma. Blood was rinsed with ≈10 mL of PBS and then diluted 1:2 with more PBS. A total of 20 mL of the blood/PBS mixture was layered over 20 mL Ficoll and spun at 800×g for 20 minutes at room temperature with the brakes off. Plasma and PBS was removed, and PBMCs/Buffy Coat layer was collected. PBS was added up to 50 mL and tubes spun again at 400×g for 5 minutes at 4° C. Supernatant was removed, and 10 mL of red blood cell lysis buffer was added to each tube and allow to incubate for 5 minutes at 4° C. while rocking. PBS was added up to 50 mL and tubes spun at 250×g for 10 minutes at 4° C. Supernatant was removed, and pellets resuspended in 2 mL cold resuspension buffer (RPMI+40% FBS). Cells were counted and mixed with resuspension buffer to achieve a concentration of 10×106 cells/mL. Cells were frozen (30% DMSO in resuspension buffer diluted 1:2 in resuspension buffer) and stored in liquid nitrogen prior to its use.

For cytokine analysis, PBMCs were thawed in RPMI media (#11875-093; Gibco) supplemented with 10% FBS (#16140-071; Gibco), spun at 400×g for 5 minutes, and resuspended in RPMI/10% FBS media at a final concentration of 1.11 million cells/mL. Fc block (#422302; Biolegend) was added and cells were incubated at room temperature for 5 minutes (final concentration of cells≈1 million cells/mL). Cells were added into the 96-well assay plate (Corning #3879). Antibodies were prepared at 100 μg/mL (2× the Ab working concentration) in RPMI/10% FBS media. Serial dilutions were prepared with a 5× dilution for a 7-point dose curve for all antibodies. Controls included vibecotamab (CD123×CD3: Invitrogen: MA5-42248), mosunetuzumab, mock control IgG1 antibody (TT×CD3), negative control IgG1 anti-RSV antibody and untreated PBMCs. Prepared antibodies were added into the designated wells and the plate was incubated at 37° C. After 72 hours, plates were centrifuged and supernatant was removed for MSD analysis (Human Proinflammatory Panel kit #K151AEM-2). Supernatant was run on a custom MSD U-PLEX Immuno-Oncology Group 1 (human) Assay (K151AEM-2) to test the analytes GM-CSF, IL-1β, IL-4, IL-6, IL-8, IL-10, IL-12p70, IL-13, TNF-α. Plates were run according to manufacturer's recommendations and samples were diluted 1:2 in assay diluent. Plates were read on the MESO SECTOR S 600MM using their Methodical Mind™ with TeamLink™, ProductLink™, and Instrument Link™ connectivity Fall 2020 (v1.0.38) software. All data was analyzed using their MSD Discovery Workbench (LSR_4_0_13) software and GraphPad Prism (v10.2.3; Boston, MA) was used to plot the data.

Antibodies (vibecotamab, mosunetuzumab, mock control IgG1 antibody (TT×CD3), and negative control IgG1 anti-RSV antibody, and BsAb 18) were first tested for binding to CD4+ cells and CD8+ T cells T cells in PBMCs obtained from patients carrying CALRmut del52 and ins5. BsAb 18 showed binding activity to CD4+ cells and CD8+ T cells in both del52 and ins5 CALRmut patient PBMCs, similar to the binding of vibecotamab to these cells (data not shown). Binding of BsAb 18 was dose-dependent and the average EC50 values for CD4+ and CD8+ cell binding were found to be 2.31 and 1.73 μg/mL, respectively.

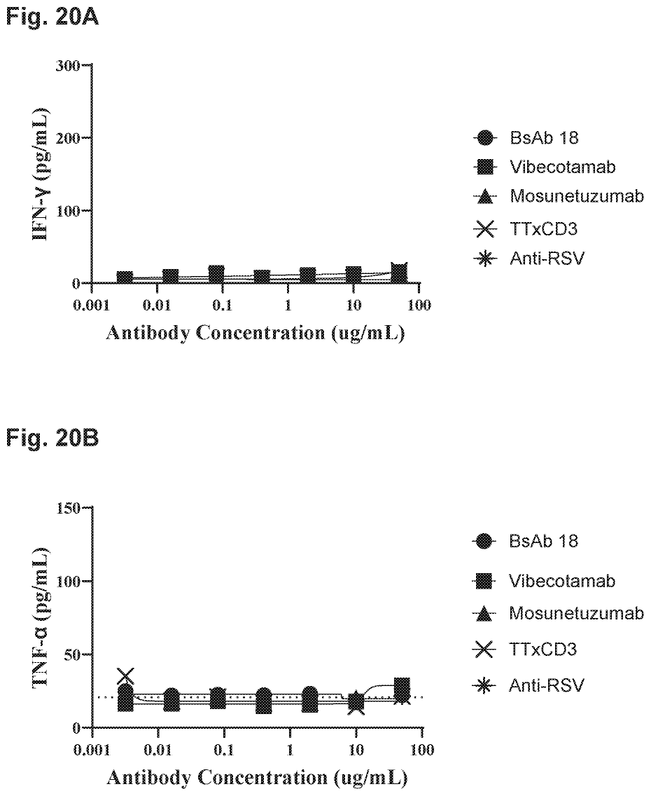

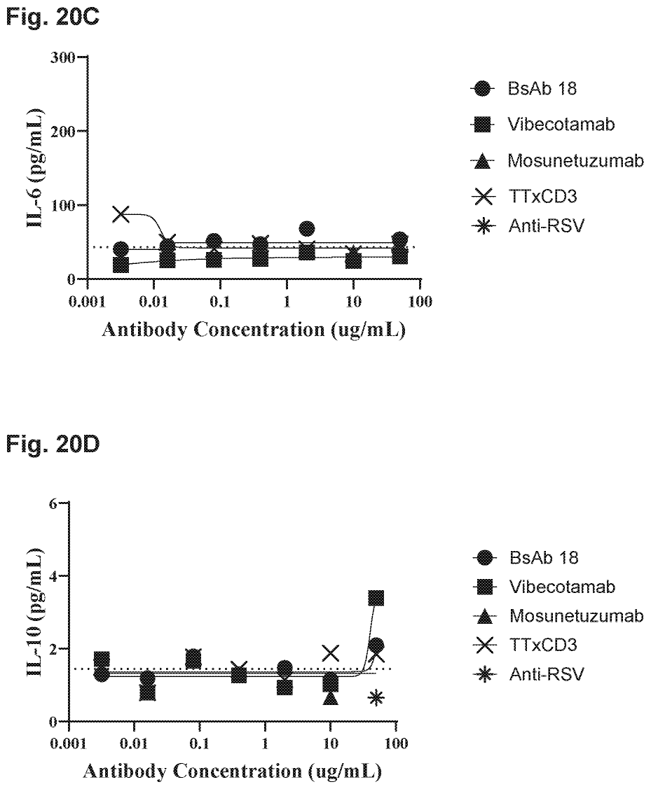

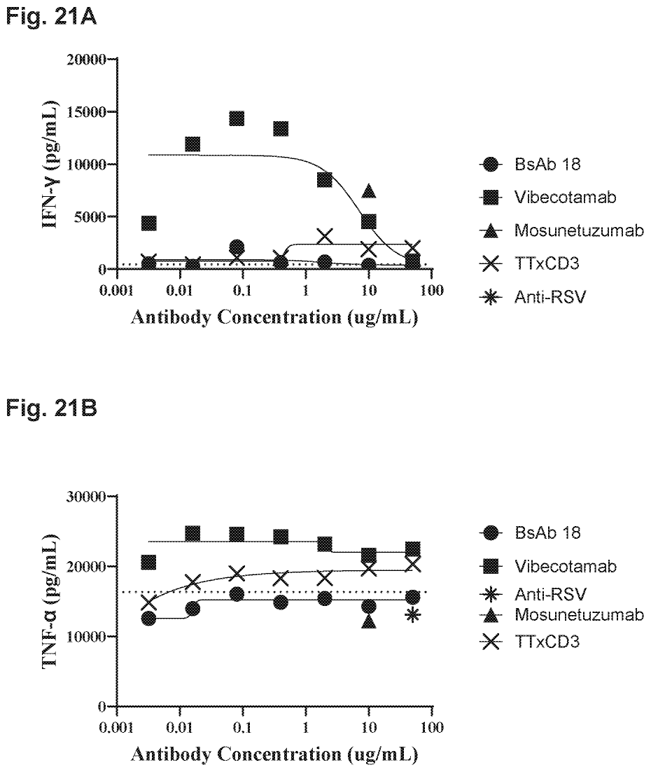

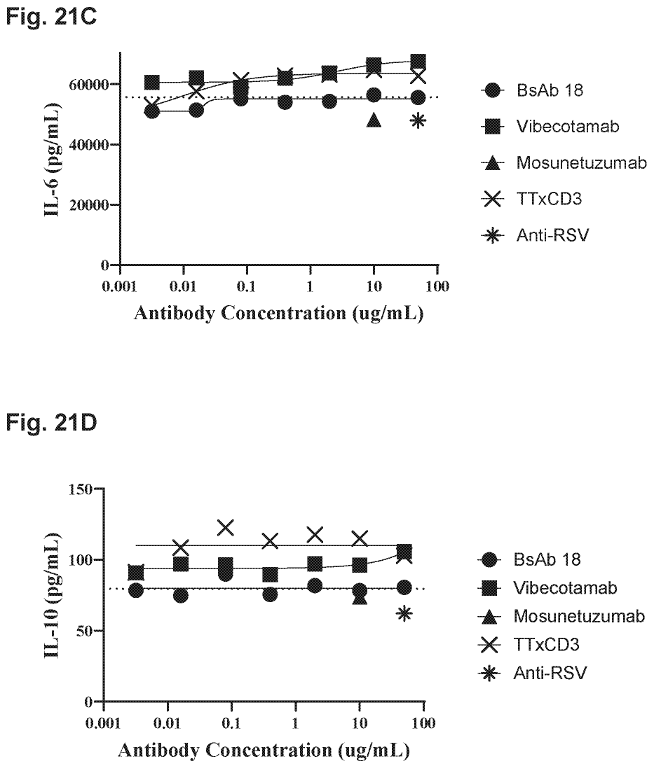

FIG. 20 shows cytokine analysis after incubation of antibodies with PBMCs isolated from a donor with CALRmut del52. FIG. 21 shows cytokine analysis after incubation of antibodies with PBMCs isolated from a donor with CALRmut ins5.

Results are shown for cytokines IFN-γ, TNF-α, IL-6, and IL-10 of one representative donor each. Further cytokines were tested, but data is not shown.

Despite binding to T cells, BsAb 18 did not cause high levels of CRS-associated cytokines in PBMCs from both CALRmut del52 and ins5 patients (FIG. 20 and FIG. 21). Together these results suggest that BsAb 18 effectively binds to patient-derived T cells yet does not induce high levels of CRS associated cytokines.

SEQUENCES
SEQ ID NO: 1 - Heavy chain variable region
EVQLVESGGGLVQPGGSLRLSCAASGFTFSSYAMSWVRQAPGKGLEWVSSVSGSGGNT
YYADSVKGRFTISRDNSKNTLYLQMSGLRAEDTAVYYCAKMEYNNKGYAYFFMEVW
GKGTTVTVSS
SEQ ID NO: 2 - Heavy chain CDR1 according to IMGT
GFTFSSYA
SEQ ID NO: 3 - Heavy chain CDR2 according to IMGT
VSGSGGNT
SEQ ID NO: 4 - Heavy chain CDR3 according to IMGT
AKMEYNNKGYAYFFMEV
SEQ ID NO: 5 - Heavy chain CDR1 according to Kabat
SYAMS
SEQ ID NO: 6 - Heavy chain CDR2 according to Kabat
SVSGSGGNTYYADSVKG
SEQ ID NO: 7 - Heavy chain CDR3 according to Kabat
MEYNNKGYAYFFMEV
SEQ ID NO: 8 - Heavy chain variable region
EVQLVESGGGLVQPGGSLRLSCAASGFTFSSYAMSWVRQAPGKGLEWVSSVSGSGGNT
YYADSVKGRFTISRDNSKNTLYLQMNSLRAEDTAVYYCAKMEYNNKGYAYFFMEVW
GKGTTVTVSS
SEQ ID NO: 9 - Heavy chain variable region
EVQLVESGGGLVQPGGSLRLSCAASGFTFSNYAMSWVRQAPGQGLEWASSISDSGGNT
YSADSMKGRFTISRDNSKNTLYLQMNSLRAEDTAVYYCARFIRSAKGYSYYYMDVWG
KGTMVTVSS
SEQ ID NO: 10 - Heavy chain CDR1 according to IMGT
GFTFSNYA
SEQ ID NO: 11 - Heavy chain CDR2 according to IMGT
ISDSGGNT
SEQ ID NO: 12 - Heavy chain CDR3 according to IMGT
ARFIRSAKGYSYYYMDV
SEQ ID NO: 13 - Heavy chain CDR1 according to Kabat
NYAMS
SEQ ID NO: 14 - Heavy chain CDR2 according to Kabat
SISDSGGNTYSADSMKG
SEQ ID NO: 15 - Heavy chain CDR3 according to Kabat
FIRSAKGYSYYYMDV
SEQ ID NO: 16 - Heavy chain variable region
EVQLVQSGAEVKKPGSSVKVSCKASGGTFRSFGISWVRQAPGQGLEWMGGFIPVLGTA
NYAQKFQGRVTIIADKSTNTAYMELSSLRSEDTAVYYCARRGNWNPFDPWGQGTLVTV
SS
SEQ ID NO: 17 - Heavy chain CDR1 according to IMGT
GGTFRSFG
SEQ ID NO: 18 - Heavy chain CDR2 according to IMGT
FIPVLGTA
SEQ ID NO: 19 - Heavy chain CDR3 according to IMGT
ARRGNWNPFDP
SEQ ID NO: 20 - Heavy chain CDR1 according to Kabat
SFGIS
SEQ ID NO: 21 - Heavy chain CDR2 according to Kabat
GFIPVLGTANYAQKFQG
SEQ ID NO: 22 - Heavy chain CDR3 according to Kabat
RGNWNPFDP
SEQ ID NO: 23 - Light chain variable region
DIQMTQSPSSLSASVGDRVTITCRASQSISSYLNWYQQKPGKAPKLLIYAASSLQSGVPSR
FSGSGSGTDFTLTISSLQPEDFATYYCQQSYSTPPTFGQGTKVEIK
SEQ ID NO: 24 - Light chain CDR1 according to IMGT
QSISSY
SEQ ID NO: 25 - Light chain CDR2 according to IMGT
AAS
SEQ ID NO: 26 - Light chain CDR3 according to IMGT
QQSYSTPPT
SEQ ID NO: 27 - Light chain CDR1 according to Kabat
RASQSISSYLN
SEQ ID NO: 28 - Light chain CDR2 according to Kabat
AASSLOS
SEQ ID NO: 29 - Light chain CDR3 according to Kabat
QQSYSTPPT
SEQ ID NO: 30 - CL region
RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 31 - V region VK1-39
DIQMTQSPSSLSASVGDRVTITCRASQSISSYLNWYQQKPGKAPKLLIYAASSLQSGVPSR
FSGSGSGTDFTLTISSLQPEDFATYYCQQSYSTP
SEQ ID NO: 32 - Light chain variable region
DIQMTQSPSSLSASVGDRVTITCRASQSISSYLNWYQQKPGKAPKLLIYAASSLQSGVPSR
FSGSGSGTDFTLTISSLQPEDFATYYCQQSYSTPPITFGQGTRLEIK
SEQ ID NO: 33 - Light chain CDR3 according to IMGT
QQSYSTPPIT
SEQ ID NO: 34 - V region VK3-15
EIVMTQSPATLSVSPGERATLSCRASQSVSSNLAWYQQKPGQAPRLLIYGASTRATGIPA
RFSGSGSGTEFTLTISSLQSEDFAVYYCQQYNNWP
SEQ ID NO: 35 - Light chain variable region
EIVMTQSPATLSVSPGERATLSCRASQSVSSNLAWYQQKPGQAPRLLIYGASTRATGIPA
RFSGSGSGTEFTLTISSLQSEDFAVYYCQQYNNWPWTFGQGTKVEIK
SEQ ID NO: 36 - Light chain CDR1 according to IMGT
QSVSSN
SEQ ID NO: 37 - Light chain CDR2 according to IMGT
GAS
SEQ ID NO: 38 - Light chain CDR3 according to IMGT
QQYNNWPWT
SEQ ID NO: 39 - V region VK3-20
EIVLTQSPGTLSLSPGERATLSCRASQSVSSSYLAWYQQKPGQAPRLLIYGASSRATGIPD
RFSGSGSGTDFTLTISRLEPEDFAVYYCQQYGSSP
SEQ ID NO: 40 - Light chain variable region
EIVLTQSPGTLSLSPGERATLSCRASQSVSSSYLAWYQQKPGQAPRLLIYGASSRATGIPD
RFSGSGSGTDFTLTISRLEPEDFAVYYCQQYGSSPWTFGQGTKVEIK
SEQ ID NO: 41 - Light chain CDR1 according to IMGT
QSVSSSY
SEQ ID NO: 42 - Light chain CDR2 according to IMGT
GAS
SEQ ID NO: 43 - Light chain CDR3 according to IMGT
QQYGSSPWT
SEQ ID NO: 44 - V region VL3-21
SYVLTQPPSVSVAPGETARITCGGDNIGRKSVYWYQQKSGQAPVLVIYYDSDRPSGIPER
FSGSNSGNTATLTISRVEAGDEADYYCQVWDGSSDH
SEQ ID NO: 45 - Light chain variable region
SYVLTQPPSVSVAPGETARITCGGDNIGRKSVYWYQQKSGQAPVLVIYYDSDRPSGIPER
FSGSNSGNTATLTISRVEAGDEADYYCQVWDGSSDHWVFGGGTKLTVL
SEQ ID NO: 46 - Light chain CDR1 according to IMGT
NIGRKS
SEQ ID NO: 47 - Light chain CDR2 according to IMGT
YDS
SEQ ID NO: 48 - Light chain CDR3 according to IMGT
QVWDGSSDHWV
SEQ ID NO: 49 - hinge region
EPKSCDKTHTCPPCP
SEQ ID NO: 50 - CHI region
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRV
SEQ ID NO: 51 - CH2 region
APELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAK
SEQ ID NO: 52 - CH2-DM region
APELGRGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAK
SEQ ID NO: 53 - CH3 region
GQPREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK
SEQ ID NO: 54 - CH3-DE region
GQPREPQVYTDPPSREEMTKNQVSLTCEVKGFYPSDIAVEWESNGQPENNYKTTPPVLD
SDGSFFLYSKLTVDKSRWQQGNVFSCSVMHEALHNHYTQKSLSLSPGK
SEQ ID NO: 55 - CH3-KK region
GQPREPQVYTKPPSREEMTKNQVSLKCLVKGFYPSDIAVEWESNGQPENNYKTTPPVLD
SDGSFFLYSKLTVDKSRWQQGNVFSCSVMHEALHNHYTQKSLSLSPGK
SEQ ID NO: 56 - CALR WT
MLLSVPLLLGLLGLAVAEPAVYFKEQFLDGDGWTSRWIESKHKSDFGKFVLSSGKFYG
DEEKDKGLQTSQDARFYALSASFEPFSNKGQTLVVQFTVKHEQNIDCGGGYVKLFPNSL
DQTDMHGDSEYNIMFGPDICGPGTKKVHVIFNYKGKNVLINKDIRCKDDEFTHLYTLIV
RPDNTYEVKIDNSQVESGSLEDDWDFLPPKKIKDPDASKPEDWDERAKIDDPTDSKPED
WDKPEHIPDPDAKKPEDWDEEMDGEWEPPVIQNPEYKGEWKPRQIDNPDYKGTWIHPE
IDNPEYSPDPSIYAYDNFGVLGLDLWQVKSGTIFDNFLITNDEAYAEEFGNETWGVTKA
AEKQMKDKQDEEQRLKEEEEDKKRKEEEEAEDKEDDEDKDEDEEDEEDKEEDEEEDVP
GQAKDEL
SEQ ID NO: 57 - CALR del52
MLLSVPLLLGLLGLAVAEPAVYFKEQFLDGDGWTSRWIESKHKSDFGKFVLSSGKFYG
DEEKDKGLQTSQDARFYALSASFEPFSNKGQTLVVQFTVKHEQNIDCGGGYVKLFPNSL
DQTDMHGDSEYNIMFGPDICGPGTKKVHVIFNYKGKNVLINKDIRCKDDEFTHLYTLIV
RPDNTYEVKIDNSQVESGSLEDDWDFLPPKKIKDPDASKPEDWDERAKIDDPTDSKPED
WDKPEHIPDPDAKKPEDWDEEMDGEWEPPVIQNPEYKGEWKPRQIDNPDYKGTWIHPE
IDNPEYSPDPSIYAYDNFGVLGLDLWQVKSGTIFDNFLITNDEAYAEEFGNETWGVTKA
AEKQMKDKQDEEQRTRRMMRTKMRMRRMRRTRRKMRRKMSPARPRTSCREACLQG
WTEA
SEQ ID NO: 58 - CALR ins5
MLLSVPLLLGLLGLAVAEPAVYFKEQFLDGDGWTSRWIESKHKSDFGKFVLSSGKFYG
DEEKDKGLQTSQDARFYALSASFEPFSNKGQTLVVQFTVKHEQNIDCGGGYVKLFPNSL
DQTDMHGDSEYNIMFGPDICGPGTKKVHVIFNYKGKNVLINKDIRCKDDEFTHLYTLIV
RPDNTYEVKIDNSQVESGSLEDDWDFLPPKKIKDPDASKPEDWDERAKIDDPTDSKPED
WDKPEHIPDPDAKKPEDWDEEMDGEWEPPVIQNPEYKGEWKPRQIDN
DPSIYAYDNFGVLGLDLWQVKSGTIFDNFLITNDEAYAEEFGNETWGVTKAAEKQMKD
KQDEEQRLKEEEEDKKRKEEEEAEDNCRRMMRTKMRMRRMRRTRRKMRRKMSPARP
RTSCREACLQGWTEA
SEQ ID NO: 59 - Heavy chain negative control RSV IgGI antibody
EVQLVESGGGVVQPGRSLRLSCAASGFTFSNYGMHWVRQAPGKGLEWVAVISYDGST
KYSADSLKGRFTISRDNSKNTLYLQMNSLRADDTAVYYCAKEGWSFDSSGYRSWEDS
WGQGTLVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKRVEPKSCDKTHT
CPPCPAPELGRGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPG
SEQ ID NO: 60 - Common light chain
DIQMTQSPSSLSASVGDRVTITCRASQSISSYLNWYQQKPGKAPKLLIYAASSLQSGVPSR
FSGSGSGTDFTLTISSLQPEDFATYYCQQSYSTPPTFGQGTKVEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
SEQ ID NO: 61 - Heavy chain negative control TT IgGI antibody
EVQLVETGAEVKKPGASVKVSCKASDYIFTKYDINWVRQAPGQGLEWMGWMSANTG
NTGYAQKFQGRVTMTRDTSINTAYMELSSLTSGDTAVYFCARSSLFKTETAPYYHFALD
VWGQGTTVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKRVEPKSCDKTH
TCPPCPAPELGRGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPG
SEQ ID NO: 62 - N-domain of CALR
MLLSVPLLLGLLGLAVAEPAVYFKEQFLDGDGWTSRWIESKHKSDFGKFVLSSGKFYG
DEEKDKGLQTSQDARFYALSASFEPFSNKGQTLVVQFTVKHEQNIDCGGGYVKLFPNSL
DQTDMHGDSEYNIMFGPDICGPGTKKVHVIFNYKGKNVLINKDIRCKDDEFTHLYTLIV
RPDNTYEVKIDNSQVESGSLE
SEQ ID NO: 63: CALR binding heavy chain
EVQLVESGGGLVQPGGSLRLSCAASGFTFSSYAMSWVRQAPGKGLEWVSSVSGSGGNT
YYADSVKGRFTISRDNSKNTLYLQMSGLRAEDTAVYYCAKMEYNNKGYAYFFMEVW
GKGTTVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRVEPKSCDKTHTC
PPCPAPELGRGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQP
REPQVYTDPPSREEMTKNQVSLTCEVKGFYPSDIAVEWESNGQPENNYKTTPPVLDSDG
SFFLYSKLTVDKSRWQQGNVFSCSVMHEALHNHYTQKSLSLSPG
SEQ ID NO: 64: CALR binding heavy chain
EVQLVESGGGLVQPGGSLRLSCAASGFTFSNYAMSWVRQAPGQGLEWASSISDSGGNT
YSADSMKGRFTISRDNSKNTLYLQMNSLRAEDTAVYYCARFIRSAKGYSYYYMDVWG
KGTMVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEPKSCDKTHTCP
PCPAPELGRGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPR
EPQVYTDPPSREEMTKNQVSLTCEVKGFYPSDIAVEWESNGQPENNYKTTPPVLDSDGS
FFLYSKLTVDKSRWQQGNVFSCSVMHEALHNHYTQKSLSLSPG
SEQ ID NO: 65 - CALR binding heavy chain
EVQLVESGGGLVQPGGSLRLSCAASGFTFSSYAMSWVRQAPGKGLEWVSSVSGSGGNT
YYADSVKGRFTISRDNSKNTLYLQMNSLRAEDTAVYYCAKMEYNNKGYAYFFMEVW
GKGTTVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRVEPKSCDKTHTC
PPCPAPELGRGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAK
GQPREPQVYTDPPSREEMTKNQVSLTCEVKGFYPSDIAVEWESNGQPENNYKTTPPVLD
SDGSFFLYSKLTVDKSRWQQGNVFSCSVMHEALHNHYTQKSLSLSPG
SEQ ID NO: 66 - CD3 binding heavy chain
EVQLVQSGAEVKKPGSSVKVSCKASGGTFRSFGISWVRQAPGQGLEWMGGFIPVLGTA
NYAQKFQGRVTIIADKSTNTAYMELSSLRSEDTAVYYCARRGNWNPFDPWGQGTLVTV
SSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPKSCDKTHTCPPCPAPELG
RGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTKP
PSREEMTKNQVSLKCLVKGFYPSDIAVEWESNGQPENNYKTTPPVLDSDGSFFLYSKLT
VDKSRWQQGNVFSCSVMHEALHNHYTQKSLSLSPG

1. A polypeptide comprising the amino acid sequence as set forth in SEQ ID NO: 1, 8, or 9, or a variant thereof.

2. A method for producing a variant of a polypeptide of claim 1, wherein the method comprises:
making a modification in the amino acid sequence as set forth in SEQ ID NO: 1, 8, or 9.

3. (canceled)

4. A CALR binding domain comprising a polypeptide according to claim 1.

5. The CALR binding domain according to claim 4, wherein the CALR binding domain further comprises a polypeptide comprising the amino acid sequence as set forth in SEQ ID NO: 23, or having at least 80%, at least 85%, at least 90%, or at least 95% sequence identity thereto.

6. A CALR binding domain comprising a heavy chain variable region comprising the heavy chain CDR1 (HCDR1), heavy chain CDR2 (HCDR2), and heavy chain CDR3 (HCDR3) of a heavy chain variable region having the amino acid sequence selected from SEQ ID NOS: 1, 8, and 9, wherein each of the HCDRs may comprise at most three, two, or one amino acid variations.

7. The CALR binding domain according to claim 6, wherein the CALR binding domain comprises a heavy chain variable region comprising:
a) heavy chain CDR1 (HCDR1), heavy chain CDR2 (HCDR2), and heavy chain CDR3 (HCDR3), having the amino acid sequence as set forth in SEQ ID NO: 2, SEQ ID NO: 3, and SEQ ID NO: 4, respectively, or heavy chain CDR1 (HCDR1), heavy chain CDR2 (HCDR2), and heavy chain CDR3 (HCDR3), having the amino acid sequence as set forth in SEQ ID NO: 5, SEQ ID NO: 6, and SEQ ID NO: 7, respectively; or
b) heavy chain CDR1 (HCDR1), heavy chain CDR2 (HCDR2), and heavy chain CDR3 (HCDR3), having the amino acid sequence as set forth in SEQ ID NO: 10, SEQ ID NO: 11, and SEQ ID NO: 12, respectively, or heavy chain CDR1 (HCDR1), heavy chain CDR2 (HCDR2), and heavy chain CDR3 (HCDR3), having the amino acid sequence as set forth in SEQ ID NO: 13, SEQ ID NO: 14, and SEQ ID NO: 15, respectively,
wherein each of the HCDRs may comprise at most three, two, or one amino acid variations.

8. The CALR binding domain according to claim 6, wherein the CALR binding domain comprises a heavy chain variable region having the amino acid sequence as set forth in any one of SEQ ID NO: 1, 8, or 9, or having at least 80%, at least 85%, at least 90%, or at least 95% sequence identity thereto.

9. The CALR binding domain according to claim 6, wherein the CALR binding domain comprises a light chain variable region comprising the light chain CDR1 (LCDR1), light chain CDR2 (LCDR2), and light chain CDR3 (LCDR3) of a light chain variable region having the amino acid sequence as set forth in SEQ ID NO: 23, wherein each of the LCDRs may comprise at most three, two, or one amino acid variations.

10. The CALR binding domain according to claim 6, wherein the CALR binding domain comprises a light chain variable region comprising light chain CDR1 (LCDR1), light chain CDR2 (LCDR2), and light chain CDR3 (LCDR3), having an amino acid sequence as set forth in SEQ ID NO: 24, SEQ ID NO: 25, and SEQ ID NO: 26, respectively, or light chain CDR1 (LCDR1), light chain CDR2 (LCDR2), and light chain CDR3 (LCDR3), having an amino acid sequence as set forth in SEQ ID NO: 27, SEQ ID NO: 28, and SEQ ID NO: 29, respectively, wherein each of the LCDRs may comprise at most three, two, or one amino acid variations.

11. (canceled)

12. A method for producing a variant of a CALR binding domain of claim 6, wherein the method comprises:
making a modification in the amino acid sequence as set forth in any one of SEQ ID NOS: 1, 8, or 9, and/or in the amino acid sequence as set forth in SEQ ID NO: 23;
testing the modified CALR binding domain for binding to human wildtype and/or mutant CALR; and
selecting the modified CALR binding domain if it binds to human wildtype and/or mutant CALR.

13. (canceled)

14. A binding moiety comprising a CALR binding domain to claim 4, in particular wherein the binding moiety is a monospecific binding moiety, or a bispecific binding moiety, in particular a bivalent monospecific antibody or a bivalent bispecific antibody.

15-16. (canceled)

17. A bispecific binding moiety comprising a CALR binding domain and a CD3 binding domain, wherein the bispecific binding moiety has one or more properties selected from:
high T cell-mediated tumor cell killing;
no or low internalization by human T cells;
low potency to induce cytokine release syndrome;
low clearance in a cynomolgus monkey pharmacokinetics (PK) study.

18. The bispecific binding moiety according to claim 17, wherein the CALR binding domain binds to wildtype CALR, and to mutant CALR.

19. The bispecific binding moiety according to claim 18, wherein the amino acid sequence of the wildtype CALR is set forth in SEQ ID NO: 56, wherein the mutant CALR is human CALRmut del52, the amino acid sequence of which is set forth in SEQ ID NO: 57, and/or human CALRmut ins5, the amino acid sequence of which is set forth in SEQ ID NO: 58.

20-30. (canceled)

31. A bispecific binding moiety comprising a CALR binding domain and a CD3 binding domain, wherein the CALR binding domain comprises a heavy chain variable region comprising the heavy chain CDR1 (HCDR1), heavy chain CDR2 (HCDR2), and heavy chain CDR3 (HCDR3) of a heavy chain variable region having the amino acid sequence as set forth in SEQ ID NOS: 1, 8, or 9, wherein each of the HCDRs may comprise at most three, two, or one amino acid variations.

32. The bispecific binding moiety according to claim 31, wherein the CALR binding domain comprises a heavy chain variable region comprising:
a) heavy chain CDR1 (HCDR1), heavy chain CDR2 (HCDR2), and heavy chain CDR3 (HCDR3), having the amino acid sequence as set forth in SEQ ID NO: 2, SEQ ID NO: 3, and SEQ ID NO: 4, respectively, or heavy chain CDR1 (HCDR1), heavy chain CDR2 (HCDR2), and heavy chain CDR3 (HCDR3), having the amino acid sequence as set forth in SEQ ID NO: 5, SEQ ID NO: 6, and SEQ ID NO: 7, respectively; or
b) heavy chain CDR1 (HCDR1), heavy chain CDR2 (HCDR2), and heavy chain CDR3 (HCDR3), having the amino acid sequence as set forth in SEQ ID NO: 10, SEQ ID NO: 11, and SEQ ID NO: 12, respectively, or heavy chain CDR1 (HCDR1), heavy chain CDR2 (HCDR2), and heavy chain CDR3 (HCDR3), having the amino acid sequence as set forth in SEQ ID NO: 13, SEQ ID NO: 14, and SEQ ID NO: 15, respectively,
wherein each of the HCDRs may comprise at most three, two, or one amino acid variations.

33. The bispecific binding moiety according claim 31, wherein the CALR binding domain comprises a heavy chain variable region having the amino acid sequence as set forth in SEQ ID NO: 1, 8, or 9, or having at least 80%, or at least 85%, or at least 90%, or at least 95% sequence identity thereto.

34. The bispecific binding moiety according to claim 31, wherein the CALR binding domain comprises a light chain variable region comprising the light chain CDR1 (LCDR1), light chain CDR2 (LCDR2), and light chain CDR3 (LCDR3) of a light chain variable region having the amino acid sequence as set forth in SEQ ID NO: 23, wherein each of the LCDRs may comprise at most three, two, or one amino acid variations.

35. The bispecific binding moiety according to claim 31, wherein the CALR binding domain comprises a light chain variable region comprising light chain CDR1 (LCDR1), light chain CDR2 (LCDR2), and light chain CDR3 (LCDR3), having an amino acid sequence as set forth in SEQ ID NO: 24, SEQ ID NO: 25, and SEQ ID NO: 26, respectively, or light chain CDR1 (LCDR1), light chain CDR2 (LCDR2), and light chain CDR3 (LCDR3), having an amino acid sequence as set forth in SEQ ID NO: 27, SEQ ID NO: 28, and SEQ ID NO: 29, respectively wherein each of the LCDRs may comprise at most three, two, or one amino acid variations.

36. The bispecific binding moiety according to claim 31, wherein the CALR binding domain comprises a light chain variable region having the amino acid sequence as set forth in SEQ ID NO: 23, or having at least 80%, or at least 85%, or at least 90%, or at least 95% sequence identity thereto.

37. The bispecific binding moiety according to claim 31, wherein the CD3 binding domain comprises a heavy chain variable region comprising the heavy chain CDR1 (HCDR1), heavy chain CDR2 (HCDR2), and heavy chain CDR3 (HCDR3) of a heavy chain variable region having the amino acid sequence as set forth in SEQ ID NO: 16, wherein each of the HCDRs may comprise at most three, two, or one amino acid variations.

38. The bispecific binding moiety according to claim 31, wherein the CD3 binding domain comprises a heavy chain variable region comprising heavy chain CDR1 (HCDR1), heavy chain CDR2 (HCDR2), and heavy chain CDR3 (HCDR3), having the amino acid sequence as set forth in SEQ ID NO: 17, SEQ ID NO: 18, and SEQ ID NO: 19, respectively, or heavy chain CDR1 (HCDR1), heavy chain CDR2 (HCDR2), and heavy chain CDR3 (HCDR3), having the amino acid sequence as set forth in SEQ ID NO: 20, SEQ ID NO: 21, and SEQ ID NO: 22, respectively,
wherein each of the HCDRs may comprise at most three, two, or one amino acid variations.

39. The bispecific binding moiety according to claim 31, wherein the CD3 binding domain comprises a heavy chain variable region having the amino acid sequence as set forth in SEQ ID NO: 16, or having at least 80%, or at least 85%, or at least 90%, or at least 95% sequence identity thereto.

40. The bispecific binding moiety according to claim 31, wherein the CD3 binding domain comprises a light chain variable region comprising the light chain CDR1 (LCDR1), light chain CDR2 (LCDR2), and light chain CDR3 (LCDR3) of a light chain variable region having the amino acid sequence as set forth in SEQ ID NO: 23, wherein each of the LCDRs may comprise at most three, two, or one amino acid variations.

41. The bispecific binding moiety according to claim 31, wherein the CD3 binding domain comprises a light chain variable region comprising light chain CDR1 (LCDR1), light chain CDR2 (LCDR2), and light chain CDR3 (LCDR3), having the amino acid sequence as set forth in SEQ ID NO: 24, SEQ ID NO: 25, and SEQ ID NO: 26, respectively, or light chain CDR1 (LCDR1), light chain CDR2 (LCDR2), and light chain CDR3 (LCDR3), having the amino acid sequence as set forth in SEQ ID NO: 27, SEQ ID NO: 28, and SEQ ID NO: 29, respectively, wherein each of the LCDRs may comprise at most three, two, or one amino acid variations.

42. The bispecific binding moiety according to claim 31, wherein the CD3 binding domain comprises a light chain variable region having the amino acid sequence as set forth in SEQ ID NO: 23, or having at least 80%, or at least 85%, or at least 90%, or at least 95% sequence identity thereto.

43. A bispecific binding moiety comprising a CALR binding domain and a CD3 binding domain, wherein the CALR binding domain comprises a heavy chain variable region having the amino acid sequence selected from SEQ ID NO: 1, 8, or 9, the CD3 binding domain comprises a heavy chain variable region having the amino acid sequence as set forth in SEQ ID NO: 16, and both the CALR binding domain and the CD3 binding domain comprise a light chain variable region having the amino acid sequence as set forth in SEQ ID NO: 23.

44-45. (canceled)

46. A bispecific binding moiety that binds CALR and CD3, wherein the bispecific binding moiety competes with a bispecific binding moiety of any one of claims 17, 31 or 43 for binding to CALR and/or CD3.

47. A pharmaceutical composition comprising an effective amount of the bispecific binding moiety according to any one of claims 17, 31 or 43, and a pharmaceutically acceptable carrier.

48-49. (canceled)

50. A method for treating a disease, comprising administering an effective amount of the pharmaceutical composition according to claim 47, to a subject in need thereof.

51. A method for treating cancer, comprising administering an effective amount of the pharmaceutical composition according to claim 47, to a subject in need thereof.

52. A nucleic acid sequence encoding a heavy chain variable region as defined in claim 31.

53. A vector comprising a nucleic acid sequence as claimed in claim 52.

54. The vector according to claim 53, wherein the vector further comprises a nucleic acid sequence encoding a CH1 region, in particular encoding a CH1 region, hinge, CH2 region, and CH3 region, and preferably wherein the vector further comprises at least one nucleic acid sequence encoding a light chain variable region, in particular encoding a light chain variable region and a CL region.

55-56. (canceled)

57. A cell comprising a nucleic acid sequence encoding the heavy chain variable region of a CALR binding domain as defined in claim 31 and a nucleic acid sequence encoding the heavy chain variable region of a CD3 binding domain wherein the CD3 binding domain comprises a heavy chain variable region comprising the heavy chain CDR1 (HCDR1), heavy chain CDR2 (HCDR2), and heavy chain CDR3 (HCDR3) of a heavy chain variable region having the amino acid sequence as set forth in SEQ ID NO: 16, wherein each of the HCDRs may comprise at most three, two, or one amino acid variations.

58. The cell according to claim 57, wherein the cell further comprises a nucleic acid sequence encoding a CH1 region, in particular a nucleic acid sequence encoding a CH1 region, hinge, CH2 region, and CH3 region, and preferably wherein the cell further comprises at least one nucleic acid sequence encoding a light chain variable region, wherein the light chain variable region (a) comprises a light chain CDR1 (LCDR), light chain CDR2 (LCDR2), and light chain CDR3 (LCDR3) of a light chain variable region having the amino acid sequence as set forth in SEQ ID NO: 23, wherein each of the LCDRs may comprise at most three, two, or one amino acid variations; or (b) comprises a light chain CDR1 (LCDR1), light chain CDR2 (LCDR2), and light chain CDR3 (LCDR3), having an amino acid sequence as set forth in SEQ ID NO: 24, SEQ ID NO: 25, and SEQ ID NO: 26, respectively, or light chain CDR1 (LCDR1), light chain CDR2 (LCDR2), and light chain CDR3 (LCDR3), having an amino acid sequence as set forth in SEQ ID NO: 27, SEQ ID NO: 28, and SEQ ID NO: 29, respectively wherein each of the LCDRs may comprise at most three, two, or one amino acid variations, or comprises (a) and a CL region or (b) and a CL region.

59. (canceled)

60. A cell producing a bispecific binding moiety as claimed in any one of claims 17, 31 or 43, in particular wherein the cell is a recombinant cell comprising the vector as claimed in claim 53.

61. (canceled)
